(19) United States
(12) Patent Application Publication
(10) Pub. No.: US 2023/0381162 A1
(43) Pub. Date: Nov. 30, 2023

(54) TREATMENTS OF ANGIOEDEMA

(71) Applicant: KALVISTA PHARMACEUTICALS LIMITED, Wiltshire (GB)

(72) Inventors: John Alexander COOPER, Wiltshire (GB); Edward Paul FEENER, North Reading, MA (US); Andreas MAETZEL, Chapel Hill, NC (US); Sally Louise MARSH, Wiltshire (GB); Michael David SMITH, Salt Lake City, UT (US); Christopher Martyn YEA, Wiltshire (GB)

(21) Appl. No.: 18/249,076

(22) PCT Filed: Oct. 22, 2021
(86) PCT No.: PCT/GB2021/052746

§ 371 Date: Apr. 14, 2023

Related U.S. Application Data
(60) Provisional application No. 63/104,919, filed on Oct. 23, 2020.
(60) Provisional application No. 63/147,595, filed on Feb. 9, 2021.
(60) Provisional application No. 63/212,224, filed on Jun. 18, 2021.
(60) Provisional application No. 63/220,747, filed on Jul. 12, 2021.

(51) Int. Cl. A61K 31/4439; A61K 9/20; A61P 7/00

(52) U.S. Cl. CPC A61K 31/4439; A61K 9/2018; A61P 7/00

(57) ABSTRACT

The present invention relates to treatments of bradykinin-mediated angioedema. In particular, the present invention provides on-demand treatments of bradykinin-mediated angioedema by orally administering a plasma kallikrein inhibitor to a patient in need thereof on-demand as an oral dosage form particularly suitable for patients who may struggle to swallow tablets.

The present invention relates to treatments of bradykinin-mediated angioedema. In particular, the present invention provides on-demand treatments of bradykinin-mediated angioedema by orally administering a plasma kallikrein inhibitor to a patient in need thereof on-demand as an oral dosage form particularly suitable for patients who may struggle to swallow tablets.

### BACKGROUND TO THE INVENTION

Inhibitors of plasma kallikrein have a number of therapeutic applications, particularly in the treatment of bradykinin-mediated angioedema such as hereditary angioedema and bradykinin-mediated angioedema non-hereditary (BK-AEnH).

Plasma kallikrein is a trypsin-like serine protease that can liberate kinins from kininogens (see K. D. Bhoola et al., “Kallikrein-Kinin Cascade”, Encyclopedia of Respiratory Medicine, p483-493; J. W. Bryant et al., “Human plasma kallikrein-kinin system: physiological and biochemical parameters” Cardiovascular and haematological agents in medicinal chemistry, 7, p234-250, 2009; K. D. Bhoola et al., Pharmacological Rev., 1992, 44, 1; and D. J. Campbell, “Towards understanding the kallikrein-kinin system: insights from the measurement of kinin peptides”, Brazilian Journal of Medical and Biological Research 2000, 33, 665-677). It is an essential member of the intrinsic blood coagulation cascade, although its role in this cascade does not involve the release of bradykinin or enzymatic cleavage. Plasma prekallikrein is encoded by a single gene and can be synthesized in the liver, as well as other tissues. It is secreted by hepatocytes as an inactive plasma prekallikrein that circulates in plasma as a heterodimer complex bound to high molecular weight kininogen (HK) which is activated to give the active plasma kallikrein. This contact activation system (or contact system) can be activated by negatively charged surfaces that activate Factor XII (FXII) to Factor XIIa (FXIIa), by certain proteases e.g. plasmin (Hofman et al., Clin Rev Allergy Immunol 2016), which may not require negative surfaces, or by misfolded proteins (Maas et al., J Clinical Invest 2008). FXIIa mediates conversion of plasma prekallikrein to plasma kallikrein and the subsequent cleavage of high molecular weight kininogen (HK) to generate bradykinin, a potent inflammatory hormone. Kinins are potent mediators of inflammation that act through G protein-coupled receptors and antagonists of kinins (such as bradykinin receptor antagonists) have previously been investigated as potential therapeutic agents for the treatment of a number of disorders (F. Marceau and D. Regoli, Nature Rev., Drug Discovery, 2004, 3, 845-852).

Plasma kallikrein is thought to play a role in a number of inflammatory disorders. For example, the plasma kallikrein-kinin system is abnormally abundant in patients with advanced diabetic macular edema. It has been reported that plasma kallikrein contributes to retinal vascular dysfunctions in diabetic rats (A. Clermont et al., “Plasma kallikrein mediates retinal vascular dysfunction and induces retinal thickening in diabetic rats” Diabetes, 2011, 60, 1590-98). Furthermore, administration of the plasma kallikrein inhibitor ASP-440 ameliorated both retinal vascular permeability and retinal blood flow abnormalities in diabetic rats. Therefore a plasma kallikrein inhibitor should have utility as a treatment to reduce retinal vascular permeability associated with diabetic retinopathy and diabetic macular edema.

Other complications of diabetes such as cerebral haemorrhage, nephropathy, cardiomyopathy and neuropathy, all of which have associations with plasma kallikrein may also be considered as targets for a plasma kallikrein inhibitor.

The major inhibitor of plasma kallikrein is the serpin C1 esterase inhibitor. Patients who present with a genetic deficiency in C1 esterase inhibitor suffer from hereditary angioedema (HAE) which results in intermittent swelling of face, hands, throat, gastro-intestinal tract and genitals. Blisters formed during acute episodes contain high levels of plasma kallikrein which cleaves high molecular weight kininogen (HK) liberating bradykinin leading to increased vascular permeability. “Hereditary angioedema” can thus be defined as any disorder characterised by recurrent episodes of bradykinin-mediated angioedema (e.g. severe swelling) caused by an inherited dysfunction/fault/mutation. There are currently three known categories of HAE: (i) HAE type 1, (ii) HAE type 2, and (iii) normal C1 inhibitor HAE (normal C1-Inh HAE). However, the HAE field is developing quickly and further types of HAE might be defined in the future.

Without wishing to be bound by theory, it is thought that HAE type 1 is caused by mutations in the SERPING1 gene that lead to reduced levels of C1 inhibitor in the blood. Without wishing to be bound by theory, it is thought that HAE type 2 is caused by mutations in the SERPING1 gene that lead to dysfunction of the C1 inhibitor in the blood. Without wishing to be bound by theory, the cause of normal C1-Inh HAE is less well defined and the underlying genetic dysfunction/fault/mutation can sometimes remain unknown. What is known is that the cause of normal C1-Inh HAE is not related to reduced levels or dysfunction of the C1 inhibitor (in contrast to HAE types 1 and 2). Normal C1-Inh HAE can be diagnosed by reviewing the family history and noting that angioedema has been inherited from a previous generation (and thus it is hereditary angioedema). Normal C1-Inh HAE can also be diagnosed by determining that there is a dysfunction/fault/mutation in a gene other than those related to C1 inhibitor. For example, it has been reported that dysfunction/fault/mutation with plasminogen can cause normal C1-Inh HAE (see e.g. Veronez et al., Front Med (Lausanne). 2019 Feb. 21; 6:28. doi: 10.3389/fmed.2019.00028; or Recke et al., Clin Transl Allergy. 2019 Feb. 14; 9:9. doi: 10.1186/s13601-019-0247-x.). It has also been reported that dysfunction/fault/mutation with Factor XII can cause normal C1-Inh HAE (see e.g. Mansi et al., The Association for the Publication of the Journal of Internal Medicine Journal of Internal Medicine, 2015, 277; 585-593; or Maat et al. J Thromb Hoemost. 2019 Jan.;17(1):183-194. doi: 10.1111/jth.14325).

Acute HAE attacks normally progress through three key clinically distinct stages: an initial prodromal stage (that can typically last for up to 12 hours), followed by a swelling stage, and then an absorption stage. A majority of HAE attacks announce themselves with prodromal symptoms. Two thirds of prodromes appeared less than 6 hours before a HAE attack and no prodromes occur more than 24 hours before a HAE attack (Magerl et al., Clinical and Experimental Dermatology 2014, 39, 298-303). For example, the following prodromal symptoms may start to be observed: a slight swelling (particularly affecting the face and neck), a typical type of abdominal pain, a typical reddening of the skin called “erythema marginatum”. An attack is fully developed when it has reached maximum swelling and maximum expression of pain (e.g. abdominal attack), discomfort (e.g. peripheral attack) or threat to life (e.g. laryngeal attack). Once the attack has reached its peak, the subsequent time period to normalization is determined by the time it takes for the swelling to disappear and the liquid that has penetrated the tissues to be reabsorbed.

However, angioedemas are not necessarily inherited. Indeed, another class of angioedema is bradykinin-mediated angioedema non-hereditary (BK-AEnH), which is not caused by an inherited genetic dysfunction/fault/mutation. Often the underlying cause of BK-AEnH is unknown and/or undefined. However, the signs and symptoms of BK-AEnH are similar to those of HAE, which without being bound by theory, is thought to be on account of the shared bradykinin-mediated pathway between HAE and BK-AEnH. Specifically, BK-AEnH is characterised by recurrent acute attacks where fluids accumulate outside of the blood vessels, blocking the normal flow of blood or lymphatic fluid and causing rapid swelling of tissues such as in the hands, feet, limbs, face, intestinal tract, airway or genitals.

Specific types of BK-AEnH include: non-hereditary angioedema with normal C1 Inhibitor (AE-nC1 Inh), which can be environmental, hormonal, or drug-induced; acquired angioedema; anaphylaxis associated angioedema; angiotensin converting enzyme (ACE) inhibitor-induced angioedema; dipeptidyl peptidase-4 inhibitor-induced angioedema; and tPA-induced angioedema (tissue plasminogen activator-induced angioedema). However, reasons why these factors and conditions cause angioedema in only a relatively small proportion of individuals are unknown.

Environmental factors that can induce AE-nC1 Inh include air pollution (Kedarisetty et al., Otolaryngol Head Neck Surg. 2019 April 30:194599819846446. doi: 10.1177/0194599819846446) and silver nanoparticles such as those used as antibacterial components in healthcare, biomedical and consumer products (Long et al., Nanotoxicology. 2016; 10(4):501-11. doi: 10.3109/17435390.2015.1088589).

Various publications suggest a link between the bradykinin and contact system pathways and BK-AEnHs, and also the potential efficacy of treatments, see e.g.: Bas et al. (N Engl J Med 2015); Leibfried and Kovary. (J Pharm Pract 2017); van den Elzen et al. (Clinic Rev Allerg Immunol 2018); Han et al. (JCI 2002).

tPA-induced angioedema is discussed in various publications as being a potentially life-threatening complication following thrombolytic therapy in acute stroke victims (see e.g. Simão et al., Blood. 2017 Apr. 20; 1 29(16):2280-2290. doi: 10.1182/blood-2016-09-740670; Fröhlich et al., Stroke. 2019 Jun. 11:STROKEAHA119025260. doi: 10.1161/STROKEAHA.119.025260; Rathbun, Oxf Med Case Reports. 2019 Jan. 24; 2019(1):omy112. doi: 10.1093/omcr/omy112; Lekoubou et al., Neurol Res. 2014 Jul.;36(7):687-94. doi: 10.1179/1743132813Y.0000000302; Hill et al., Neurology. 2003 May 13;60(9):1525-7).

Stone et al. (Immunol Allergy Clin North Am. 2017 August; 37(3):483-495.) reports that certain drugs can cause angioedema.

Scott et al. (Curr Diabetes Rev. 2018; 14(4):327-333. doi: 10.2174/1573399813666170214113856) reports cases of dipeptidyl Peptidase-4 Inhibitor induced angioedema.

Hermanrud et al. (BMJ Case Rep. 2017 Jan. 10; 2017. pii: bcr2016217802) reports recurrent angioedema associated with pharmacological inhibition of dipeptidyl peptidase IV and also discusses acquired angioedema related to angiotensin-converting enzyme inhibitors (ACEI-AAE). Kim et al. (Basic Clin Pharmacol Toxicol. 2019 Jan.;124(1):115-122. doi: 10.1111/bcpt.13097) reports angiotensin II receptor blocker (ARB)-related angioedema. Reichman etal., (Pharmacoepidemiol Drug Saf. 2017 Oct.;26(10):1190-1196. doi: 10.1002/pds.4260) also reports angioedema risk for patients taking ACE inhibitors, ARB inhibitors and beta blockers. Diestro et al. (J Stroke Cerebrovasc Dis. 2019 May;28(5):e44-e45. doi: 10.1016/j.jstrokecerebrovasdis.2019.01.030) also reports a possible association between certain angioedemas and ARBs.

Giard et al. (Dermatology. 2012; 225(1):62-9. doi: 10.1159/000340029) reports that bradykinin-mediated angioedema can be precipitated by oestrogen contraception.

Cinryze® and Haegarda® contain a C1 esterase inhibitor and are authorised to prevent acute HAE attacks (i.e. prophylactic treatment). Treatment with Cinryze® requires the preparation of a solution from a powder, which is then injected every 3 or 4 days. Similarly, treatment with Haegarda® requires the preparation of a solution from a powder, which is then injected twice a week. It is not always possible for a patient to self-administer these treatments, and if this is the case, the patient is required to visit a clinic for treatment. Thus, both of these prophylactic treatments suffer from high patient burden. Additionally, the FDA packet insert for Haegarda® states that it “should not be used to treat an acute HAE attack”, and therefore a patient may require additional therapy if a HAE attack develops.

Berinert® and Ruconest® contain a C1 esterase inhibitor and are authorised to treat acute HAE attacks. Both of these treatments also involve the preparation of an injectable solution followed by injection. This process can be burdensome on the patient, especially when the patient is suffering from an acute HAE attack. Self-administration of the dosage amount is also not always possible, and if it is not, administration of the drug can be substantially delayed thus increasing the severity of the acute HAE attack for the patient.

There is a lack of approved treatments for plasma kallikrein-related disorders such as HAE and BK-AEnH. In particular, there is a lack of any approved oral treatment for plasma kallikrein-related disorders (such as HAE, BK-AEnH). Berotralstat (BCX7353) is a plasma kallikrein inhibitor being investigated as a once-daily oral treatment for the prevention of HAE attacks. Hwang et al. (Immunotherapy (2019) 11(17), 1439-1444) states that higher doses of berotralstat were associated with more gastrointestinal adverse effects indicating increased toxicity at higher levels.

Synthetic and small molecule plasma kallikrein inhibitors have been described previously, for example by Garrett et al. (“Peptide aldehyde. . . .” J. Peptide Res. 52, 62-71 (1998)), T. Griesbacher et al. (“Involvement of tissue kallikrein but not plasma kallikrein in the development of symptoms mediated by endogenous kinins in acute pancreatitis in rats” British Journal of Pharmacology 137, 692-700 (2002)), Evans (“Selective dipeptide inhibitors of kallikrein” WO03/076458), Szelke et al. (“Kininogenase inhibitors” WO92/04371), D. M. Evans et al. (Immunolpharmacology, 32, p115-116 (1996)), Szelke et al. (“Kininogen inhibitors” WO95/07921), Antonsson et al. (“New peptides derivatives” WO94/29335), J. Corte et al. (“Six membered heterocycles useful as serine protease inhibitors” WO2005/123680), J. Sturzbecher etal. (Brazilian J. Med. Biol. Res 27, p1929-34 (1994)), Kettner et al. (U.S. Pat. No. 5,187,157), N. Teno et al. (Chem. Pharm. Bull. 41, p1079-1090(1993)), W. B. Young etal. (“Small molecule inhibitors of plasma kallikrein” Bioorg. Med. Chem. Letts. 16, p2034-2036 (2006)), Okada etal. (“Development of potent and selective plasmin and plasma kallikrein inhibitors and studies on the structure-activity relationship” Chem. Pharm. Bull. 48, p1964-72 (2000)), Steinmetzer et al. (“Trypsin-like serine protease inhibitors and their preparation and use” WO08/049595), Zhang et al. (“Discovery of highly potent small molecule kallikrein inhibitors” Medicinal Chemistry 2, p545-553 (2006)), Sinha et al. (“Inhibitors of plasma kallikrein” WO08/016883), Shigenaga et al. (“Plasma Kallikrein Inhibitors” WO2011/118672), and Kolte et al. (“Biochemical characterization of a novel high-affinity and specific kallikrein inhibitor”, British Journal of Pharmacology (2011), 162(7), 1639-1649). Also, Steinmetzer et al. (“Serine protease inhibitors” WO2012/004678) describes cyclized peptide analogs which are inhibitors of human plasmin and plasma kallikrein.

To date, no small molecule synthetic plasma kallikrein inhibitor has been approved for medical use. The molecules described in the known art suffer from limitations such as poor selectivity over related enzymes such as KLK1, thrombin and other serine proteases, and poor oral availability. Treatment with a large protein plasma kallikrein inhibitor has been shown to effectively treat HAE by preventing the release of bradykinin which causes increased vascular permeability (A. Lehmann “Ecallantide (DX-88), a plasma kallikrein inhibitor for the treatment of hereditary angioedema and the prevention of blood loss in on-pump cardiothoracic surgery” Expert Opin. Biol. Ther. 8, p1187-99). However, risks of anaphylactic reactions have been reported for Ecallantide.

Furthermore, many molecules in the known art feature a highly polar and ionisable guanidine or amidine functionality. It is well known that such functionalities may be limiting to gut permeability and therefore to oral availability. For example, it has been reported by Tamie J. Chilcote and Sukanto Sinha (“ASP-634: An Oral Drug Candidate for Diabetic MacularEdema”, ARVO 2012 May 6th-May 9, 2012, Fort Lauderdale, Florida, Presentation 2240) that ASP-440, a benzamidine, suffers from poor oral availability. It is further reported that absorption may be improved by creating a prodrug such as ASP-634. However, it is well known that prodrugs can suffer from several drawbacks, for example, poor chemical stability and potential toxicity from the inert carrier or from unexpected metabolites. In another report, indole amides are claimed as compounds that might overcome problems associated with drugs possessing poor or inadequate ADME-tox and physicochemical properties although no inhibition against plasma kallikrein is presented or claimed (Griffioen et al., “Indole amide derivatives and related compounds for use in the treatment of neurodegenerative diseases”, WO2010142801).

Other plasma kallikrein inhibitors known in the art are generally small molecules, some of which include highly polar and ionisable functional groups, such as guanidines or amidines. Recently, plasma kallikrein inhibitors that do not feature guanidine or amidine functionalities have been reported. For example Brandl et al. (“N-((6-amino-pyridin-3-yl)methyl)-heteroaryl-carboxamides as inhibitors of plasma kallikrein” WO2012/017020), Evans et al. (“Benzylamine derivatives as inhibitors of plasma kallikrein” WO2013/005045), Allan et al. (“Benzylamine derivatives” WO2014/108679), Davie et al. (“Heterocyclic derivates” WO2014/188211), Davie et al. (“N-((het)arylmethyl)-heteroaryl-carboxamides compounds as plasma kallikrein inhibitors” WO2016/083820).

The applicant has developed a novel series of compounds that are inhibitors of plasma kallikrein, which are disclosed in WO2016/083820 (PCT/GB2015/053615). These compounds demonstrate good selectivity for plasma kallikrein. One such compound is N-[(3-fluoro-4-methoxypyridin-2-yl)methyl]-3-(methoxymethyl)-1-({4-[(2-oxopyridin-1-yl)methyl]phenyl}methyl)pyrazole-4-carboxamide. Certain dosage forms comprising this compound are disclosed in Collett et al. WO2019/106361. The name N-[(3-fluoro-4-methoxypyridin-2-yl)methyl]-3-(methoxymethyl)-1-({4-[(2-oxopyridin-1-yl)methyl]phenyl}methyl)pyrazole-4-carboxamide denotes the structure depicted in Formula A.

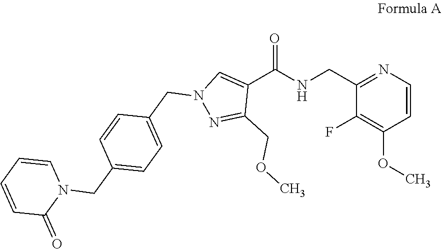 [SMILES: COCc1nn(Cc2ccc(Cn3ccccc3=O)cc2)cc1C(=O)NCc1nccc(OC)c1F]

There is therefore a need for a treatment of bradykinin-mediated angioedema (e.g. HAE or BK-AEnH) that is less burdensome on the patient to improve patient compliance. In particular, there is a need for a treatment of bradykinin-mediated angioedema (e.g. HAE or BK-AEnH) that can be administered orally. There is also a need for an oral treatment of acute bradykinin-mediated angioedema (e.g. HAE or BK-AEnH) attacks upon demand e.g. upon recognition of symptoms of an acute bradykinin-mediated angioedema (e.g. HAE or BK-AEnH) attack. In particular there is a need for an effective oral treatment in young patients (e.g. between the ages of 2 and less than 18). Existing injectable treatments can cause pain at the injection site, and the injection itself can cause the child stress. Moreover, there is a need for specific oral dosage forms that are more suitable for patients who may struggle to swallow tablets (e.g. children, the elderly, and patients suffering from a laryngeal attack).

### DESCRIPTION OF THE INVENTION

As noted above, there is a lack of approved oral treatments for bradykinin-mediated angioedema disorders such as HAE and BK-AEnH. To date, there are no authorised on-demand oral treatments of HAE, with all of the authorised treatments being injectable. HAE attacks resolve faster and are shorter after early treatment (Maurer M et al. PLoS ONE 2013;8(2): e53773. doi:10.1371/journal.pone.0053773) and thus early intervention when an attack is expected, or ongoing, is essential to desirably manage the disease. Injectable treatments suffer from late dosing because the patient may need to prepare the dosage form or even travel to hospital for treatment. Therefore, HAE treatment is often undermined by late dosing caused by the high burden on the patient. Indeed, Maurer M et al. explains that more than 60% of patients administer their HAE injectable more than one hour after the onset of an attack. Without wishing to be bound by theory, it is thought that HAE injectable treatments suffer from late dosing for reasons such as inconvenience (self-administration is not always possible e.g. in children), pain (both during and after the injection), and hope (rather than treat, patients frequently will just hope for a less severe attack). HAE drugs are often used off-label to treat some types of BK-AEnH, so the problems associated with HAE treatments apply similarly to treatment of BK-AEnH.

The present invention aims to solve this problem, and in particular, to provide treatments of bradykinin-mediated angioedema disorders such as HAE and BK-AEnH that are especially suitable for patients that require a dosage form that is easy to administer orally. Such patients include young patients (e.g. those aged between 2 and less than 18, particularly those aged between 2 and less than 12), elderly patients (e.g. those aged 70 years or older), patients that often suffer from bradykinin-mediated attacks that are laryngeal attacks, or those patients that regularly might not have fluid (e.g. water) readily available to aid the swallowing of an oral dosage form.

### Definitions

As used herein, and unless explicitly specified otherwise, the term “compound” or “compound of Formula A” is shorthand for “compound of Formula A (or a pharmaceutically acceptable salt and/or solvate thereof)”. The compound of Formula A is shown below and is named: N-[(3-fluoro-4-methoxypyridin-2-yl)methyl]-3-(methoxymethyl)-1-({4-[(2-oxopyridin-1-yl)methyl]phenyl}methyl)pyrazole-4-carboxamide.

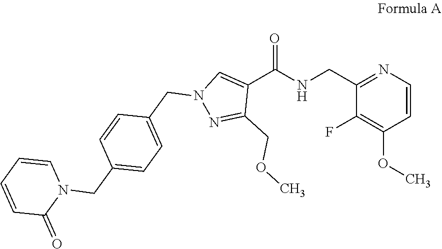 [SMILES: COCc1nn(Cc2ccc(Cn3ccccc3=O)cc2)cc1C(=O)NCc1nccc(OC)c1F]

It will be understood that “pharmaceutically acceptable salts and/or solvates thereof” means “pharmaceutically acceptable salts thereof”, “pharmaceutically acceptable solvates thereof”, and “pharmaceutically acceptable solvates of salts thereof”. The preferred form of the compound of formula A is the free base form, specifically the anhydrous free base form (when it is possible to keep the free base anhydrous).

The term “pharmaceutically acceptable salt” means a physiologically or toxicologically tolerable salt, which can be, for example, pharmaceutically acceptable acid addition salts. For instance, as the compound of Formula A contains a basic group, e.g. a basic nitrogen, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, pacetamidobenzoates, dihydroxybenzoates, hydroxynaphthoates, succinates, ascorbates, oleates, bisulfates and the like. The preferred pharmaceutically acceptable salt of the compound is the hydrochloride salt.

Hemisalts of acids can also be formed, for example, hemisulfate salts.

For a review of suitable salts, see “Handbook of Pharmaceutical Salts: Properties, Selection and Use” by Stahl and Wermuth (Wiley-VCH, Weinheim, Germany, 2002).

The term “solvate” is used herein to describe a molecular complex comprising the compound of the invention and a one or more pharmaceutically acceptable solvent molecules, for example, ethanol or water. The term “hydrate” is employed when the solvent is water and for the avoidance of any doubt, the term “hydrate” is encompassed by the term “solvate”.

The compound of Formula A may include compounds that differ only in the presence of one or more isotopically enriched atoms. For example, compounds wherein hydrogen is replaced by deuterium or tritium, or wherein carbon is replaced by ¹³C or ¹⁴C, are within the scope of the present invention.

The terms “acute attack of bradykinin-mediated angioedema”, “acute bradykinin-mediated angioedema attack”, “bradykinin-mediated angioedema attack”, or “attack of bradykinin-mediated angioedema” are used interchangeably herein. The “bradykinin-mediated angioedema” can be HAE or BK-AEnH. Preferably, the bradykinin-mediated angioedema is HAE, and when this is the case, the “acute attack of bradykinin-mediated angioedema” will be the “acute attack of HAE”, which can be used interchangeably with “acute HAE attack”, “HAE attack”, or “attack of HAE”.

The term “hereditary angioedema” (shortened to HAE) means any bradykinin-mediated angioedema caused by an inherited genetic dysfunction, fault, or mutation. As a result, the term “HAE”includes at least HAE type 1, HAE type 2, and normal C1 inhibitor HAE (normal C1-Inh HAE).

The term “bradykinin-mediated angioedema nonhereditary” or “BK-AEnH” is a bradykinin-mediated angioedema not caused by an inherited genetic dysfunction/fault/mutation i.e. it is not a hereditary angioedema (HAE). The underlying cause of the BK-AEnH can be unknown and/or undefined, but the signs and symptoms of BK-AEnH are similar to those of HAE, which without being bound by theory, is thought to be on account of the shared bradykinin-mediated pathway between HAE and BK-AEnH. Specific BK-AEnH that can be treated in accordance with the invention are selected from: non-hereditary angioedema with normal C1 Inhibitor (AE-nC1 Inh), which can be environmental, hormonal, or drug-induced; acquired angioedema; anaphylaxis associated angioedema; angiotensin converting enzyme (ACE or ace) inhibitor-induced angioedema; dipeptidyl peptidase-4inhibitor-induced angioedema; and tPA-induced angioedema (tissue plasminogen activator-induced angioedema).

The skilled person would understand “on-demand” treatment, in the context of bradykinin-mediated angioedema (BK-AEnH or preferably HAE), to mean that the compound of Formula A is administered upon need of therapy in connection with one specific bradykinin-mediated angioedema attack (a BK-AEnH attack or preferably an HAE attack). As described herein, this one specific bradykinin-mediated angioedema attack (a BK-AEnH attack or preferably an HAE attack) can be ongoing (e.g. treatment is initiated upon recognition of a symptom of a bradykinin-mediated angioedema attack) or likely to occur (e.g. when the patient anticipates that a bradykinin-mediated angioedema attack might be induced or triggered). Multiple dosage amounts of the compound of Formula A may be administered as part of the on-demand treatment, but these multiple dosages will be administered in connection with the same single bradykinin-mediated angioedema attack (a BK-AEnH attack or preferably an HAE attack). In other words, “on-demand” does not require the administration of the compound of Formula A continuously at regular intervals (e.g. once a week, twice a week, etc.) irrespective of an instance of a bradykinin-mediated angioedema attack (a BK-AEnH attack or preferably an HAE attack). In treatments of the invention, the compound of Formula A is taken when the patient requires fast-acting therapeutic effects. Particular “on-demand” treatments of the invention include: (i) treating a bradykinin-mediated angioedema attack (a BK-AEnH attack or preferably an HAE attack) on-demand, when the compound of Formula A is administered upon recognition of a symptom of a bradykinin-mediated angioedema attack (a BK-AEnH attack or preferably an HAE attack), and (ii) prophylactically reducing the likelihood of a bradykinin-mediated angioedema attack (a BK-AEnH attack or preferably an HAE attack) on-demand, e.g. when it is anticipated that a bradykinin-mediated angioedema attack (a BK-AEnH attack or preferably an HAE attack) attack might be induced (or triggered). These are discussed below in more detail.

In any of the treatments of the invention described herein, the patient is preferably a human. Bradykinin-mediated angioedema (e.g. BK-AEnH or preferably HAE) can affect patients of all ages. Accordingly, the human patient can be a child (ages 0 to less than 18 years) or an adult (18 years old or older). In any of the treatments described herein, the patient can have a predisposition to angioedema. Specifically, the patient can be aged 12 years and above. The patient can also be aged 2 years and above.

The human patient can be aged between 2 and less than 18. The human patient can be aged between 2 and less than 12. The human patient can be aged between 12 and less than 18. The patient can be at least 70 years old.

As demonstrated in the examples, the compound of Formula A is a potent inhibitor of plasma kallikrein. As already explained, inhibiting plasma kallikrein inhibits the cleavage of high molecular weight kininogen that contributes to a bradykinin-mediated angioedema attack (a BK-AEnH attack or preferably an HAE attack). Additionally, and as demonstrated in Example 4, the compound of Formula A is also capable of reducing the cleavage of plasma prekallikrein and the generation of Factor XIIa (FXIIa) following activation of the contact system. These advantageous additional effects support the treatment of the invention being highly efficacious and are demonstrated in particular when the concentration of the compound of Formula A is at least 500 ng/mL of plasma. A plasma concentration of at least 500 ng/mL can be observed following administration of a dosage amount of at least about 60 mg (more specifically, at least about 70 or about 80 mg) of the compound of Formula A.

Accordingly, in any of the treatments of the invention disclosed herein, particularly following a dosage amount of the compound of Formula A of at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg), in addition to inhibiting plasma kallikrein, the treatments can also reduce the cleavage of plasma prekallikrein to generate plasma kallikrein and/or reduce the generation of Factor XIIa (FXIIa) following administration. Thus, in some embodiments, particularly following a dosage amount of the compound of Formula A of at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg), the treatments can block the cleavage of plasma prekallikrein to generate plasma kallikrein and/or block the cleavage of FXII to generate FXIIa.

Treatments of Bradykinin-Mediated Angioedema (e.g. BK AEnH Attacks, Preferably HAE Attacks) Attacks On-Demand

As noted above, “bradykinin-mediated angioedema” can be selected from HAE and BK-AEnH. The bradykinin-mediated angioedema can be BK-AEnH. Preferably, the bradykinin-mediated angioedema is HAE.

The present invention provides a treatment of bradykinin-mediated angioedema that is improved compared to any bradykinin-mediated angioedema treatment currently available. The present invention provides an oral treatment of HAE that is particularly useful as an on-demand treatment of bradykinin-mediated angioedema attacks, and/or as an on-demand treatment to reduce the likelihood of bradykinin-mediated angioedema attacks. Specifically, as described herein, the treatments according to the invention (i) have a rapid onset of action, (ii) are potent, (iii) have a good safety profile, and (iv) have prolonged pharmacodynamic effects.

Thus, in accordance with the present invention, there is provided a method for treating bradykinin-mediated angioedema on-demand comprising: orally administering the compound of Formula A (or a pharmaceutically acceptable salt and/or solvate thereof) to a patient in need thereof on-demand, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet.

There is provided the compound of Formula A (or a pharmaceutically acceptable salt and/or solvate thereof) for use in treating bradykinin-mediated angioedema on-demand comprising: orally administering the compound of Formula A (or a pharmaceutically acceptable salt and/or solvate thereof) to a patient in need thereof on-demand, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet.

In accordance with an aspect of the invention there is provided a method for treating an attack of bradykinin-mediated angioedema on-demand comprising: orally administering the compound of Formula A to a patient in need thereof, wherein the compound of Formula A is orally administered on-demand upon recognition of a symptom of an bradykinin-mediated angioedema attack, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet.

Thus, an aspect of the invention provides the compound of Formula A for use in treating an acute attack of bradykinin-mediated angioedema on-demand comprising: orally administering the compound of Formula A to a patient in need thereof, wherein the compound of Formula A is orally administered on-demand upon recognition of a symptom of a bradykinin-mediated angioedema attack, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet.

When the bradykinin-mediated angioedema is HAE, the “bradykinin-mediated angioedema attack” is an “HAE attack”. When the bradykinin-mediated angioedema is BK-AEnH, the “bradykinin-mediated angioedema attack” is a “BK-AEnH attack” As noted above, the bradykinin-mediated angioedema is preferably HAE.

Although each bradykinin-mediated angioedema attack (e.g. a BK-AEnH attack, preferably an HAE attack) can be different in severity and in terms of the area affected, patients who suffer from bradykinin-mediated angioedema (e.g. BK-AEnH, preferably HAE), medical professionals with knowledge of bradykinin-mediated angioedema (e.g. BK-AEnH, preferably HAE), and carers of bradykinin-mediated angioedema (e.g. BK-AEnH, preferably HAE) patients are astute in identifying symptoms of a bradykinin-mediated angioedema attack (e.g. a BK-AEnH attack, preferably an HAE attack). These symptoms include, but are not limited to: swelling of tissues such as in the hands, feet, limbs, face, intestinal tract, and/or airway; fatigue; headache; muscle aches; skin tingling; abdominal pain; nausea; vomiting; diarrhoea; difficulty swallowing; hoarseness; shortness of breath; and/or mood changes. Thus, in some embodiments, administration of the compound of Formula A can occur upon recognition of at least one of the above symptoms.

The treatments of the invention can treat patients suffering from a laryngeal attack.

The skilled person would also understand that “administered upon recognition of a symptom of a bradykinin-mediated angioedema attack (e.g. a BK-AEnH attack, preferably an HAE attack)” means that administration occurs as quickly as feasibly possible after the symptom of a bradykinin-mediated angioedema attack (e.g. a BK-AEnH attack, preferably an HAE attack) is recognised. For example, patients are expected to have the compound of Formula A easily and readily available at all times (most likely in the form of a pharmaceutically acceptable composition) to ensure that treatment can occur upon recognition of a symptom of a bradykinin-mediated angioedema attack (e.g. a BK-AEnH attack, preferably an HAE attack). In other words, the treatment occurs on-demand. For example, in some embodiments, the compound of Formula A can be administered within 1 hour of the symptom of a bradykinin-mediated angioedema attack (e.g. a BK-AEnH attack, preferably an HAE attack) being recognised, preferably within 30 minutes, within 20 minutes, within 10 minutes, or within 5 minutes of the symptom of a bradykinin-mediated angioedema attack (e.g. a BK-AEnH attack, preferably an HAE attack) being recognised.

If the symptom of a bradykinin-mediated angioedema attack (e.g. a BK-AEnH attack, preferably an HAE attack) is recognised in the prodromal phase, an embodiment of the invention is that the compound of Formula A can be administered in the prodromal phase of a bradykinin-mediated angioedema attack (e.g. a BK-AEnH attack, preferably an HAE attack)attack. In these circumstances, the symptom recognised can be a slight swelling, in particular, a slight swelling affecting the face and neck. In addition, or in the alternative, the symptom can be abdominal pain, in particular, abdominal pain is considered to be characteristic of a bradykinin-mediated angioedema attack (e.g. a BK-AEnH attack, preferably an HAE attack). In addition, or in the alternative, the symptom can be a reddening of the skin such as erythema marginatum.

Treatment in accordance with the invention can prevent a bradykinin-mediated angioedema attack (e.g. a BK-AEnH attack, preferably an HAE attack) from increasing in severity. In some circumstances, treatment can shorten the attack duration, and sometimes even halt the attack in its entirety. For instance, treatment can halt the progression of a peripheral bradykinin-mediated angioedema attack (e.g. a BK-AEnH attack, preferably an HAE attack) or an abdominal bradykinin-mediated angioedema attack (e.g. a BK-AEnH attack, preferably an HAE attack). In some embodiments, treatment according to the invention can suppress the subsequent onset of swelling, sometimes completely, and in particular when treatment is initiated in the prodromal phase. In particular, in some embodiments, the bradykinin-mediated angioedema attack (e.g. a BK-AEnH attack, preferably an HAE attack) can be prevented from progressing into the swelling stage when the treatment is initiated in the prodromal phase.

The compound of Formula A can be sufficient for treating the bradykinin-mediated angioedema attack (e.g. the BK-AEnH attack, preferably the HAE attack) alone i.e. without the patient being administered any active pharmaceutical ingredient other than the compound of Formula A. Thus, in some embodiments of the invention, no active pharmaceutical ingredient other than the compound of Formula A is administered to the patient in order to treat the bradykinin-mediated angioedema attack (e.g. the BK-AEnH attack, preferably the HAE attack). In particular, in some embodiments, the treatments of the invention do not require administering any active pharmaceutical ingredient for treating a bradykinin-mediated angioedema attack (e.g. a BK-AEnH attack, preferably an HAE attack) other than the compound of Formula A (e.g. a rescue medication such as pdC1INH, rhC1INH, or icatibant is not required). More specifically, in some embodiments, no active pharmaceutical ingredient for treating a bradykinin-mediated angioedema attack (e.g. a BK-AEnH attack, preferably an HAE attack), other than the compound of Formula A is administered to the patient (e.g. a rescue medication such as pdC1INH, rhC1INH, or icatibant is not required).

Alternatively, in some embodiments, the treatments of the invention may be used in combination with other treatments of bradykinin-mediated angioedema (e.g. BK-AEnH, preferably HAE). For example, in some embodiments, the on-demand acute therapy described herein can be used as a “top-up” to another treatment of bradykinin-mediated angioedema (e.g. BK-AEnH, preferably HAE). In some embodiments, the patient may be taking a prophylactic treatment of bradykinin-mediated angioedema (e.g. BK-AEnH, preferably HAE) and might use the on-demand treatments described herein to treat a bradykinin-mediated angioedema attack (e.g. a BK-AEnH attack, preferably an HAE attack) that was not prevented by the other prophylactic treatment of bradykinin-mediated angioedema attack (e.g. a BK-AEnH attack, preferably an HAE attack).

For instance, in some embodiments, a method for treating bradykinin-mediated angioedema (e.g. BK-AEnH, preferably HAE) in a patient already taking a C1 inhibitor (such as Cinryze®, Haegarda®, Berinert®) for prophylaxis is provided comprising: orally administering the compound of Formula A to the patient on-demand upon recognition of a symptom of a bradykinin-mediated angioedema attack (e.g. a BK-AEnH attack, preferably an HAE attack). In another embodiment, a method for treating bradykinin-mediated angioedema (e.g. BK-AEnH, preferably HAE) in a patient already taking lanadelumab for prophylaxis is provided comprising: orally administering the compound of Formula A to the patient on-demand upon recognition of a symptom of a bradykinin-mediated angioedema attack (e.g. a BK-AEnH attack, preferably an HAE attack). In another embodiment, a method for treating bradykinin-mediated angioedema (e.g. BK-AEnH, preferably HAE) in a patient already taking berotralstat for prophylaxis is provided comprising: orally administering the compound of Formula A to the patient on-demand upon recognition of a symptom of a bradykinin-mediated angioedema attack (e.g. a BK-AEnH attack, preferably an HAE attack).

In any of the above treatments, the symptom can be recognised by the patient. In any of the above treatments, the symptom can be recognised by a medical professional such as a medical professional with knowledge of bradykinin-mediated angioedema (e.g. BK-AEnH, preferably HAE). In any of the above treatments, the symptom can be recognised by a carer of the patient.

Treatments according to the invention can reduce the proportion of bradykinin-mediated angioedema attacks (e.g. BK-AEnH attacks, preferably HAE attacks) that progress by one level or more on a 5-point Likert scale (5LS). Treatments according to the invention can reduce the proportion of bradykinin-mediated angioedema attacks (e.g. BK-AEnH attacks, preferably HAE attacks) that progress by one level or more on a 5LS within 12 hours of administering the compound. Treatments according to the invention can improve the resolution time of a bradykinin-mediated angioedema attack (e.g. a BK-AEnH attack, preferably a HAE attack) to “none” on a 5LS. 5LS is a known scale in the art (see e.g. Allergy Asthma Proc. 2018 Jan. 1; 39(1):74-80. doi: 10.2500/aap.2018.39.4095) that can be used to report the severity of bradykinin-mediated angioedema attacks (e.g. BK-AEnH attacks, preferably HAE attacks) and for example can be used to report attacks as “none”, “mild”, “moderate”, “severe” or “very severe”.

Treatments according to the invention can reduce the proportion of bradykinin-mediated angioedema attacks (e.g. BK-AEnH attacks, preferably HAE attacks) that are rated “worse” or “much worse” on a 7-point transition question (7TQ). Treatments according to the invention can increase the proportion of bradykinin-mediated angioedema attacks (e.g. BK-AEnH attacks, preferably HAE attacks) that are rated as “better” or “much better”. 7TQ is a known index in the art that can be used to score the progression of a bradykinin-mediated angioedema attack (e.g. a BK-AEnH attack, preferably a HAE attack) and to report attacks as “much better”, “better”, “a little better”, “no change”, “a little worse”, “worse”, or “much worse”.

In some embodiments of any of the on-demand treatments of a bradykinin-mediated angioedema attack (e.g. a BK-AEnH attack, preferably an HAE attack) of the invention, the patient can be administered a single dosage amount of the compound of Formula A to treat the bradykinin-mediated angioedema attack (e.g. a BK-AEnH attack, preferably an HAE attack). In some other embodiments of any of the on-demand treatments of bradykinin-mediated angioedema attacks (e.g. BK-AEnH attacks, preferably HAE attacks) of the invention, the patient can be administered multiple dosage amounts of the compound of Formula A to treat the acute bradykinin-mediated angioedema attack (e.g. a BK-AEnH attack, preferably an HAE attack). For example, the on-demand treatment can comprise administering two dosage amounts of the compound of Formula A within a 24 hour period starting from the time of taking the first dosage amount. Alternatively, the on-demand treatment can comprise administering three dosage amounts of the compound of Formula A within a 24 hour period starting from the time of taking the first dosage amount. Alternatively, the on-demand treatment can comprise administering four dosage amounts of the compound of Formula A within a 24 hour period starting from the time of taking the first dosage amount. When multiple dosage amounts are taken, the dosage amount can be evenly spaced apart such that there is an approximately equal time period between each dosage amount e.g. taking the subsequent dosage amount at 8 hours, 16 hours and 24 hours following the first dosage amount.

In some embodiments of any of the on-demand treatments of a bradykinin-mediated angioedema attack (e.g. a BK-AEnH attack, preferably an HAE attack) of the invention, the patient can be administered the daily dosage amount in two dosage amounts per day. These two dosage amounts can be administered simultaneously, separately or sequentially. In some embodiments, the two dosage amounts can be administered at any time within the day, with the interval between the two dosage amounts being specific to the patient, and the severity of the bradykinin-mediated angioedema attack (e.g. the BK-AEnH attack, preferably the HAE attack). In some embodiments, the second dosage amount can be administered within about 2 hours of the first (more specifically, between about 1 and 2 hours following the first dosage amount). In some embodiments, the second dosage amount can be administered between about 1 and about 4 hours of the first (more specifically, between about 1 and 3 hours, about 2 and 3 hours, or between 3 hours and about 4 hours, following the first dosage amount). In some embodiments, the second dosage amount can be administered between about 4 and about 12 hours of the first (more specifically, between about 4 and about 8 hours, or at about 6 hours, following the first dosage amount). In some embodiments, the second dosage amount can be administered between about 2 and about 6 hours of the first (more specifically, between about 3 and about 6 hours, following the first dosage amount). In some embodiments, the second dosage amount can be administered within about 8 hours of the first (more specifically, between about 4 and about 8 hours following the first dosage amount). In some embodiments, the second dosage amount can be administered within about 12 hours of the first (more specifically, between about 8 and about 12 hours following the first dosage amount). In some embodiments, the second dosage amount can be administered within about 16 hours of the first (more specifically, between about 12 and about 16 hours following the first dosage amount). In some embodiments, the second dosage amount can be administered within about 20 hours of the first (more specifically, between about 16 and about 20 hours following the first dosage amount). In some embodiments, the second dosage amount can be administered within about 24 hours of the first (more specifically, between about 20 and about 24 hours following the first dosage amount). In these embodiments, each of the two dosage amounts can be 600 mg of the compound of Formula A.

In any of the on-demand treatments of a bradykinin-mediated angioedema attack (e.g. a BK-AEnH attack, preferably an HAE attack) of the invention, the patient can be administered the daily dosage amount in two dosage amounts per day, wherein the second dosage amount can be administered at least about 6 hours after the first dosage amount. The patient can be administered the daily dosage amount in two dosage amounts per day, wherein the second dosage amount can be administered between about 5 and about 7 hours after the first dosage amount. More specifically, the patient can be administered the daily dosage amount in two dosage amounts per day, wherein the second dosage amount can be administered about 6 hours after the first dosage amount. In these embodiments, each of the two dosage amounts can be 600 mg of the compound of Formula A. Each of these 600 mg dosage amounts can be two tablets comprising 300 mg of the compound of Formula A.

In some embodiments of any of the on-demand treatments of a bradykinin-mediated angioedema attack (e.g. a BK-AEnH attack, preferably an HAE attack) of the invention, the patient can be administered the daily dosage amount in three dosage amounts per day. These three dosage amounts can be administered simultaneously, separately or sequentially. In some embodiments, the three dosage amounts can be administered at any time within the day, with the interval between the three dosage amounts being specific to the patient, and the severity of the bradykinin-mediated angioedema attack (e.g. the BK-AEnH attack, preferably the HAE attack). In some embodiments, the second and third dosage amounts can be both administered within about 4 hours of the first. More specifically, the second dosage amount can be administered between about 1 and 3 hours following the first dosage amount and the third dosage amount can be administered between about 3 and about 4 hours following the first dosage amount. The second dosage amount can be administered between about 4 and about 12 hours of the first (more specifically, between about 4 and about 8 hours, or at about 6 hours, following the first dosage amount), and the third dosage amount can be administered between about 4 and about 12 hours of the second (more specifically, between about 4 and about 8 hours, or at about 6 hours, following the second dosage amount). Even more specifically, the second dosage amount can be administered about 2 hours following the first dosage amount and the third dosage amount can be administered about 4 hours following the first dosage amount. In some embodiments, the second and third dosage amounts can both be administered within about 8 hours of the first. More specifically, the second dosage amount can be administered between about 3 and 5 hours of the first dosage amount and the third dosage amount can be administered between about 7 and about 8 hours following the first dosage amount. Even more specifically, the second dosage amount can be administered about 4 hours following the first dosage amount and the third dosage amount can be administered about 8 hours following the first dosage amount. In some embodiments, the second and third dosage amounts can both be administered within about 16 hours of the first. More specifically, the second dosage amount can be administered between about 7 and 9 hours of the first dosage amount and the third dosage amount can be administered between about 15 and about 16 hours following the first dosage amount. Even more specifically, the second dosage amount can be administered about 8 hours following the first dosage amount and the third dosage amount can be administered about 16 hours following the first dosage amount. In these embodiments, each of the three dosage amounts can be 600 mg of the compound of Formula A.

In any of the on-demand treatments of bradykinin-mediated angioedema attacks (e.g. BK-AEnH attacks, preferably HAE attacks) of the invention, the patient can be administered the daily dosage amount in three dosage amounts per day, wherein the second and third dosage amounts can be administered at least about 6 hours after the preceding dosage amount. The patient can be administered the daily dosage amount in three dosage amounts per day, wherein the second dosage amount can be administered between about 5 and about 7 hours after the first dosage amount, and the third dosage amount can be administered between about 11 and about 13 hours after the first dosage amount. More specifically, the patient can be administered the daily dosage amount in three dosage amounts per day, wherein the second dosage amount can be administered about 6 hours after the first dosage amount and the third dosage amount can be administered about 12 hours after the first dosage amount. In these embodiments, each of the three dosage amounts can be 600 mg of the compound of Formula A. Each of these 600 mg dosage amounts can be two tablets comprising 300 mg of the compound of Formula A.

Multiple dosage amounts can be administered if, for example, a bradykinin-mediated angioedema attack (e.g. a BK-AEnH attack, preferably an HAE attack) persists after administration of the first dosage amount. When used in this context, “persists” can mean that, e.g., the first dosage amount does not prevent a bradykinin-mediated angioedema attack (e.g. a BK-AEnH attack, preferably an HAE attack) from increasing in severity, or that the first dosage amount does not halt the bradykinin-mediated angioedema attack (e.g. the BK-AEnH attack, preferably the HAE attack) in its entirety, or that the first dosage amount does not decrease the severity of the bradykinin-mediated angioedema attack (e.g. the BK-AEnH attack, preferably the HAE attack). Accordingly, on-demand treatments of a bradykinin-mediated angioedema attack (e.g. a BK-AEnH attack, preferably an HAE attack) of the invention can comprise administering a first dosage amount, and then administering a second dosage amount if the bradykinin-mediated angioedema attack (e.g. the BK-AEnH attack, preferably the HAE attack) persists after administering the first dosage amount. On-demand treatments of a bradykinin-mediated angioedema attack (e.g. a BK-AEnH attack, preferably an HAE attack) of the invention can also comprise administering a first dosage amount, and then administering a second dosage amount if the bradykinin-mediated angioedema attack (e.g. the BK-AEnH attack, preferably the HAE attack) persists after administering the first dosage amount, and then administering a third dosage amount if the bradykinin-mediated angioedema attack (e.g. the BK-AEnH attack, preferably the HAE attack) persists after administering the second dosage amount. In each case, each subsequent dosage amount can be administered simultaneously, separately or sequentially. In each case, each subsequent dosage amount can be administered at least about 6 hours (e.g. at about 6 hours) after the preceding dosage amount. In each case, each dosage amount can comprise 600 mg of the compound, e.g., administered as two tablets comprising 300 mg.

Specifically, the on-demand treatments of a bradykinin-mediated angioedema attack (e.g. a BK-AEnH attack, preferably an HAE attack) of the invention can comprise administering a first dosage amount comprising 600 mg of the compound (e.g. as two tablets comprising 300 mg of the compound), and then administering a second dosage amount comprising 600 mg of the compound (e.g. as two tablets comprising 300 mg of the compound) if the a bradykinin-mediated angioedema attack (e.g. the BK-AEnH attack, preferably the HAE attack) persists after administering the first dosage amount. The second dosage amount can be administered at least about 6 hours (e.g. at about 6 hours) after the first dosage amount. If the bradykinin-mediated angioedema attack (e.g. the BK-AEnH attack, preferably the HAE attack) persists after the second dosage amount, the on-demand treatments of a bradykinin-mediated angioedema attack (e.g. a BK-AEnH attack, preferably an HAE attack) of the invention can comprise administering a third dosage amount comprising 600 mg of the compound (e.g. as two tablets comprising 300 mg of the compound). The third dosage amount can be administered at least about 6 hours (e.g. at about 6 hours) after the second dosage amount.

Multiple dosage amounts can be administered even if the severity of the bradykinin-mediated angioedema attack (e.g. the BK-AEnH attack, preferably the HAE attack) appears to have been reduced (or even halted in its entirety) after administration of the first dosage amount to prevent the bradykinin-mediated angioedema attack (e.g. the BK-AEnH attack, preferably the HAE attack) from increasing in severity again. For example, multiple dosage amounts can be used for the peace of mind of the patient e.g. to ease anxiety of the patient. Accordingly, on-demand treatments of a bradykinin-mediated angioedema attack (e.g. a BK-AEnH attack, preferably a HAE attack) of the invention can comprise administering a first dosage amount, and then administering a second dosage amount even if the severity of a bradykinin-mediated angioedema attack (e.g. a BK-AEnH attack, preferably a HAE attack) appears to have been reduced (or even halted in its entirety) after administration of the first dosage amount to prevent the bradykinin-mediated angioedema attack (e.g. the BK-AEnH attack, preferably the HAE attack) from increasing in severity again. Even if the severity of the bradykinin-mediated angioedema attack (e.g. the BK-AEnH attack, preferably the HAE attack) appears to have been reduced (or even halted in its entirety) after administration of the first and/or second dosage amount, on-demand treatments of a bradykinin-mediated angioedema attack (e.g. a BK-AEnH attack, preferably a HAE attack) of the invention can also comprise administering a third dosage amount to prevent the bradykinin-mediated angioedema attack (e.g. the BK-AEnH attack, preferably the HAE attack) from increasing in severity again. In each case, each subsequent dosage amount can be administered simultaneously, separately or sequentially. In each case, each subsequent dosage amount can be administered at least about 6 hours (e.g. at about 6 hours) after the preceding dosage amount. In each case, each dosage amount can comprise 600 mg of the compound, e.g., administered as two tablets comprising 300 mg of the compound.

Specifically, the on-demand treatments of a bradykinin-mediated angioedema attack (e.g. a BK-AEnH attack, preferably an HAE attack) of the invention can comprise administering a first dosage amount comprising 600 mg of the compound (e.g. as two tablets comprising 300 mg of the compound), and then administering a second dosage amount comprising 600 mg of the compound (e.g. as two tablets comprising 300 mg of the compound) even if the severity of the bradykinin-mediated angioedema attack (e.g. the BK-AEnH attack, preferably the HAE attack) appears to have been reduced (or even halted in its entirety) after administration of the first dosage amount to prevent the bradykinin-mediated angioedema attack (e.g. the BK-AEnH attack, preferably the HAE attack) from increasing in severity again. The second dosage amount can be administered at least about 6 hours (e.g. at about 6 hours) after the first dosage amount. Even if the severity of the bradykinin-mediated angioedema attack (e.g. the BK-AEnH attack, preferably the HAE attack) appears to have been reduced (or even halted in its entirety) after administration of the first and/or second dosage amount, the on-demand treatments of a bradykinin-mediated angioedema attack (e.g. a BK-AEnH attack, preferably a HAE attack) of the invention can comprise administering a third dosage amount comprising 600 mg of the compound (e.g. as two tablets comprising 300 mg of the compound) to prevent the bradykinin-mediated angioedema attack (e.g. the BK-AEnH attack, preferably the HAE attack) from increasing in severity again. The third dosage amount can be administered at least about 6 hours (e.g. at about 6 hours) after the second dosage amount.

The on-demand treatments of a bradykinin-mediated angioedema attack (e.g. a BK-AEnH attack, preferably an HAE attack) of the invention can comprise not administering more than three dosage amounts in a 24 hour period (e.g. three dosage amounts comprising 600 mg of the compound, optionally as 6 tablets each comprising 300 mg of the compound).

On-Demand Prophylactic Treatment of Bradykinin Mediated Angioedema Attack (e.g. BK AEnH Attacks, Preferably HAE Attacks)

In accordance with an aspect of the invention, there is provided a method for treating bradykinin-mediated angioedema (e.g. BK-AEnH, preferably HAE) on-demand comprising: orally administering the compound of Formula A to a patient in need thereof, wherein the compound of Formula A is orally administered on-demand to prophylactically reduce the likelihood of a bradykinin-mediated angioedema attack (e.g. a BK-AEnH attack, preferably an HAE attack), wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet.

Thus, an aspect of the invention provides the compound of Formula A for use in treating bradykinin-mediated angioedema (e.g. BK-AEnH, preferably HAE) comprising: orally administering the compound of Formula A to a patient in need thereof, wherein the compound of Formula A is orally administered on-demand to prophylactically reduce the likelihood of a bradykinin-mediated angioedema attack (e.g. a BK-AEnH attack, preferably an HAE attack), wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet.

In some embodiments, the compound of Formula A can be administered to prevent a bradykinin-mediated angioedema attack (e.g. a BK-AEnH attack, preferably an HAE attack). As noted above, preferably the bradykinin-mediated angioedema is HAE.

As discussed above, treatments in accordance with the invention do not require dosing of the compound of Formula A at regular intervals to provide prophylactic therapy. Indeed, in some embodiments, the compound of Formula A can be administered on-demand. For example, the compound of Formula A can be administered on-demand to reduce the likelihood of a bradykinin-mediated angioedema attack (e.g. a BK-AEnH attack, preferably an HAE attack), e.g. to prevent a bradykinin-mediated angioedema attack (such as a BK-AEnH attack, preferably an HAE attack) when it is anticipated that a bradykinin-mediated angioedema attack (e.g. a BK-AEnH attack, preferably an HAE attack) will be induced (or triggered) i.e. it is anticipated that the patient will suffer from a bradykinin-mediated angioedema attack (e.g. a BK-AEnH attack, preferably an HAE attack). In some embodiments, the patient can anticipate than a bradykinin-mediated angioedema attack (e.g. a BK-AEnH attack, preferably an HAE attack) will be induced (or triggered). In some embodiments, a medical professional such as a medical professional with knowledge of bradykinin-mediated angioedema (e.g. BK-AEnH, preferably HAE) can anticipate than a bradykinin-mediated angioedema attack (e.g. a BK-AEnH attack, preferably an HAE attack) will be induced (or triggered). In some embodiments, a carer for the patient can anticipate than a bradykinin-mediated angioedema attack (e.g. a BK-AEnH attack, preferably an HAE attack) will be induced (or triggered). For example, a bradykinin-mediated angioedema attack (e.g. a BK-AEnH attack, preferably an HAE attack) can be induced (or triggered) by various stimuli such as physical traumata (e.g. medical, dental or surgical procedures) and/or stress (e.g. high stress situations such as mental stress, which in some instances can be associated with taking examinations or mental stress associated with a medical, dental or surgical procedure). For example, a bradykinin-mediated angioedema attack (e.g. a BK-AEnH attack, preferably an HAE attack) can be induced (or triggered) by the elevated stress/anxiety levels of the patient when the patient might expect to have a bradykinin-mediated angioedema attack (e.g. a BK-AEnH attack, preferably an HAE attack). Additionally, the frequency of bradykinin-mediated angioedema attacks (e.g. BK-AEnH attacks, preferably HAE attacks) instances can vary over time in the same patient. Patients can often suffer from periods where the frequency of instances of bradykinin-mediated angioedema attacks (e.g. BK-AEnH attacks, preferably HAE attacks) is greater than normal. So, a bradykinin-mediated angioedema attack (e.g. a BK-AEnH attack, preferably an HAE attack) can be anticipated during periods where the patient is suffering from more frequent instances of bradykinin-mediated angioedema attacks (e.g. BK-AEnH attacks, preferably HAE attacks) compared to normal. Those familiar with bradykinin-mediated angioedema (e.g. BK-AEnH, preferably HAE) will be aware that bradykinin-mediated angioedema attacks (e.g. BK-AEnH attacks, preferably HAE attacks) can be induced (or triggered) in this way. Patients, medical professionals with knowledge of bradykinin-mediated angioedema (e.g. BK-AEnH, preferably HAE), and carers of patients can also be astute in anticipating such triggers. Thus, in accordance with the invention, the treatment can be administered on-demand when it is anticipated that the patient will be subjected to one or more of these stimuli or circumstances.

As discussed above, the patient can be administered the compound of Formula A as part of an on-demand prophylactic treatment of a bradykinin-mediated angioedema attack (e.g. a BK-AEnH attack, preferably an HAE attack). As discussed above, this treatment reduces the likelihood of a bradykinin-mediated angioedema attack (e.g. a BK-AEnH attack, preferably an HAE attack). However, in some circumstances, a patient can still suffer from a bradykinin-mediated angioedema attack (e.g. a BK-AEnH attack, preferably an HAE attack). Thus, an embodiment of the invention is that the patient can be administered the compound of Formula A as part of an on-demand prophylactic treatment of a bradykinin-mediated angioedema attack (e.g. a BK-AEnH attack, preferably an HAE attack), as discussed above, further comprising taking an on-demand dosage amount of the compound of Formula A upon recognition of a symptom of a bradykinin-mediated angioedema attack (e.g. a BK-AEnH attack, preferably an HAE attack) to treat a bradykinin-mediated angioedema attack (e.g. a BK-AEnH attack, preferably an HAE attack) should it arise. These on-demand treatments of bradykinin-mediated angioedema attacks (e.g. BK-AEnH attacks, preferably HAE attacks) are discussed above.

Thus, in some embodiments, there is provided a method for treating bradykinin-mediated angioedema (e.g. BK-AEnH, preferably HAE) on-demand comprising: orally administering the compound of Formula A to a patient in need thereof, wherein the compound of Formula A is orally administered on-demand to prophylactically reduce the likelihood of a bradykinin-mediated angioedema attack (e.g. a BK-AEnH attack, preferably an HAE attack), further comprising orally administering the compound of Formula A on-demand upon recognition of a symptom of an acute HAE attack, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet.

In some embodiments of any of the on-demand treatments of a bradykinin-mediated angioedema attack (e.g. a BK-AEnH attack, preferably an HAE attack) of the invention, the patient can be administered a single dosage amount of the compound of Formula A to treat the bradykinin-mediated angioedema attack (e.g. the BK-AEnH attack, preferably the HAE attack). In some other embodiments of any of the on-demand treatments of bradykinin-mediated angioedema attacks (e.g. BK-AEnH attacks, preferably HAE attacks) of the invention, the patient can be administered multiple dosage amounts of the compound of Formula A to treat the bradykinin-mediated angioedema attack (e.g. the BK-AEnH attack, preferably the HAE attack). For example, the on-demand treatment can comprise administering two dosage amounts of the compound of Formula A within a 24 hour period starting from the time of taking the first dosage amount. Alternatively, the on-demand treatment can comprise administering three dosage amounts of the compound of Formula A within a 24 hour period starting from the time of taking the first dosage amount. Alternatively, the on-demand treatment can comprise administering four dosage amounts of the compound of Formula A within a 24 hour period starting from the time of taking the first dosage amount. When multiple dosage amounts are taken, the dosage amount can be evenly spaced apart such that there is an approximately equal time period between each dosage amount e.g. taking the subsequent dosage amount at 8 hours, 16 hours and 24 hours following the initial dosage amount.

In some embodiments of any of the on-demand prophylactic treatments of bradykinin-mediated angioedema attacks (e.g. BK-AEnH attacks, preferably HAE attacks) described herein, the patient can be administered two dosage amounts per day. These two dosage amounts can be administered simultaneously, separately or sequentially. In some embodiments, the two dosage amounts can be administered at any time within the day, with the interval between the two dosage amounts being specific to the patient. In some embodiments, the second dosage amount can be administered within about 2 hours of the first (more specifically, between about 1 and 2 hours following the first dosage amount). In some embodiments, the second dosage amount can be administered between about 1 and about 4 hours of the first (more specifically, between about 1 and 3 hours, about 2 and 3 hours, or between 3 hours and about 4 hours, following the first dosage amount). In some embodiments, the second dosage amount can be administered between about 4 and about 12 hours of the first (more specifically, between about 4 and about 8 hours, or at about 6 hours, following the first dosage amount). In some embodiments, the second dosage amount can be administered between about 2 and about 6 hours of the first (more specifically, between about 3 and about 6 hours, following the first dosage amount). In some embodiments, the second dosage amount can be administered within about 8 hours of the first (more specifically, between about 4 and about 8 hours following the first dosage amount). In some embodiments, the second dosage amount can be administered within about 12 hours of the first (more specifically, between about 8 and about 12 hours following the first dosage amount). In some embodiments, the second dosage amount can be administered within about 16 hours of the first (more specifically, between about 12 and about 16 hours following the first dosage amount). In some embodiments, the second dosage amount can be administered within about 20 hours of the first (more specifically, between about 16 and about 20 hours following the first dosage amount). In some embodiments, the second dosage amount can be administered within about 24 hours of the first (more specifically, between about 20 and about 24 hours following the first dosage amount). In these embodiments, each of the two dosage amounts can be 600 mg of the compound of Formula A.

In any of the on-demand prophylactic treatments of bradykinin-mediated angioedema attacks (e.g. a BK-AEnH attack, preferably an HAE attack) described herein, the patient can be administered the daily dosage amount in two dosage amounts per day, wherein the second dosage amount can be administered at least about 6 hours after the first dosage amount. The patient can be administered the daily dosage amount in two dosage amounts per day, wherein the second dosage amount can be administered between about 5 and about 7 hours after the first dosage amount. More specifically, the patient can be administered the daily dosage amount in two dosage amounts per day, wherein the second dosage amount can be administered about 6 hours after the first dosage amount. In these embodiments, each of the two dosage amounts can be 600 mg of the compound of Formula A. Each of these 600 mg dosage amounts can be two tablets comprising 300 mg of the compound of Formula A.

In some embodiments of any of the on-demand prophylactic treatments of bradykinin-mediated angioedema attacks (e.g. BK-AEnH attacks, preferably HAE attack) described herein, the patient can be administered the daily dosage amount in three dosage amounts per day. These three dosage amounts can be administered simultaneously, separately or sequentially. In some embodiments, the three dosage amounts can be administered at any time within the day, with the interval between the three dosage amounts being specific to the patient. In some embodiments, the second and third dosage amounts can be both administered within about 4 hours of the first. More specifically, the second dosage amount can be administered between about 1 and 3 hours following the first dosage amount and the third dosage amount can be administered between about 3 and about 4 hours following the first dosage amount. The second dosage amount can be administered between about 4 and about 12 hours of the first (more specifically, between about 4 and about 8 hours, or at about 6 hours, following the first dosage amount), and the third dosage amount can be administered between about 4 and about 12 hours of the second (more specifically, between about 4 and about 8 hours, or at about 6 hours, following the second dosage amount). Even more specifically, the second dosage amount can be administered about 2 hours following the first dosage amount and the third dosage amount can be administered about 4 hours following the first dosage amount. In some embodiments, the second and third dosage amounts can both be administered within about 8 hours of the first. More specifically, the second dosage amount can be administered between about 3 and 5 hours of the first dosage amount and the third dosage amount can be administered between about 7 and about 8 hours following the first dosage amount. Even more specifically, the second dosage amount can be administered about 4 hours following the first dosage amount and the third dosage amount can be administered about 8 hours following the first dosage amount. In some embodiments, the second and third dosage amounts can both be administered within about 16 hours of the first. More specifically, the second dosage amount can be administered between about 7 and 9 hours of the first dosage amount and the third dosage amount can be administered between about 15 and about 16 hours following the first dosage amount. Even more specifically, the second dosage amount can be administered about 8 hours following the first dosage amount and the third dosage amount can be administered about 16 hours following the first dosage amount. In these embodiments, each of the three dosage amounts can be 600 mg of the compound of Formula A.

In any of the on-demand prophylactic treatments of bradykinin-mediated angioedema attacks (e.g. BK-AEnH attacks, preferably HAE attacks) described herein, the patient can be administered the daily dosage amount in three dosage amounts per day, wherein the second and third dosage amounts can be administered at least about 6 hours after the preceding dosage amount. The patient can be administered the daily dosage amount in three dosage amounts per day, wherein the second dosage amount can be administered between about 5 and about 7 hours after the first dosage amount, and the third dosage amount can be administered between about 11 and about 13 hours after the first dosage amount. More specifically, the patient can be administered the daily dosage amount in three dosage amounts per day, wherein the second dosage amount can be administered about 6 hours after the first dosage amount and the third dosage amount can be administered about 12 hours after the first dosage amount. In these embodiments, each of the three dosage amounts can be 600 mg of the compound of Formula A. Each of these 600 mg dosage amounts can be two tablets comprising 300 mg of the compound of Formula A.

Multiple dosage amounts can be administered if, for example, there is a continued need to prophylactically reduce the likelihood of a bradykinin-mediated angioedema attack (e.g. a BK-AEnH attack, preferably an HAE attack), e.g. if the patient continues to anticipate that a bradykinin-mediated angioedema attack (e.g. a BK-AEnH attack, preferably an HAE attack) might be induced, as discussed above. Accordingly, on-demand treatments of a bradykinin-mediated angioedema attack (e.g. a BK-AEnH attack, preferably an HAE attack) of the invention can comprise administering a first dosage amount, and then administering a second dosage amount if there is a continued need to prophylactically reduce the likelihood of a bradykinin-mediated angioedema attack (e.g. a BK-AEnH attack, preferably an HAE attack) after administering the first dosage amount. On-demand treatments of a bradykinin-mediated angioedema attack (e.g. a BK-AEnH attack, preferably an HAE attack) of the invention can also comprise administering a first dosage amount, and then administering a second dosage amount if there is a continued need to prophylactically reduce the likelihood of a bradykinin-mediated angioedema attack (e.g. a BK-AEnH attack, preferably an HAE attack) after administering the first dosage amount, and then administering a third dosage amount if there is a continued need to prophylactically reduce the likelihood of a bradykinin-mediated angioedema attack (e.g. a BK-AEnH attack, preferably an HAE attack) after administering the second dosage amount. In each case, each subsequent dosage amount can be administered simultaneously, separately or sequentially. In each case, each subsequent dosage amount can be administered at least about 6 hours (e.g. at about 6 hours) after the preceding dosage amount. In each case, each dosage amount can comprise 600 mg of the compound, e.g., administered as two tablets comprising 300 mg of the compound.

Specifically, the on-demand prophylactic treatments of bradykinin-mediated angioedema attacks (e.g. BK-AEnH attacks, preferably HAE attacks) described herein can comprise administering a first dosage amount comprising 600 mg of the compound (e.g. as two tablets each comprising 300 mg of the compound), and then administering a second dosage amount comprising 600 mg of the compound (e.g. as two tablets each comprising 300 mg of the compound) if there is a continued need to prophylactically reduce the likelihood of an acute HAE attack after administering the first dosage amount. The second dosage amount can be administered at least about 6 hours (e.g. at about 6 hours) after the first dosage amount. If there is a continued need to prophylactically reduce the likelihood of a bradykinin-mediated angioedema attack (e.g. a BK-AEnH attack, preferably an HAE attack) after the second dosage amount, the on-demand treatments of a bradykinin-mediated angioedema attack (e.g. a BK-AEnH attack, preferably an HAE attack) of the invention can comprise administering a third dosage amount comprising 600 mg of the compound (e.g. as two tablets each comprising 300 mg of the compound). The third dosage amount can be administered at least about 6 hours (e.g. at about 6 hours) after the second dosage amount.

The on-demand prophylactic treatments of bradykinin-mediated angioedema attacks (e.g. BK-AEnH attacks, preferably HAE attacks) described herein can comprise not administering more than three dosage amounts in a 24 hour period (e.g. three dosage amounts comprising 600 mg of the compound, optionally as 6 tablets each comprising 300 mg of the compound).

Continuous and Regular Prophylactic Treatment of Bradykinin Mediated Angioedema (e.g. BK AEnH, Preferably HAE)

In accordance with an aspect of the invention, there is provided a method for treating bradykinin-mediated angioedema (e.g. BK-AEnH, preferably HAE) comprising: orally administering the compound of Formula A to a patient in need thereof, wherein the compound of Formula A is orally administered to prophylactically reduce the likelihood of a bradykinin-mediated angioedema attack (e.g. a BK-AEnH attack, preferably an HAE attack), wherein the compound of Formula A is administered regularly to the patient, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet.

Thus, an aspect of the invention provides the compound of Formula A for use in treating bradykinin-mediated angioedema (e.g. BK-AEnH, preferably HAE) comprising: orally administering the compound of Formula A to a patient in need thereof, wherein the compound of Formula A is orally administered to reduce the likelihood of a bradykinin-mediated angioedema attack (e.g. a BK-AEnH attack, preferably an HAE attack), wherein the compound of Formula A is administered regularly to the patient, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet.

The term “administered regularly” is intended to mean administering the compound of Formula A continuously at regular intervals (e.g. once a week, twice a week, etc.) to provide an effective treatment. The healthcare professional would readily understand what regular (or continuous) administration is intended to mean.

In some embodiments, the compound of Formula A can be administered to prevent a bradykinin-mediated angioedema attack (e.g. a BK-AEnH attack, preferably an HAE attack).

In some embodiments, the compound of Formula A can be orally administered once daily. In another embodiment, the compound of Formula A can be administered twice daily. In another embodiment, the compound of Formula A can be administered three times daily. In another embodiment, the compound of Formula A can be administered every other day.

As discussed above, the patient can be administered the compound of Formula A as part of a continuous and regular prophylactic treatment of bradykinin-mediated angioedema (e.g. BK-AEnH, preferably HAE). As discussed above, this treatment reduces the likelihood of a bradykinin-mediated angioedema attack (e.g. a BK-AEnH attack, preferably an HAE attack). However, in some circumstances, a patient can still suffer from a bradykinin-mediated angioedema attack (e.g. a BK-AEnH attack, preferably an HAE attack). Thus, an embodiment of the invention is that the patient can be administered the compound of Formula A as part of a continuous and regular prophylactic treatment of bradykinin-mediated angioedema (e.g. BK-AEnH, preferably HAE), as discussed above, further comprising taking an on-demand dosage amount of the compound of Formula A upon recognition of a symptom of a bradykinin-mediated angioedema attack (e.g. a BK-AEnH attack, preferably an HAE attack) to treat a bradykinin-mediated angioedema attack (e.g. a BK-AEnH attack, preferably an HAE attack) should it arise. These on-demand treatments of bradykinin-mediated angioedema attacks (e.g. BK-AEnH attacks, preferably an HAE attacks) are discussed above.

Thus, in some embodiments, there is provided a method for treating bradykinin-mediated angioedema (e.g. BK-AEnH, preferably HAE) comprising: orally administering the compound of Formula A to a patient in need thereof, wherein the compound of Formula A is orally administered to prophylactically reduce the likelihood of a bradykinin-mediated angioedema attack (e.g. a BK-AEnH attack, preferably an HAE attack), wherein the compound of Formula A is administered regularly to the patient, further comprising orally administering the compound of Formula A on-demand upon recognition of a symptom of a bradykinin-mediated angioedema attack (e.g. a BK-AEnH attack, preferably an HAE attack), wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet.

BK-AEnH

As noted above, the bradykinin-mediated angioedema can be BK-AEnH.

Where the BK-AEnH is dipeptidyl peptidase-4 inhibitor-induced angioedema, the BK-AEnH can be induced by the use of dipeptidyl peptidase-4 inhibitor as an antidiabetic drug. The BK-AEnH can be dipeptidyl peptidase-4 inhibitor-induced by sitagliptin, metformin, saxagliptin, linagliptin, empagliflozin, alogliptin, or pioglitazone.

Where the BK-AEnH is ace inhibitor-induced angioedema, the BK-AEnH can be ace inhibitor-induced by benazepril, captopril, enalapril, fosinopril, lisinopril, moexipril, perindopril, quinapril, ramipril, or trandolapril.

Where the BK-AEnH is tPA-induced angioedema, the BK-AEnH can be induced by thrombolytic therapy using a tissue plasminogen activator. The patient can be receiving thrombolytic therapy using a tissue plasminogen activator e.g. to treat an acute stroke such as an ischemic stroke.

Where the BK-AEnH is non-hereditary angioedema with normal C1 Inhibitor (AE-nC1 Inh) and is drug-induced (i.e. drug-induced AE-nC1 Inh), the BK-AEnH can be drug-induced by at least one of a nonsteroidal anti-inflammatory agent, a P-lactam antibiotic, and a non-P lactam antibiotic. The nonsteroidal anti-inflammatory agent can be at least one of aspirin, celecoxib, diclofenac, diflunisal, etodolac ibuprofen, indomethacin, ketoprofen, ketorolac, nabumetone, naproxen, oxaprozin, piroxicam, salsalate, sulindac, and tolmetin.

Where the BK-AEnH is non-hereditary angioedema with normal C1 Inhibitor (AE-nC1 Inh) and is drug-induced (i.e. drug-induced AE-nC1 Inh), the BK-AEnH can be induced by an angiotensin II receptor blocker (ARB). For instance, the BK-AEnH can be induced by azilsartan, candesartan, eprosartan, irbesartan, losartan, olmesartan, telmisartan, or valsartan.

Where the BK-AEnH is drug-induced AE-nC1 Inh, the BK-AEnH can be drug-induced by beta blockers.

Where the BK-AEnH is non-hereditary angioedema with normal C1 Inhibitor (AE-nC1 Inh) and is hormonal-induced, the AE-nC1 Inh can be hormonally-induced by a hormonal contraceptive. In some embodiments, the AE-nC1 Inh can be hormonally-induced by oestrogen. For instance, the patient can be a female and be taking oestrogen e.g. as a contraceptive.

Dosing

In any of the treatments of the invention described herein, the compound of Formula A is orally administered in a therapeutically effective amount.

In some embodiments, the compound of Formula A can be administered at a daily dosage amount of between about 5 mg and about 2000 mg per day. “Daily dosage amount” means the total amount administered in one day. More specifically, the compound of Formula A can be administered at a daily dosage amount of between about 100 mg and about 1500 mg, about 300 mg to about 1800 mg, about 100 mg and about 1400 mg, about 200 mg and about 1200 mg, about 300 mg and about 1200 mg, about 600 mg and about 1200 mg, about 450 mg and about 900 mg, about 500 mg and about 1000 mg, about 450 mg and about 600 mg, about 500 mg and about 700 mg (more specifically, 600 mg), about 800 mg and about 1000 mg per day, about 900 mg and about 1400 mg (more specifically 1200 mg), or about 900 mg and about 1200 mg. In a specific embodiment, the daily dosage amount is 300 mg. In another specific embodiment, the daily dosage amount is 600 mg. In another specific embodiment, the daily dosage amount is 900 mg. In another specific embodiment, the daily dosage amount is 1200 mg. In another specific embodiment the daily dosage amount is 1800 mg.

The daily dosage amount can be administered as one single dosage amount, or sub-divided into multiple dosage amounts for administration periodically during the day. In turn, each dosage amount can administered as a single dosage form, or sub-divided into multiple dosage forms. For example, a 1200 mg daily dosage amount can be administered as two sub-divided dosage amounts of 600 mg, where each of these sub-divided dosage amounts can be administered as two sub-divided dosage forms of 300 mg. Where multiple dosage amounts and multiple dosage forms are used, these can be administered simultaneously, separately or sequentially.

In some embodiments, each single unit dosage form comprising the compound of Formula A comprises between about 5 mg and about 1000 mg, about 50 mg to about 800 mg, about 100 mg to about 700 mg, about 200 mg to about 700 mg, about 300 mg to about 700 mg, or about 500 mg to about 700 mg of the compound of Formula A. In some embodiments, each single unit dosage form comprising the compound of Formula A comprises: about 5 mg, about 10 mg, about 20 mg, about 25 mg, about 40 mg, about 80 mg, about 160 mg, about 200 mg, about 300 mg, about 400 mg, about 450 mg, about 500 mg or about 600 mg.

Each dosage amount administered to the patient can comprise 600 mg of the compound that may be sub-divided into two tablets comprising 300 mg of the compound.

Alternatively, each dosage amount can comprise 300 mg of the compound that may be one tablet comprising 300 mg of the compound.

In a specific embodiment, the patient is administered a daily dosage amount of 600 mg, which is administered as one dosage amount.

In another specific embodiment, the patient is administered a daily dosage amount of 1200 mg, which is administered as two dosage amounts, and in particular when the second dosage amount is administered between 2 and 6 hours of the first, preferably between about 3 and 6 hours of the first dosage amount.

In another specific embodiment, the patient is administered a daily dosage amount of 1800 mg, which is administered as three dosage amounts, and in particular when the second dosage amount is administered between 2 and 8 hours of the first (e.g. at about 2 hours, about 4 hours, about 6 hours, or about 8 hours), and the third dosage amount is administered between about 4 and 16 hours of the first dosage amount (e.g. at about 4 hours, about 6 hours, about 8 hours, about 12 hours, or about 16 hours).

The treatments of the invention involve oral administration. In any of the treatments of the invention, the compound of Formula A can be administered as an oral dosage form comprising the compound of Formula A and pharmaceutically acceptable excipients. The oral dosage form can be in the form of a tablet or a capsule. In one embodiment the oral dosage form is a tablet. In another embodiment, the oral dosage form is a capsule.

The treatments of the invention can comprise not administering more than three dosage amounts in a 24 hour period. Specifically, if each dosage amount comprises 600 mg of the compound, this means that the treatments of the inventions can comprise not administering more than 1800 mg of the compound in a 24 hour period. If each dosage amount comprising 600 mg of the compound is sub-divided into two dosage amounts (e.g. tablets) comprising 300 mg of the compound, the treatments of the invention can comprise administering not more than six dosage amounts each comprising 300 mg of the compound, in a 24 hour period, wherein each dosage amount can be a tablet.

As described above, the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and orodispersible tablet, or an orodispersible film.

The oral dosage forms used in the treatments of the invention can be selected from an orodispersible tablet, an orodispersible film, a syrup, a suspension, an emulsion, a solution and a chewable tablet or soft gels, which can be useful when administration is expected to take place in the absence of a drink (e.g. water).

The compound of Formula A (or a pharmaceutically acceptable salt and/or solvate thereof) can be administered as a dispersible tablet or an orodispersible tablet, which is especially suitable for patients between the ages of 2 and less than 18, more specifically between the ages of 2 and less than 12.

Dispersible tablets are intended to be uniformly dispersed or dissolved in water (or another drink) prior to administration. In the treatments of the invention, the compound of Formula A (or a pharmaceutically acceptable salt and/or solvate thereof) can be administered as a dispersible tablet (e.g. a dispersible mini-tablet), wherein the patient is between the ages of 2 and less than 18. More specifically, the compound of Formula A (or a pharmaceutically acceptable salt and/or solvate thereof) can be administered as a dispersible tablet, wherein the patient is between the ages of 2 and less than 12.

Orodispersible tablets (e.g. an orodispersible mini-tablet) are intended to be placed directly in the mouth where they rapidly disintegrate in saliva. Orodispersible tablets can therefore allow the patient or their guardian/carer to administer the treatment without having to find a glass of water to either aid swallowing or to reconstitute the dosage form for drinking. Orodispersible tablets (e.g. an orodispersible mini-tablet) are particularly preferred in the treatments of the invention, especially when a patient may struggle to swallow a normal tablet, e.g., wherein the bradykinin-mediated angioedema attack (e.g. a BK-AEnH attack, preferably an HAE attack) is a laryngeal attack; where the patients are elderly patients that struggle to swallow tablets (e.g. in patients older than 70 years old), or where the patients are young patients between the ages of 2 and less than 18, more specifically between the ages of 2 and less than 12. Orodispersible tablets (and indeed orodispersible films) are useful when administration is expected to take place in the absence of a drink (e.g. water).

Thus, in the treatments of the invention, the compound of Formula A (or a pharmaceutically acceptable salt and/or solvate thereof) can be administered as an orodispersible tablet (e.g. an orodispersible mini-tablet). More specifically, the compound of Formula A (or a pharmaceutically acceptable salt and/or solvate thereof) can be administered as an orodispersible tablet (e.g. an orodispersible mini-tablet), wherein the patient is between the ages of 2 and less than 18, more specifically between the ages of 2 and less than 12. Alternatively, or additionally, the compound of Formula A (or a pharmaceutically acceptable salt and/or solvate thereof) can be administered as an orodispersible tablet (e.g. an orodispersible mini-tablet), wherein the patient is suffering from a laryngeal attack.

Alternatively, the compound of Formula A (or a pharmaceutically acceptable salt and/or solvate thereof) can be administered as an orodispersible tablet, wherein the patient is an elderly patient that struggles to swallow tablets (e.g. in patients older than 70 years old). Alternatively, or additionally, the compound of Formula A (or a pharmaceutically acceptable salt and/or solvate thereof) can be administered as an orodispersible tablet, wherein the patient is suffering from a laryngeal attack.

When the compound of Formula A (or a pharmaceutically acceptable salt and/or solvate thereof) is administered as an orodispersible table or a dispersible tablet (preferably an orodispersible tablet) the disintegration specification can be >80% in less than about 10 minutes. As used here, the disintegration specification is obtained in accordance with the standard procedure in USP <701> and Ph Eur 2.9.1 (measured in deionised water). More specifically, the disintegration specification can be >80% in less than about 5 minutes. More specifically, the disintegration specification can be >80% in less than about 2 minutes. More specifically, and preferably, the disintegration specification can be >80% in less than about 1 minute. The disintegration specification can be >80% in less than about 45 seconds. The disintegration specification can be >80% in less than about 30 seconds.

Additionally, or alternatively, when the compound of Formula A (or a pharmaceutically acceptable salt and/or solvate thereof) is administered as an orodispersible tablet or a dispersible tablet (preferably an orodispersible tablet), the dissolution specification can be ≥80% in less than about 45 minutes. As used herein, the dissolution specification can be determined by the method outlined below. More specifically, the dissolution specification can be ≥80% in less than about 30 minutes. More specifically, the dissolution specification can be ≥≥80% in less than about 20 minutes. More specifically, the dissolution specification can be ≥80% in less than about 10 minutes. More specifically, the dissolution specification can be ≥80% in less than about 5 minutes.

Specifically, when the compound of Formula A (or a pharmaceutically acceptable salt and/or solvate thereof) is administered as an orodispersible tablet or a dispersible tablet (preferably an orodispersible tablet), the disintegration specification can be >80% in less than about 10 minutes, and the dissolution specification can be ≥80% in less than about 45 minutes. The disintegration specification can be >80% in less than about 10 minutes, and the dissolution specification can be >80% in less than about 30 minutes. The disintegration specification can be >80% in less than about 10 minutes, and the dissolution specification can be ≥80% in less than about 20 minutes. The disintegration specification can be >80% in less than about 5 minutes, and the dissolution specification can be ≥80% in less than about 20 minutes. The disintegration specification can be >80% in less than about 5 minutes, and the dissolution specification can be ≥80% in less than about 10 minutes. The disintegration specification can be >80% in less than about 2 minutes, and the dissolution specification can be ≥80% in less than about 10 minutes. The disintegration specification can be >80% in less than about 2 minutes, and the dissolution specification can be ≥80% in less than about 5 minutes. The disintegration specification can be >80% in less than about 1 minute, and the dissolution specification can be ≥80% in less than about 5 minutes.

Chewable tablets or soft gels are intended to be chewed before being swallowed. The compound of Formula A (or a pharmaceutically acceptable salt and/or solvate thereof) can be administered as a chewable tablet or soft gel. The compound of Formula A (or a pharmaceutically acceptable salt and/or solvate thereof) can be administered as a chewable tablet. The compound of Formula A (or a pharmaceutically acceptable salt and/or solvate thereof) can be administered as a chewable soft gel. When administered as a chewable tablet or soft gel, the dissolution specification can be ≥80% in less than about 45 minutes. The dissolution specification can be determined by the method outlined below. More specifically, the dissolution specification can be ≥80% in less than about 30 minutes. More specifically, the dissolution specification can be ≥80% in less than about 20 minutes. More specifically, the dissolution specification can be ≥80% in less than about 10 minutes. More specifically, the dissolution specification can be ≥80% in less than about 5 minutes.

The compound of Formula A (or a pharmaceutically acceptable salt and/or solvate thereof) can be administered as a mini-tablet. A mini-tablet can have a diameter in the range of 2 to 3 mm.

The compound of Formula A (or a pharmaceutically acceptable salt and/or solvate thereof) can be administered as a powder. When administered as a powder, the dissolution specification can be ≥80% in less than about 45 minutes. The dissolution specification can be determined by the method outlined below. More specifically, the dissolution specification can be ≥80% in less than about 30 minutes. More specifically, the dissolution specification can be ≥80% in less than about 20 minutes. More specifically, the dissolution specification can be ≥80% in less than about 10 minutes. More specifically, the dissolution specification can be ≥80% in less than about 5 minutes. Additionally, or alternatively, the disintegration specification can be >80% in less than about 10 minutes. More specifically, the disintegration specification can be >80% in less than about 5 minutes. More specifically, the disintegration specification can be >80% in less than about 2 minutes. More specifically, and preferably, the disintegration specification can be >80% in less than about 1 minute.

The compound of Formula A (or a pharmaceutically acceptable salt and/or solvate thereof) can be administered as granules. When administered as granules, the dissolution specification can be ≥80% in less than about 45 minutes. The dissolution specification can be determined by the method outlined below. More specifically, the dissolution specification can be ≥80% in less than about 30 minutes. More specifically, the dissolution specification can be ≥80% in less than about 20 minutes. More specifically, the dissolution specification can be ≥80% in less than about 10 minutes. More specifically, the dissolution specification can be ≥80% in less than about 5 minutes. Additionally, or alternatively, the disintegration specification can be ≥80% in less than about 10 minutes. More specifically, the disintegration specification can be >80% in less than about 5 minutes. More specifically, the disintegration specification can be >80% in less than about 2 minutes. More specifically, and preferably, the disintegration specification can be >80% in less than about 1 minute.

The compound of Formula A (or a pharmaceutically acceptable salt and/or solvate thereof) can be administered as a solution.

The compound of Formula A (or a pharmaceutically acceptable salt and/or solvate thereof) can be administered as an emulsion.

The compound of Formula A (or a pharmaceutically acceptable salt and/or solvate thereof) can be administered as a suspension. When administered as a suspension, the dissolution specification can be ≥80% in less than about 45 minutes. The dissolution specification can be determined by the method outlined below. More specifically, the dissolution specification can be ≥80% in less than about 30 minutes. More specifically, the dissolution specification can be ≥80% in less than about 20 minutes. More specifically, the dissolution specification can be ≥80% in less than about 10 minutes. More specifically, the dissolution specification can be ≥80% in less than about 5 minutes.

The compound of Formula A (or a pharmaceutically acceptable salt and/or solvate thereof) can be administered as a syrup.

The oral dosage forms can comprise one or more of a diluent, a binder, a disintegrant, and a lubricant. In particular, the compound of Formula A (or a pharmaceutically acceptable salt and/or solvate thereof) can be administered as an orodispersible tablet, a dispersible tablet, or a chewable tablet or soft gel, and it can comprise one or more of a diluent, a binder, a disintegrant, and a lubricant.

The compound of Formula A can be present in the dosage form between about 10 and about 70 wt % of the total dosage form. The compound of Formula A can be present in the dosage form between about 20 and about 60 wt % of the total dosage form. The compound of Formula A can be present in the dosage form between about 30 and about 70 wt % of the total dosage form. The compound of Formula A can be present in the dosage form between about 20 and about 50 wt % of the total dosage form. The compound of Formula A can be present in the dosage form between about 20 and about 40 wt % of the total dosage form. The compound of Formula A can be present in the dosage form between about 30 and about 40 wt % of the total dosage form. The compound of Formula A can be present in the dosage form between about 40 and about 70 wt % of the total dosage form. The compound of Formula A can be present in the dosage form between about 50 and about 70 wt % of the total dosage form. The compound of Formula A can be present in the dosage form between about 50 and about 60 wt % of the total dosage form.

The diluent can be selected from lactose (e.g. lactose monohydrate, spray dried lactose, anhydrous lactose), sorbitol, mannitol, sucrose, starch and pregelatinized starch, cellulose (e.g. powdered cellulose, microcrystalline cellulose e.g. Avicel PH101, Avicel PH102), calcium phosphates (e.g. anhydrous dibasic calcium phosphate, dibasic calcium phosphate, tribasic calcium phosphate), tricalcium citrate, and mixtures thereof.

The diluent can be present at between about 20 and about 80 wt % of the total dosage form. The diluent can be present at between about 30 and about 80 wt % of the total dosage form. The diluent can be present at between about 40 and about 80 wt % of the total dosage form. The diluent can be present at between about 40 and about 70 wt % of the total dosage form. The diluent can be present at between about 50 and about 70 wt % of the total dosage form. The diluent can be present at between about 60 and about 80 wt % of the total dosage form.

The disintegrant is selected from polysaccharides (such as starch, starch 1500, alginic acid and salts, guar gum, soy polysaccharides), various grades of microcrystalline cellulose (e.g. Avicel PH101, Avicel PH102), cellulose derivatives (e.g. methyl cellulose, carboxymethyl cellulose and salts, hydroxypropyl methylcellulose), crosslinked cellulose (e.g. Croscarmellose®, Ac-Di-Sol®, Nymce ZSX®, Primellose®, Solutab®, Vivasol®), synthetic polymers such as cross linked polyvinylpyrrolidone (e.g. Crospovidone, Crospovidon M®, Kollidon®, Polyplasdone®), inorganic compounds (such as calcium silicate), sodium starch glycolate, and mixtures thereof. More specifically, the disintegrant can be starch USP, starch 1500, microcrystalline cellulose (or various grades thereof e.g. Avicel PH101, Avicel PH102), cellulose derivatives (e.g. methyl cellulose, carboxymethyl cellulose and salts, hydroxypropyl methylcellulose), guar gum, alginic acid and salts, sodium starch gylcolate, crosslinked cellulose (e.g. croscarmellose sodium such as those traded under the names Croscarmellose®, Ac-Di-Sol®, Nymce ZSX®, Primellose®, Solutab®, Vivasol®), cross linked polyvinylpyrrolidone (e.g. Crospovidone, Crospovidon M®, Kollidon®, Polyplasdone®), soy polysaccharides, calcium silicate, pearlitol Flash (co-processed Mannitol/Maize starch) and mixtures thereof.

The disintegrant can be present at between about 1 and about 50 wt % of the total dosage form. The disintegrant can be present at between about 2 and about 45 wt % of the total dosage form. The disintegrant can be present at between about 3 and about 30 wt % of the total dosage form. The disintegrant can be present at between about 3 and about 20 wt % of the total dosage form. The disintegrant can be present at between about 3 and about 15 wt % of the total dosage form. The disintegrant can be present at between about 5 and about 15 wt % of the total dosage form.

The binder can be selected from saccharides (such as glucose, sucrose, fructose, dextrose, lactose), sugar alcohols (such as mannitol, xylitol, maltitol, erythritol, sorbitol), polysaccharides (such as crystalline cellulose, microcrystalline cellulose, powdered cellulose, methyl cellulose, carboxymethyl cellulose and salts, hydroxypropylmethyl cellulose), corn starch, starch 1500, acacia, other natural polymers (such as gelatin), synthetic polymers (such as polyvinyl pyrrolidone, polyvinyl polypyrolidone (crospovidone)), polyethylene gylcol, crosslinked acrylic acid and acrylic acid copolymers (e.g. Carbopol®), inorganic compounds (such as calcium carbonate), and mixtures thereof. More specifically, the binder can be selected from glucose, lactose, corn starch, starch 1500, gelatin, acacia, methyl cellulose, carboxymethyl cellulose and salts, hydroxypropylmethyl cellulose, polyvinyl pyrrolidone, and mixtures thereof.

The binder can be present at between about 20 and about 50 wt % of the total dosage form. The binder can be present at between about 30 and about 50 wt % of the total dosage form. The binder can be present at between about 35 and about 50 wt % of the total dosage form.

The lubricant can be selected from magnesium stearate, calcium stearate, stearic acid, hydrogenated vegetable oil, mineral oil, glyceryl behenate, glyceryl palmitostearate, polyethylene glycol, polyoxyethylene stearates, lauryl sulphate, talc, paraffin, sodium stearyl fumarate, and mixtures thereof.

The lubricant can be present at between about 0.1 and 15 wt % of the total dosage form. The lubricant can be present at between about 0.1 and 10 wt % of the total dosage form. The lubricant can be present at between about 0.5 and 10 wt % of the total dosage form. The lubricant can be present at between about 1 and 10 wt % of the total dosage form. The lubricant can be present at between about 2 and 8 wt % of the total dosage form. The lubricant can be present at between about 3 and 8 wt % of the total dosage form. The lubricant can be present at between about 4 and 6 wt % of the total dosage form.

When the compound of Formula A (or a pharmaceutically acceptable salt and/or solvate thereof) is administered as an orodispersible tablet, the orodispersible tablet can comprise one or more of microcrystalline cellulose as a diluent; mannitol as a binder; a lubricant selected from magnesium stearate, sodium stearyl fumarate and stearic acid; and a disintegrant selected from polyvinyl polypyrolidone (crospovidone), sodium starch glycolate and crosslinked cellulose (e.g. Croscarmellose®, Ac-Di-Sol®, Nymce ZSX®, Primellose®, Solutab®, Vivasol®, particularly Croscarmellose®).

More specifically, when the compound of Formula A (or a pharmaceutically acceptable salt and/or solvate thereof) is administered as an orodispersible tablet, the orodispersible tablet can comprise two or more of microcrystalline cellulose as a diluent; mannitol as a binder; a lubricant selected from magnesium stearate, sodium stearyl fumarate and stearic acid; and a disintegrant selected from polyvinyl polypyrolidone (crospovidone), sodium starch glycolate and crosslinked cellulose (e.g. Croscarmellose®, Ac-Di-Sol®, Nymce ZSX®, Primellose®, Solutab®, Vivasol®, particularly Croscarmellose®).

More specifically, when the compound of Formula A (or a pharmaceutically acceptable salt and/or solvate thereof) is administered as an orodispersible tablet, the orodispersible tablet can comprise three or more of microcrystalline cellulose as a diluent; mannitol as a binder; a lubricant selected from magnesium stearate, sodium stearyl fumarate and stearic acid; and a disintegrant selected from polyvinyl polypyrolidone (crospovidone), sodium starch glycolate and crosslinked cellulose (e.g. Croscarmellose®, Ac-Di-Sol®, Nymce ZSX®, Primellose®, Solutab®, Vivasol®, particularly Croscarmellose®).

When the compound of Formula A (or a pharmaceutically acceptable salt and/or solvate thereof) is administered as an orodispersible tablet, the orodispersible tablet can comprise microcrystalline cellulose as a diluent; mannitol as a binder; a lubricant selected from magnesium stearate, sodium stearyl fumarate and stearic acid; and a disintegrant selected from polyvinyl polypyrolidone (crospovidone), sodium starch glycolate and crosslinked cellulose (e.g. Croscarmellose®, Ac-Di-Sol®, Nymce ZSX®, Primellose®, Solutab®, Vivasol®, particularly Croscarmellose®).

When the compound of Formula A (or a pharmaceutically acceptable salt and/or solvate thereof) is administered as an orodispersible tablet, the orodispersible tablet can comprise one or more of microcrystalline cellulose, sodium starch glycolate, croscarmellose sodium, camphor, sodium saccharin, and magnesium stearate.

When the compound of Formula A (or a pharmaceutically acceptable salt and/or solvate thereof) is administered as an orodispersible tablet, the orodispersible tablet can comprise one or more of pearlitol Flash (co-processed Mannitol/Maize starch), sucralose, and sodium stearyl fumarate.

When the compound of Formula A (or a pharmaceutically acceptable salt and/or solvate thereof) is administered as a chewable tablet, a chewable tablet can comprise one or more of mannitol, microcrystalline cellulose, hydroxypropyl cellulose, croscarmellose sodium, strawberry flavour, sucralose, and magnesium stearate.

When the compound of Formula A (or a pharmaceutically acceptable salt and/or solvate thereof) is administered as a mini-tablet, a mini-tablet can comprise one or more of microcrystalline cellulose, croscarmellose sodium, polyvinylpyrrolidone (povidone), and magnesium stearate. The mini-tablet can be coated with PVA/Titanium oxide, PEG, talc based coating.

When the compound of Formula A (or a pharmaceutically acceptable salt and/or solvate thereof) is administered as a coated granules, a coated granules can comprise one or more of microcrystalline cellulose, croscarmellose sodium, polyvinylpyrrolidone (povidone), and magnesium stearate. The granules can be coated with PVA/Titanium oxide, PEG, talc based coating.

When the compound of Formula A (or a pharmaceutically acceptable salt and/or solvate thereof) is administered as an oral suspension, an oral suspension can comprise one or more of sorbitol solution (70%), glycerol, dispersible cellulose, sodium carboxymethylcellulose, polysorbate 80, methyl hydroxybenzoate, sodium benzoate, sodium saccharin, ethanol, and vanilla flavour.

Any of the oral dosage forms described herein can also comprise a sweetener such as a sweetener selected from sucralose, xylitol, fructose, sucrose, and sodium saccharin (and mixtures thereof).

Any of the oral dosage forms described herein can also comprise a flavouring such as a flavouring selected from strawberry flavouring, a raspberry flavouring, a cherry flavouring, and a vanilla flavouring (and mixtures thereof).

Any of the oral dosage forms described herein can be coated (when it is appropriate to do so). The coating can comprise one or more of PVA, titanium oxide, PEG, or a talc based coating.

Dissolution Testing

The dissolution testing can be conducted in accordance with the following protocol-USP <711> and Ph Eur 2.9.3.

Equipment: Dissolution bath with HPLC-UV

Preparation of Reagents:
Reagents
   Water, Analytical grade
   Acetonitrile, HPLC grade
   Formic acid, HPLC grade
   Concentrate (37%) Hydrochloric acid (AR grade)

Dissolution Media (10 L):
0.1 M HCl Buffer solution
Transfer 86 mL of Concentrate (37%) Hydrochloric acid (AR grade) and add to 9.89 L of Analytical Grade
Water, mix until dissolved

Sample Solvent
0.1 M HCl Buffer solution

Mobile Phase A (2 L)
0.05% formic acid in Analytical Grade Water
To prepare 2 L add 1 mL formic acid to 2 L Analytical Grade Water and mix well. Sonicate for 10 minutes to degas

### Mobile Phase B (2 L)

0.05% formic acid in Acetonitrile, HPLC grade
To prepare 2 L add 1 mL formic acid to 2 L in Acetonitrile, HPLC grade and mix well. Sonicate for 10 minutes to degas

Needle Wash (1 L)
90% Acetonitrile, HPLC grade in Analytical Grade Water
To prepare 1 L add 900 mL Acetonitrile, HPLC grade to 100 mL of Analytical Grade Water mix well. Sonicate for 10 minutes to degas

Preparation of Analytical Solutions
Preparation of Standards (100 μg/mL)
Standards can be prepared in duplicate in amber glass and are stable for 5 days at 2-8° C. in amber glass.

Preparation of Standards (100 μg/mL)
Accurately weigh approximately 20 mg+/−1 mg of Compound of Formula A (free base) into a 200 mL volumetric flask.
Add approximately 100 mL of Sample Solvent and sonicate/swirl to dissolve.
Dilute to volume with Sample Solvent.

This is the Working Standard Solution (100 μg/mL).

Preparation of Samples (100 μg/mL)—Table A shows the dissolution parameters.

TABLE A
dissolution parameters

| Parameter | Requirement |
|---|---|
| Apparatus | USP II (Paddles) |
| Paddle Speed | 50 rpm ± 4 rpm |
| Dissolution Media | 0.1M HCl |
| Dissolution Media | 1000 mL degassed in amber vessels |
| Volume | |
| Dissolution Media | 37° C. ± 0.5° C. |
| Temperature | |
| Sample Volume | 5.0 mL |
| Cannula Filter | 4 μm or 10 μm cannula filter |
| Sample Filter | 0.2 μm PVDF or 0.22 μm PTFE syringe filters |
| Sampling points | (50 rpm) 5, 10, 20, 30, and 45 minutes |
| | (200 rpm) 60 minutes |
| Dissolution time | 60 minutes |
| Sample Analysis | HPLC, UV @ 225 nm |

Procedure
In each of the vessels, place 1000 mL degassed dissolution media and allow to equilibrate to 37° C.±0.5° C.
Set up bath and fit cannula filters.
Record the temperature of each vessel prior to starting and at the end of the test.
Weigh and record an appropriate number of dosage forms and drop them into the relevant vessel, avoiding the paddle.
Commence the dissolution test using the specified parameters.
At the specified sampling point, manually, prime each canula and syringe two times immediately prior to withdrawing sample.
Withdraw 5 mL of sample from the vessel filter through a 0.2 am PVDF or 0.22 am PTFE membrane filter, discarding the first 1 mL of filtrate.

Analysis

Chromatographic Details—Column Waters CSH C18, 2.1×75 mm, 1.7 am 0.2 am in-line column filter, with the details in Table B.

TABLE B

| | |
|---|---|
| Column temperature | 60° C. |
| Flow rate | 0.7 mL/min |
| Detector | 225 nm with VWD |
| Injection volume | 1.5 μL |
| Sampling rate | 10 points per second |
| Run time | 2.5 minutes |
| Mobile phase | Mobile Phase A: 0.05% Formic Acid in H₂O |
| composition | Mobile Phase B: 0.05% Formic Acid in ACN |
| | (50 rpm) 5, 10, 20, 30, and 45 minutes |
| | (200 rpm) 60 minutes |

TABLE C
Gradient

| Time | % A | % B |
|---|---|---|
| 0.00 | 90 | 10 |
| 0.50 | 50 | 50 |
| 1.31 | 50 | 50 |
| 1.32 | 90 | 10 |
| 2.50 | 90 | 10 |

Multiple injections from a single vial can be taken, for example Standard A. Not more than 12 sample injections are to be performed between standard injections. To aid analysis, the sequence may be run as a series of smaller sample sets. When this approach is applied the system suitability criteria must be applied across all the sample sets. Consecutive sample sets may only be run if the instrument conditions have not been altered, for example the introduction of fresh mobile phase will require a repeat of the system suitability sequence.

Further Specifics of the Treatments of the Invention

As shown herein, the compound of Formula A has a rapid onset of action. Specifically, the compound of Formula A is a potent inhibitor of plasma kallikrein activity and is highly effective at interrupting the contact activation system's positive feedback loop between plasma kallikrein, prekallikrein, Factor XII (FXII), and Factor XIIa (FXIIa). The pharmacokinetic and pharmacodynamic data provided herein demonstrate that these effects are shown quickly after oral administration of the compound of Formula A. Accordingly, the treatments of the invention are fast acting and are thus particularly suited to treating bradykinin-mediated angioedema (e.g. BK-AEnH, preferably HAE) on-demand.

As discussed above, the treatments of the invention are particularly advantageous when the concentration of the compound of Formula A is at least 500 ng/mL in plasma. A plasma concentration of at least 500 ng/mL can be observed following administration of a dosage amount of at least about 60 mg (more specifically, at least about 70 mg or about 80 mg) of the compound of Formula A.

The treatments according to the invention provide rapid protection from HK (high molecular weight kininogen) cleavage that are particularly suited to prophylactically reducing the chances of a bradykinin-mediated angioedema attack (e.g. a BK-AEnH attack, preferably an HAE attack) and/or to shorten the severity (or even halt) an ongoing bradykinin-mediated angioedema attack (e.g. a BK-AEnH attack, preferably an HAE attack). As described here, the treatments according to the invention also have a prolonged pharmacodynamic effect. The pharmacodynamic effects of the compound of Formula A that are related to treating bradykinin-mediated angioedema (e.g. BK-AEnH, preferably HAE) include providing protection from HK cleavage, which as discussed above, can cause a bradykinin-mediated angioedema attack (e.g. a BK-AEnH attack, preferably an HAE attack). For example, the compound of Formula A can provide protection from HK cleavage by at least (i) inhibiting plasma kallikrein, (ii) reducing cleavage of plasma prekallikrein, and/or (iii) reducing the generation of Factor XIIa from Factor XII.

In some embodiments, the treatments according to the invention can provide protection from HK (high molecular weight kininogen) cleavage within one hour post-dosage amount, and in particular when the dosage amount of the compound of Formula A is at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg). In some embodiments, the treatments according to the invention can provide protection from HK (high molecular weight kininogen) cleavage within 45 minutes post-dosage amount, or within 30 minutes post-dosage amount. In these embodiments, protection from HK (high molecular weight kininogen) cleavage is determined by comparing HK levels in untreated plasma with HK levels in treated plasma i.e. plasma from subjects that have received a dosage amount of the compound of Formula A, and then activating the plasma with dextran sulfate to activate the contact system to induce HK cleavage. If the HK level in the treated plasma is above the HK level in the untreated plasma, then the HK has been protected from HK cleavage in the activated plasma.

In some embodiments of the invention, the treatment can inhibit at least 80% of plasma kallikrein activity within 30 minutes post-dosage amount, and in particular when the dosage amount of the compound of Formula A is at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg). In some embodiments of the invention, the treatment can inhibit at least 90% of plasma kallikrein activity within 30 minutes post-dosage amount, and in particular when the dosage amount of the compound of Formula A is at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg). In some embodiments of the invention, the treatment can inhibit at least 95% of plasma kallikrein activity within 30 minutes post-dosage amount, and in particular when the dosage amount of the compound of Formula A (or a pharmaceutically acceptable salt and/or solvate thereof is at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg). In embodiments where the inhibition of plasma kallikrein activity is mentioned, inhibition of plasma kallikrein activity is determined by time-dependent hydrolysis of fluorogenic substrate (e.g. (H-D-Pro-Phe-Arg-AFC; Peptide Protein Research) according to procedures known in the art. In these embodiments, inhibition of plasma kallikrein activity is determined in plasma obtained from subjects that have taken a dosage amount of the compound of Formula A which has subsequently been activated with dextran sulfate to emulate a HAE situation.

In some embodiments of the invention, a therapeutically effective concentration of the compound of Formula A can be achieved within 20 minutes post-dosage amount.

In some embodiments of the invention, the Tₘₐₓ of the compound of Formula A can be between 30 minutes and 3 hours post-dosage amount, preferably between 30 minutes and 2 hours post-dosage amount.

In some embodiments of the invention, the treatment can inhibit at least 90% of plasma kallikrein activity for at least the period of time between 45 minutes and 2 hours post-dosage amount, and in particular when the dosage amount of the compound of Formula A is between 100 mg and 200 mg (preferably 160 mg). In some embodiments, the treatment can inhibit at least 90% of plasma kallikrein activity for at least the period of time between 20 minutes and 4 hours post-dosage amount, and in particular when the dosage amount of the compound of Formula A is between 100 mg and 200 mg (preferably 160 mg). In some embodiments, the treatment can inhibit at least 90% of plasma kallikrein activity for at least the period of time between 30 minutes and 10 hours post-dosage amount, and in particular when the dosage amount of the compound of Formula A is between 300 mg and 800 mg (preferably 600 mg). In some embodiments, the treatment can inhibit at least 95% of plasma kallikrein activity for at least the period of time between 20 minutes and 6 hours post-dosage amount, and in particular when the dosage amount of the compound of Formula A is between 300 mg and 800 mg (preferably 600 mg). In some embodiments, the treatment can inhibit at least 99% of plasma kallikrein activity for at least the period of time between 20 minutes and 6 hours post-dosage amount, and in particular when the dosage amount of the compound of Formula A is between 300 mg and 800 mg (preferably 600 mg). Again, in these embodiments, inhibition of plasma kallikrein activity is determined in plasma obtained from subjects that have taken a dosage amount of the compound of Formula A which has subsequently been activated with dextran sulfate to emulate a HAE situation.

In some embodiments, the pharmacodynamic effects of the compound of Formula A that are related to treating bradykinin-mediated angioedema (e.g. BK-AEnH, preferably HAE) can be maintained for at least 12 hours post-dosage amount, and in particular when the dosage amount of the compound of Formula A is between 300 mg and 800 mg (preferably 600 mg). In some embodiments, the treatment can inhibit at least 50% of plasma kallikrein activity for at least 10 hours post-dosage amount, and in particular when the dosage amount of the compound of Formula A is between 100 mg and 200 mg (preferably 160 mg). In these embodiments, pharmacodynamic effects means at least (i) inhibition of plasma kallikrein, (ii) protection from HK cleavage/reduction of HK cleavage, (iii) protection from (or a reduction of) Factor XII cleavage to generate Factor XIIa, and/or (iv) protection from (or a reduction of) plasma prekallikrein cleavage to generate plasma kallikrein. Treatments according to the invention are therefore suitable candidates for being advantageously efficacious treatments of bradykinin-mediated angioedema attacks (e.g. BK-AEnH attacks, preferably HAE attacks) because they are fast-acting and potent (e.g. inhibitory) over a sufficiently long period of time.

As discussed above, in any of the treatments of the invention, the compound of Formula A can inhibit plasma kallikrein.

In any of the treatments of the invention, particularly following a dosage amount of the compound of Formula A of at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg), the compound of Formula A can inhibit Factor XII cleavage to generate Factor XIIa. In any of the treatments of the invention, particularly following a dosage amount of the compound of Formula A of at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg), the compound of Formula A can inhibit plasma prekallikrein cleavage into plasma kallikrein. In any of the treatments of the invention, particularly following a dosage amount of the compound of Formula A of at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg), the compound of Formula A can result in the inhibition (e.g. blockage) of contact system activation for up to 6 hours post-dosage amount. In some embodiments, where a dosage amount of at least about 60 mg (more specifically, at least about 70 mg or about 80 mg such as about 80 mg to about 900 mg, about 100 mg to about 800 mg, about 200 mg to about 700 mg, about 300 mg to about 600 mg, or about 400 mg to about 600 mg, specifically 600 mg) is administered, the contact system activation can be inhibited (e.g. blocked) for at least 6 hours e.g. for between 6 hours and 12 or 18 hours post-dosage amount.

### FIGURES

In the Figures, the term “Compound” means the compound of Formula A.

FIG. 1: X-ray powder diffraction pattern of the compound of Formula A as generated in Example 1.

FIG. 2A: Assay results showing plasma kallikrein inhibition activity of the compound of Formula A and a C1 inhibitor C1-INH in dextran sulfate (DXS)-activated diluted plasma.

FIG. 2B: Assay results showing plasma kallikrein inhibition activity of the compound of Formula A and a C1 inhibitor (C1-INH) in DXS-activated undiluted plasma.

FIG. 3A: Assay results comparing the plasma kallikrein inhibition activity of the compound of Formula A and C1-INH in DXS-activated diluted plasma.

FIG. 3B: Assay results comparing inhibition activity of the compound of Formula A and C1-INH following addition to pre-activated undiluted human plasma. Data are expressed as total fluorescence over time (Fluorescence Units) mean±SEM of n=3 experiments.

FIG. 4A: Assay (bioanalytical) results showing plasma concentrations of the compound of Formula A between 0 and 24 hours post-dose, in fasted subjects from eight (8) single ascending dose cohorts.

FIG. 4B: Table of Cₘₐₓ values determined from the assay (bioanalytical) results shown in FIG. 4A.

FIG. 5A: Assay results showing plasma kallikrein activity in DXS-activated undiluted plasma for cohorts 6 to 8 (160 mg, 300 mg, and 600 mg).

FIG. 5B: Assay results showing the mean plasma kallikrein activity and mean plasma concentration of the compound of Formula A in undiluted plasma in subjects from cohort 8 (600 mg dose).

FIG. 6A: Assay results showing the mean fluorescent kinetic measurements indicating a lag time in catalytic activity during contact system activation in DXS-activated undiluted plasma of a subject who has received a 600 mg dose of the compound of Formula A.

FIG. 6B: An enlargement of FIG. 6A between 0 and 5 mins following catalytic activation.

FIG. 7: Assay results showing mean percent HK protection at selected time points post-dosage in DXS-activated undiluted plasma for cohorts 6 to 8 (160 mg, 300 mg, and 600 mg), and a representation WES gel image of the immunoblot data.

FIG. 8: Assay results showing the effect of the compound of Formula A on DXS-activated HK cleavage at selected time points post-dosage in cohort 8 (600 mg), and a representation WES gel image of the immunoblot data.

FIG. 9: Assay results showing the effect of the compound of Formula A on DXS-activated plasma prekallikrein (PPK) cleavage, at selected time points post-dosage in cohort 8 (600 mg), and a representation WES gel image of the immunoblot data.

FIG. 10: Assay results showing the effect of the compound of Formula A on DXS-activated generation of FXIIa, at selected time points post-dosage in cohort 8 (600 mg), and a representation WES gel image of the immunoblot data.

FIG. 11: Assay (bioanalytical) results showing the effect of the plasma concentration of the compound of Formula A at various stages post-dose in cohort 8 (600 mg) at time points selected for HK, FXIIa, PPK analysis.

FIG. 12: Assay results showing no significant food effect on the plasma kallikrein inhibitory activity of the compound of Formula A in DXS-activated undiluted plasma.

FIGS. 13A and 13B: Assay results showing a time course of dextran sulfate-activated cleavage of HK in HAE whole undiluted plasma determined using western blotting, and a representative blot image.

FIGS. 14A and 14B: Assay results showing the dose response of the compound of Formula A on full length HK levels in dextran sulfate-activated healthy control plasma and HAE plasma, and representative WES system gel images.

FIG. 15a: Preliminary pharmacokinetic data from the phase 2 study.

FIG. 15b: PK profile of HAE patients from part 1 of the phase 2 study, overlaid with PK data from healthy volunteers.

FIG. 15c: Graph showing cumulative % of patients that used rescue medication within 12 hours of being administered the compound.

FIG. 15d: Graph showing cumulative % of patients with symptom relief (as assessed using the 7TQ method) within 12 hours of being administered the compound.

FIG. 15e: Graph showing comparison of time to symptom relief data for the compound with known clinical data for approved injectable product Ruconest®.

FIG. 15f: Graph showing cumulative % of patients with symptom relief (as assessed using the composite VAS score) within 12 hours of being administered the compound.

FIG. 15g: Graph showing mean composite VAS score within 12 hours of being administered the compound.

FIG. 15h: Graph showing comparison of mean composite VAS score for patients treated with the compound with known clinical data for approved injectable product Firazyr® (active ingredient, icatibant).

FIG. 15i: Graph showing the % of HAE attacks that did not worsen (as assessed using the 5LS method) within 12 hours of being administered the compound.

FIG. 15i: Graph showing % of HAE attacks that improved in severity to “none” on the 5LS (also called the PGI-S) within 24 hours of being administered the compound compared with placebo.

FIG. 16A: Mean plasma concentrations over time of 4 cohorts in the phase 1 multiple dose study.

FIG. 16B: Mean plasma concentrations over time (semi-logarithmic scale) of 4 cohorts in the phase 1 multiple dose study.

Embodiments provided herein may be more fully understood by reference to the following examples. These examples are meant to be illustrative of treatments provided herein, but are not in any way limiting. Indeed, the scope of the invention is defined by the claims.

While examples of certain particular embodiments are provided herein, it will be apparent to those skilled in the art that various changes and modifications may be made. Such modifications are also intended to fall within the scope of the appended claims.

General Experimental Details

In the following examples, the following abbreviations and definitions are used:

| | |
|---|---|
| Aq | Aqueous solution |
| DCM | Dichloromethane |
| DMF | N,N-Dimethylformamide |
| DMSO | Dimethyl sulfoxide |
| DXS | Dextran sulfate |
| EtOAc | Ethyl Acetate |
| FXII | Factor XII |
| FXIIa | Factor XIIa |
| HK | High molecular weight kininogen |
| Hrs | Hours |
| HOBt | Hydroxybenzotriazole |
| IPA | 2-Propanol/Propan-2-ol/Iso-propanol |
| LCMS | Liquid chromatography mass spectrometry |
| Me | Methyl |
| MeCN | Acetonitrile |
| MeOH | Methanol |
| Min | Minutes |
| MS | Mass spectrum |
| NMR | Nuclear magnetic resonance spectrum—NMR |
| | spectra were recorded at a frequency of |
| | 400 MHz unless otherwise indicated |
| Pet. Ether | Petroleum ether fraction boiling at 60-80° C. |
| Ph | Phenyl |
| PKa | Plasma kallikrein |
| PPK | Plasma prekallikrein |
| SWFI | Sterile water for injection |
| Rt | room temperature |
| THF | Tetrahydrofuran |
| XRPD | X-ray powder diffraction |

All reactions were carried out under an atmosphere of nitrogen unless specified otherwise.

¹H NMR spectra were recorded on a Bruker (400 MHz) or on a JEOL (400 MHz) spectrometer with reference to deuterium solvent and at rt.

Molecular ions were obtained using LCMS which was carried out using a Chromolith Speedrod RP-18e column, 50×4.6 mm, with a linear gradient 10% to 90% 0.1% HCO₂H/MeCN into 0.1% HCO₂H/H₂O over 13 min, flow rate 1.5 mL/min, or using Agilent, X-Select, acidic, 5-95% MeCN/water over 4 min. Data was collected using a Thermofinnigan Surveyor MSQ mass spectrometer with electospray ionisation in conjunction with a Thermofinnigan Surveyor LC system.

Alternatively, molecular ions were obtained using LCMS which was carried out using an Agilent Poroshell 120 EC-C18 (2.7 μm, 3.0×50 mm) column with 0.1% v/v Formic acid in water [eluent A]; MeCN [eluent B];

Flow rate 0.8 mL/min and 1.5 minutes equilibration time between samples, gradient shown below. Mass detection was afforded with API 2000 mass spectrometer (electrospray).

Gradient:

| Time (min) | Eluent A (%) | Eluent B (%) |
|---|---|---|
| 0.00 | 95 | 5 |
| 0.20 | 95 | 5 |
| 2.00 | 5 | 95 |
| 3.00 | 5 | 95 |
| 3.25 | 95 | 5 |
| 3.50 | 95 | 5 |

Where products were purified by flash chromatography, ‘silica’ refers to silica gel for chromatography, 0.035 to 0.070 mm (220 to 440 mesh) (e.g. Merck silica gel 60), and an applied pressure of nitrogen up to 10 p.s.i accelerated column elution. Reverse phase preparative HPLC purifications were carried out using a Waters 2525 binary gradient pumping system at flow rates of typically 20 mL/min using a Waters 2996 photodiode array detector.

All solvents and commercial reagents were used as received.

Chemical names were generated using automated software such as the Autonom software provided as part of the ISIS Draw package from MDL Information Systems or the Chemaxon software provided as a component of MarvinSketch or as a component of the IDBS E-WorkBook.

X-Ray Powder Diffraction patterns were collected on a Philips X-Pert MPD diffractometer and analysed using the following experimental conditions (Method A), unless otherwise specified:
Tube anode: Cu
Generator tension: 40 kV
Tube current: 40 mA
Wavelength alpha1: 1.5406 Å
Wavelength alpha2: 1.5444 Å
Start angle [2θ]: 4
End angle [2θ]: 40
Continuous scan

Approximately 2 mg of sample under analysis was gently compressed on the XRPD zero back ground single obliquely cut silica sample holder. The sample was then loaded into the diffractometer for analysis.

Example 1—Preparation of the Compound of Formula A

A. 1-(4-Hydroxymethyl-benzyl)-1H-pyridin-2-one

4-(Chloromethyl)benzylalcohol (5.0 g, 31.93 mmol) was dissolved in acetone (150 mL). 2-hydroxypyridine (3.64 g, 38.3 mmol) and potassium carbonate (13.24 g, 95.78 mmol) were added and the reaction mixture was stirred at 50° C. for 3 hrs after which time the solvent was removed in vacuo and the residue taken up in chloroform (100 mL). This solution was washed with water (30 mL), brine (30 mL), dried (Na₂SO₄) and evaporated in vacuo. The residue was purified by flash chromatography (silica), eluent 3% MeOH/97% CHCl₃, to give a white solid identified as 1-(4-hydroxymethyl-benzyl)-1H-pyridin-2-one (5.30 g, 24.62 mmol, 77% yield).

[M+Na]⁺=238

B. 1-(4-Chloromethyl-benzyl)-1H-pyridin-2-one

1-(4-Hydroxymethyl-benzyl)-1H-pyridin-2-one (8.45 g, 39.3 mmol), dry DCM (80 mL) and triethylamine (7.66 ml, 55.0 mmol) were cooled in an ice bath. Methanesulfonyl chloride (3.95 ml, 51.0 mmol) was added and stirred in ice bath for 15 min. The ice bath was removed and stirring continued at rt temperature overnight. The reaction mixture was partitioned between DCM (100 mL) and saturated aqueous NH₄C1 solution (100 mL). The aqueous layer was extracted with further DCM (2×50 mL) and the combined organics washed with brine (50 mL), dried over Na₂SO₄, filtered and concentrated to give 1-(4-chloromethyl-benzyl)-1H-pyridin-2-one (8.65 g, 36.6 mmol, 93% yield) as a pale yellow solid.

[MH]⁺=234.1

C. Methyl 3-(methoxymethyl)-1-(4-((2-oxopyridin-1(2H)-yl)methyl)benzyl)-1H-pyrazole-4-carboxylate

Potassium carbonate (519 mg, 3.76 mmol) was added to a solution of methyl 3-(methoxymethyl)-1H-pyrazole-4-carboxylate (320 mg, 1.88 mmol; CAS no. 318496-66-1 (synthesised according to the method described in WO 2012/009009)) and 1-(4-(chloromethyl)benzyl)pyridin-2(1H)-one (527 mg, 2.26 mmol) in DMF (5 mL) and heated at 60° C. overnight. The reaction mixture was diluted with EtOAc (50 mL) and washed with brine (2×100 mL), dried over magnesium sulfate, filtered and reduced in vacuo. The crude product was purified by flash chromatography (40 g column, 0-100% EtOAc in isohexanes) to afford two regioisomers. The second isomer off the column was collected to afford methyl 3-(methoxymethyl)-1-(4-((2-oxopyridin-1(2H)-yl)methyl)benzyl)-1H-pyrazole-4-carboxylate (378 mg, 1.01 mmol, 53.7% yield) as a colourless gum.

[MH]⁺=368.2

D. 3-(Methoxymethyl)-1-(4-((2-oxopyridin-1(2H)-yl)methyl)benzyl)-1H-pyrazole-4-carboxylic acid

To methyl 3-(methoxymethyl)-1-(4-((2-oxopyridin-1(2H)-yl)methyl)benzyl)-1H-pyrazole-4-carboxylate (3.77 g, 10.26 mmol) in THF (5 mL) and MeOH (5 mL) was added 2M NaOH solution (15.39 ml, 30.8 mmol) and stirred at rt overnight. 1M HCl (50 mL) was added and extracted with EtOAc (50 mL). The organic layer was washed with brine (50 mL), dried over magnesium sulfate, filtered and reduced in vacuo to give 3-(methoxymethyl)-1-(4-((2-oxopyridin-1(2H)-yl)methyl)benzyl)-1H-pyrazole-4-carboxylic acid (1.22 g, 3.45 mmol, 33.6% yield) as a white powder.

[MH]⁺=354.2

E. 3-Fluoro-4-methoxy-pyridine-2-carbonitrile

To a large microwave vial, copper (1) cyanide (1.304 g, 14.56 mmol) was added to a solution of 2-bromo-3-fluoro-4-methoxypyridine (1 g, 4.85 mmol) in DMF (5 mL). The reaction vial was sealed and heated to 100° C. for 16 hrs. The reaction mixture was diluted with water (20 mL) and EtOAc (20 mL). The thick suspension was sonicated and required additional water (40 mL) and EtOAc (2×50 mL) with sonication to break-up the solid precipitated. The combined layers were filtered through a plug of celite and the organic layer isolated, washed with brine (50 mL), dried over magnesium sulfate, filtered and the solvent removed under reduced pressure to give a pale green solid identified as the desired compound 3-fluoro-4-methoxy-pyridine-2-carbonitrile (100 mg, 0.578 mmol, 12% yield)

F. (3-Fluoro-4-methoxy-pyridin-2-ylmethyl)-carbamic acid tert-butyl ester

3-Fluoro-4-methoxy-pyridine-2-carbonitrile (100 mg, 0.578 mmol) was dissolved in anhydrous methanol (10 mL, 247 mmol) and nickel chloride hexahydrate (14 mg, 0.058 mmol) was added followed by di-tert-butyl dicarbonate (255 mg, 1.157 mmol). The resulting pale green solution was cooled in an ice-salt bath to −5° C. and then sodium borohydride (153 mg, 4.05 mmol) was added portionwise maintaining the reaction temperature ˜0° C. The deep brown solution was left to stir at 0° C. and slowly allowed to warm to rt and then left to stir at rt for 3 hrs. The reaction mixture was evaporated to dryness at 40° C. to afford a black residue which was diluted with DCM (10 mL) and washed with sodium hydrogen carbonate (10 mL). An emulsion formed so the organics were separated via a phase separating cartridge and concentrated. The crude liquid was purified by chromatography eluting with EtOAc/iso-Hexane to afford the title compound, (3-fluoro-4-methoxy-pyridin-2-ylmethyl)-carbamic acid tert-butyl ester as a clear yellow oil (108 mg, 62% yield)

[MH]⁺=257

G. C-(3-Fluoro-4-methoxy-pyridin-2-yl)-methylamine hydrochloride salt

(3-Fluoro-4-methoxy-pyridin-2-ylmethyl)-carbamic acid tert-butyl ester (108 mg, 0.358 mmol) was taken up in iso-propyl alcohol (1 mL) and then HCl (6N in iso-propyl alcohol) (1 mL, 0.578 mmol) was added at rt and left to stir at 40° C. for 2 hrs. The reaction mixture was concentrated under reduced pressure and then triturated with ether, sonicated and then decanted to give a cream coloured solid (75 mg, 55% yield) identified as C-(3-fluoro-4-methoxy-pyridin-2-yl)-methylamine hydrochloride salt.

[MH]⁺=157

Example 1a-N-[(3-Fluoro-4-methoxypyridin-2-Yl)methyl]-3-(methoxymethyl)-1-({4-[(2-oxopyridin-1-yl)methyl]phenyl}methyl)pyrazole-4-carboxamide (Compound of Formula A)

3-(Methoxymethyl)-1-(4-((2-oxopyridin-1(2H)-yl)methyl)benzyl)-1H-pyrazole-4-carboxylic acid (825 mg, 2.34 mmol) and C-(3-fluoro-4-methoxy-pyridin-2-yl)-methylamine hydrochloride salt (450 mg, 2.34 mmol) were dissolved in DCM while cooling to 0° C. 1-Ethyl-3-(3-dimethylaminopropyl)carbodiimide hydrochloride (627.0 mg, 3.27 mmol), HOBt (378.8 mg, 2.80 mmol) and triethylamine (1.63 mL, 1182 mmol) were added while stirring, the mixture allowed to warm to rt and stirring continued for 20 hrs. Chloroform (50 mL) was added, the mixture was washed with saturated NaHCO₃(aq) and reduced in vacuo. The crude material was purified by chromatography eluting with methanol/DCM. The solvent was removed in vacuo and the resulting solid triturated with diethyl ether. The resulting solids were collected by filtration to afford the compound of Formula A.

[MH]⁺=492.0

NMR (CD₃OD) δ: 3.41 (3H, s), 4.03 (3H, s), 4.65 (2H, s), 4.72 (2H, d, J=2.3 Hz), 5.24 (2H, s), 5.37 (2H, s), 6.44 (1H, td, J=1.4, 6.8 Hz), 6.62 (1H, d, J=9.0 Hz), 7.18-7.22 (1H, m), 7.31-7.38 (4H, m), 7.56-7.60 (1H, m), 7.75 (1H, dd, J=1.9, 7.1 Hz), 8.18 (1H, s), 8.27 (1H, d, J=5.6 Hz) ppm.

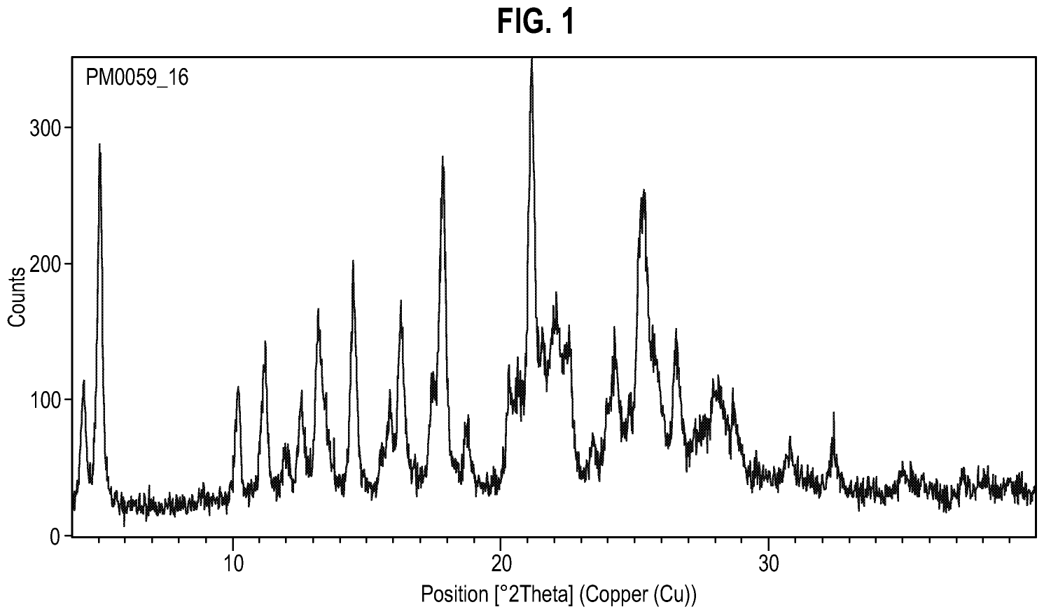

An XRPD diffractogram of the compound of Formula A resultant from the above procedure is shown in FIG. 1.

Peak position table:

| No. | Pos. [°2Th.] | Rel. Int. [%] |
|---|---|---|
| 1 | 4.436 | 32.36 |
| 2 | 5.0471 | 58.74 |
| 3 | 10.2255 | 43.07 |
| 4 | 11.2061 | 48.44 |
| 5 | 12.0101 | 16.4 |
| 6 | 12.5494 | 37.17 |
| 7 | 13.165 | 67.26 |
| 8 | 14.4984 | 38.94 |
| 9 | 15.8919 | 23.54 |
| 10 | 16.2983 | 34.56 |
| 11 | 17.4492 | 36.63 |
| 12 | 17.8564 | 71.49 |
| 13 | 18.6888 | 21.9 |
| 14 | 20.285 | 26.12 |
| 15 | 21.1598 | 100 |
| 16 | 22.04 | 87.76 |
| 17 | 22.5857 | 36.38 |
| 18 | 23.4408 | 14.33 |
| 19 | 24.3045 | 31.11 |
| 20 | 25.1655 | 78.97 |
| 21 | 25.3728 | 93.91 |
| 22 | 26.4946 | 56.79 |
| 23 | 27.991 | 76.91 |
| 24 | 28.7495 | 22.99 |
| 25 | 30.7611 | 13.4 |
| 26 | 32.413 | 17.2 |
| 27 | 37.2144 | 14.13 |
| 28 | 38.1171 | 14.14 |

Example 2—Preparation of a dosage form comprising the compound of Formula A

Blending and Roller Compaction

Equipment: Freund Vector TFC Lab Micro Roller Compactor and Granulator (the roller compactor and granulator are separate entities). The equipment parameters are below:

| Parameter | Range Used |
|---|---|
| Screw Speed (rpm) | 10.0-20.0 |
| Roll Speed (rpm) | 1.0-2.0 |
| Roll Force (kN) | 0.50-12.00 |
| Granulator Screen Size (mm) | 1 |

### Method

Two tablet formulations (Tablets A and B) were prepared according to the following method at 30 g blend scale to produce tablets having components in the amounts shown below.

| | | wt % (compared to the | |
|---|---|---|---|
| | | total mass of the tablet) | |
| | Component | Tablet A | Tablet B |
| Intragranular | The compound of | 42.5 | 47.5 |
| | Formula A | | |
| | Microcrystalline | 34.5 | 29.5 |
| | Cellulose | | |
| | (Avicel PH101) | | |
| | Croscarmellose | 4 | 4 |
| | Sodium (Ac-di-sol) | | |
| | Polyvinyl | 3 | 3 |
| | Pyrrolidone | | |
| | (Kollidon 30) | | |
| Extragranular | Microcrystalline | 15 | 15 |
| | Cellulose | | |
| | (Avicel PH102) | | |
| | Magnesium | 1 | 1 |
| | Stearate | | |

For each of the tablets, blends were prepared by passing the intragranular components through a 355 m sieve at a suitable scale for the scope of the roller compactor in a glass vessel using a Turbula Blender at 34 rpm. The blend was then run through the roller compactor using the parameters described above. The ribbons produced were collected into a suitably sized container. The collected ribbons were then subjected to the granulator fixed with a 1 mm screen and the resultant granules were collected for further downstream processing.

Tabletting

Equipment: RIVA Mini single station Tablet Press. The equipment parameters are shown below:

| Parameter | Range Used |
|---|---|
| Tooling Diameter | 8 mm round |
| Force (kN) | 6-10 |
| Fill Weight (mg) | 178-300 |

The granules were subsequently blended with their extragranular excipients, respectively. The extragranular excipients were prepared by screening through a 355 μm sieve in a glass vessel using a Turbula Blender at 34 rpm. The target tablet weight was then dispensed and manually compressed into tablets. Tablet A was compressed at 7.2 to 8.8 kN compression force. Tablet B was compressed at 6.9 to 7.7 kN compression force.

The tablets were found to be robust. Tablets A and B were subsequently submitted for long-term stability testing.

The production of tablets per the method described above has been scaled to 180g with a roller compaction time of approximately 60 minutes.

Example 3—Comparison of the compound of Formula A with a C1 inhibitor (C1-INH)

Aim: To identify the biochemical and biophysical properties of the compound of Formula A that contribute to its optimal efficacy in controlling the Kallikrein Kinin System in plasma. These properties are then compared to C1-INH as a therapeutic benchmark for HAE.

### Methods

Plasma kallikrein inhibitory activity in vitro was determined using standard published methods (see e.g. Johansen et al., Int. J. Tiss. Reac. 1986, 8, 185; Shori et al., Biochem. Pharmacol., 1992, 43, 1209; Sturzebecher et al., Biol. Chem. Hoppe-Seyler, 1992, 373, 1025). Human plasma kallikrein (Protogen) was incubated at 25° C. with the fluorogenic substrate H-DPro-Phe-Arg-AFC and various concentrations of the test compound. Residual enzyme activity (initial rate of reaction) was determined by measuring the change in optical absorbance at 410 nm and the IC₅₀ value for the test compound was determined.

The rate of formation of the enzyme-inhibitor complex (Kₒₙ) was determined using purified PKa rapidly mixed with a solution containing fluorogenic substrate and a concentration range of inhibitor. The time-dependent establishment of inhibition was then used to calculate the rate of formation of the enzyme-inhibitor complex for each concentration of inhibitor. The Kₒₙ was calculated by plotting the rate of inhibition versus the inhibitor concentration. Data in Table 1 are presented in μM⁻¹ sec⁻¹.

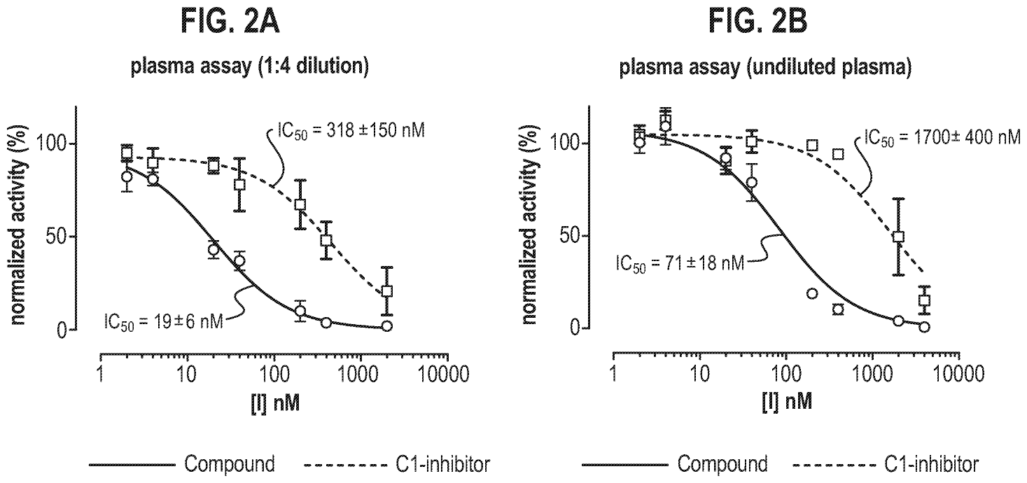

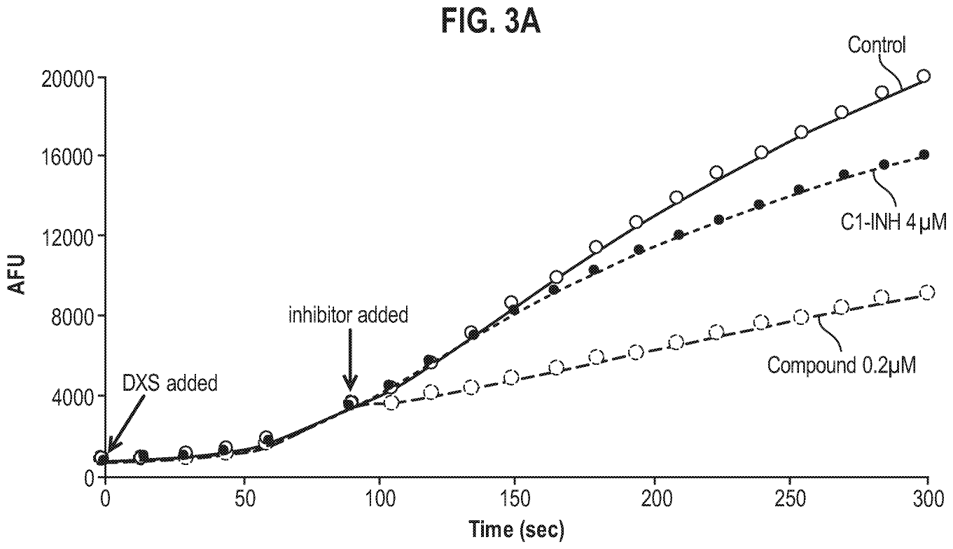

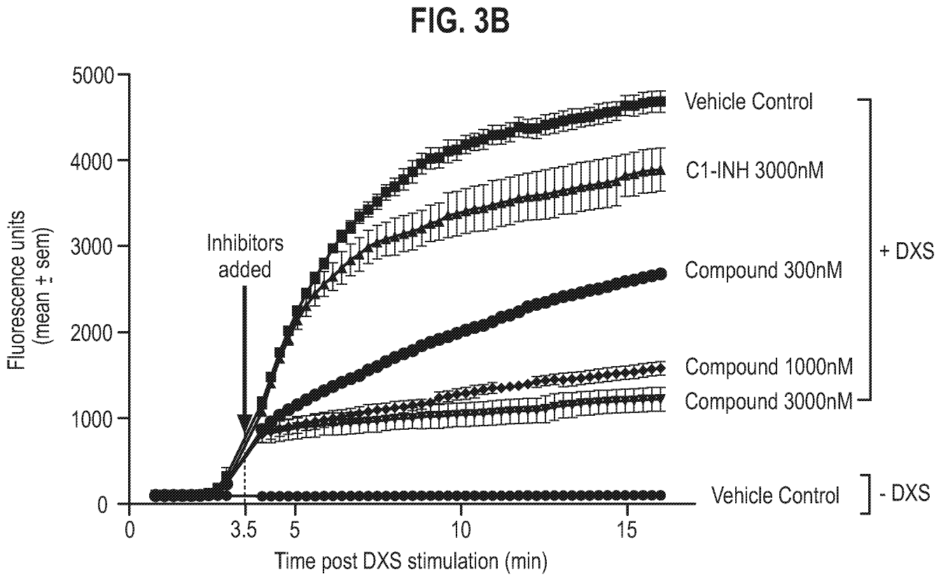

Catalytic activity of PKa in dextran sulfate-activated (DXS, Sigma; 10 μg/ml) plasma (1:4 diluted or undiluted, VisuCon-F control plasma, Affinity Biologicals Inc) was determined by the time-dependent hydrolysis of fluorogenic substrate. For IC₅₀ and efficacy determination, the compound of Formula A or C1-INH (Sigma Cat #E0518) were added either before (FIGS. 2A and 2B) or after (FIG. 3A) the addition of DXS to the plasma.

DXS-activated cleavage of HK in undiluted plasma was performed in the absence or presence of 300 nM PKa inhibitor and quantified by SDS-PAGE gel electrophoresis, using 7.5% Criterion TGX Precast gels (Biorad). Transfer was made onto Immunobilon-FL PVDF membrane. Image analysis was done using the LICOR imaging system. Mouse monoclonal anti-HK antibody (MAB15692, R&D systems) was used for traditional immunoblotting. Data presented as % of HK remaining after 20 min incubation with DXS compared to HK levels in unactivated plasma (Table 1).

Plasma free fraction was determined using “Rapid Equilibrium Dialysis” system (Thermo Scientific), test compounds were prepared at 5 μM in neat human plasma and dialysed against phosphate buffer for 5 hrs at 37° C. Quantification of the compound partitioned in two chambers of the dialysis device was performed via LCMS/MS. Fraction of compound unbound to plasma proteins presented as % of total.

The ability of the compound to inhibit the enzyme activity of pre-activated plasma was assessed by addition of the compound after DXS stimulation. Aliquots of plasma (20 μL) were mixed with a 2.5 μL solution containing 1,300 mM fluorogenic substrate (H-DPro-Phe-Arg-AFC) and a 2.5 μL solution of dextran sulphate (DXS; 100 μg/mL) which acted as an activator of the plasma kallikrein-kinin pathway. Enzyme activity was immediately measured by monitoring the accumulation of fluorescence liberated from the substrate by substrate cleavage over 16 minutes. At 3.5 minutes post DXS addition 5 μl of inhibitors or water control are were added into each well. The compound was tested at concentrations of 300, 1000 and 3000 nM. C1-INH at a concentration of 3000 nM and vehicle controls were also included. Data are presented in FIG. 3B.

Results:

As shown in FIG. 2, in assays using the fluorogenic substrate, the compound of Formula A appears to be a highly potent inhibitor of PKa with 17-fold and 20-fold potency vs. exogenously added C1-INH in diluted plasma (FIG. 2A) and undiluted plasma (FIG. 2B), respectively.

Table 1 showing the biochemical profile of the therapies tested in this example.

| | IC₅₀ | IC₅₀ | | Plasma | |
|---|---|---|---|---|---|
| | purified | plasma | | Free | HK |
| | enzyme | enzyme | Kₒₙ | Fraction | protection |
| | nM | nM | (μM⁻¹ sec⁻¹) | % | % |
| C1-INH | 50 | 1700 | 0.04 | | |
| Compound of | 6 | 71 | 11 | 25 | 85 |
| Formula A | | | | | |

FIG. 3A shows a comparison of the effects of the two inhibitors: compound of Formula A and C1-INH, on plasma kallikrein activity in plasma (diluted 1:4) activated with DXS. Both inhibitors were added at concentrations ten times their IC₅₀ to plasma approximately 100 seconds after the addition of the DXS.

FIG. 3B shows that addition of the compound of Formula A after the activation of plasma causes rapid and dose dependent inhibition of enzyme activity compared to the slower action of C1-INH.

Table 2 shows the biochemical potency and selectivity of the compound of Formula A against human isolated enzymes using literature methods as for the above-described in vitro plasma kallikrein assay.

| The compound | Ki (nM) |
|---|---|
| Plasma Kallikrein | 3.02 |
| Selectivity vs PKa | Fold |
| Tissue Kallikrein (KLK1) | >6000 |
| Factor XIa | >6000 |
| Factor XIIa | >6000 |
| Thrombin | >6000 |
| Trypsin | >6000 |

Example 4—Phase I Single Ascending Dose Study in Healthy Males and Food Effect of the Compound

Aim: To evaluate the pharmacodynamic (PD) effects of the compound of Formula A when orally administered using ex vivo whole plasma assays for plasma kallikrein catalytic activity and HK cleavage, in samples from a Phase 1 Single Ascending Dose Study in healthy adult males. Also, an aim was to investigate safety, tolerability and pharmacokinetic (PK) effects of the compound of Formula A when orally administered.

Methods:

This study was a randomized, double-blind, placebo-controlled single ascending dose (SAD) and crossover studies for food effect and capsule/tablet formulations.

64 healthy male participants (n=6 active, 2 placebo per cohort, 8 SAD cohorts) were administered single ascending doses of the compound of Formula A: 5, 10, 20, 40, 80, 160, 300 or 600 mg in a capsule.

8 participants were administered 100 mg the compound of Formula A in a crossover study of the capsule and a tablet formulation.

12 participants were administered 600 mg the compound of Formula A in a food effect crossover study.

Samples for pharmacokinetic (PK) and PD assessment were taken at repeated intervals over 48 hours.

Plasma samples used for PK assessment were analysed using a validated liquid chromatography tandem mass spectrometry (LC MS/MS) method.

PD measurements were determined in dextran sulfate (DXS) stimulated undiluted plasma using a fluorogenic enzyme assay and capillary based HK cleavage immunoassay.

Catalytic activity of PKa in DXS-stimulated (Sigma; 10 μg/mL) plasma samples from the compound of Formula A phase 1 study was determined by the time-dependent hydrolysis of fluorogenic substrate in all samples from all parts of the study.

The time until appearance of detectable amidolytic enzyme activity in DXS-stimulated plasma (lag time) was calculated from the catalytic activity assay. The detection sensitivity of the rate of catalytic activity in plasma based on using a Spark (Tecan) fluorimeter is a fluorescence increase to reach 1ΔF unit/sec.

DXS-stimulated cleavage of HK, in undiluted plasma was quantified by capillary-based immunoassay on the Wes System (ProteinSimple) using monoclonal anti-HK antibody and chemiluminescence-based detection. Plasma kallikrein mediated HK cleavage in undiluted citrated human plasma was induced by contact system activation with DXS (6.25 μg/ml) at 4° C. in selected samples from the SAD phase.

DXS-stimulated cleavage of plasma prekallikrein and Factor XII (FXII) were quantified by capillary-based immunoassay on the Wes System (ProteinSimple) analogously.

Results:

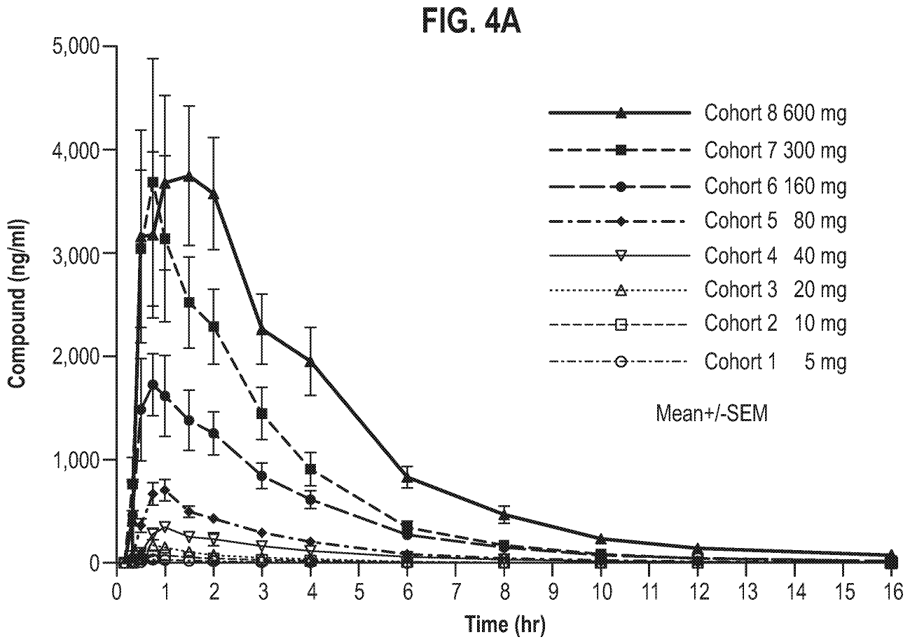

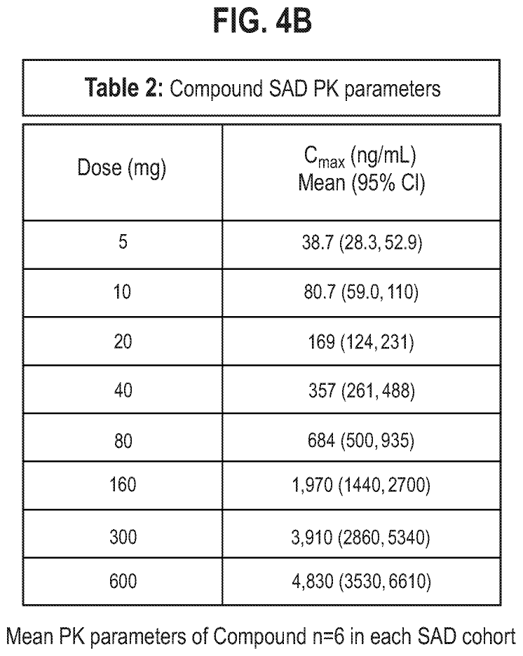

FIG. 4A shows the plasma concentrations of the compound of Formula A from 0 to 24 hours post-dose. As can be seen, when orally administered, the compound of Formula A achieved rapid and dose-dependent plasma exposure over the range of doses tested from 5 mg to 600 mg. FIG. 4A shows the concentration curves and FIG. 4B shows the Cₘₐₓ for each SAD cohort. The compound of Formula A was administered as a capsule formulation and the subject was in the fasted state.

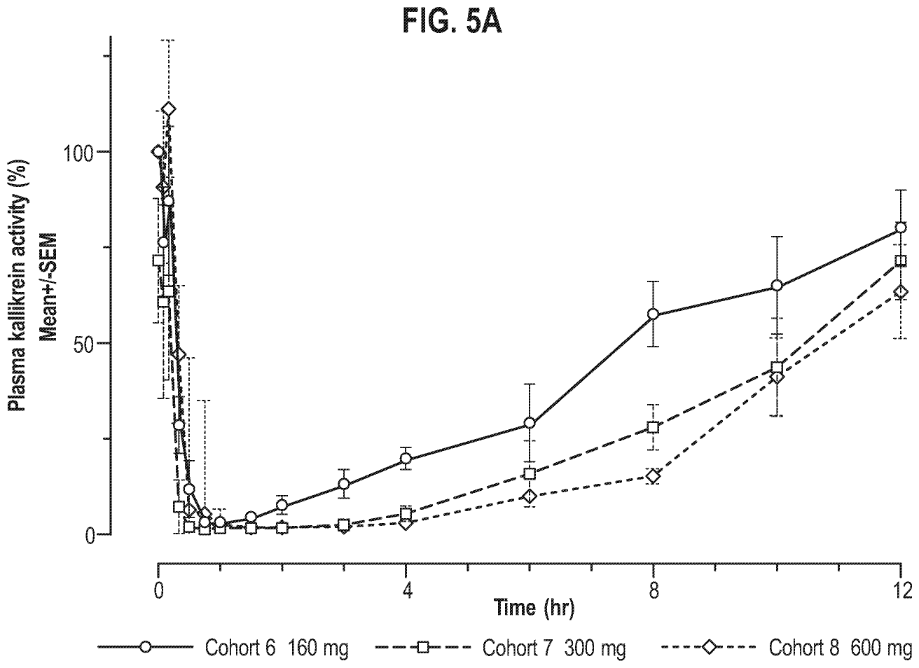

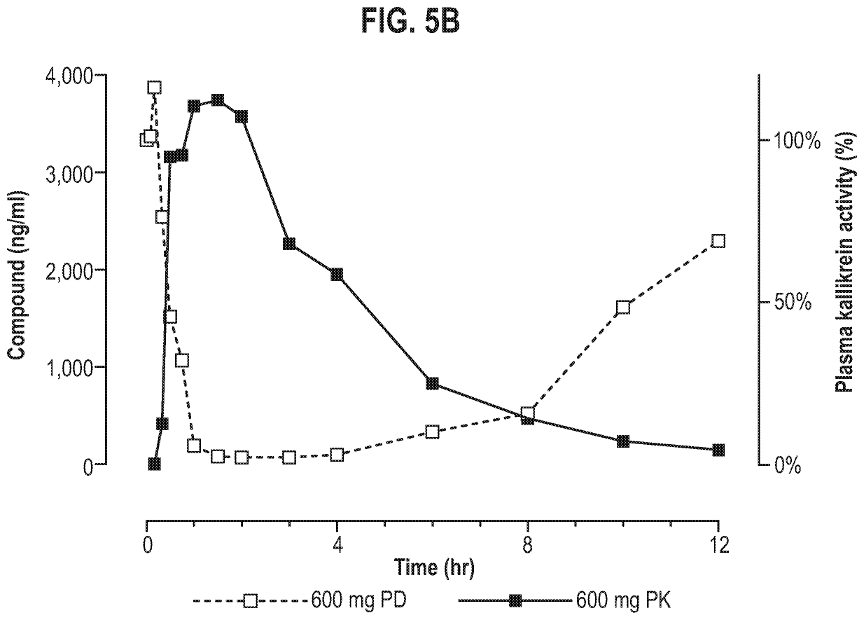

FIG. 5A shows enzyme assays in activated undiluted plasma performed on samples from cohorts 6, 7, and 8. Doses 160 mg and above demonstrated >90% average inhibition of plasma kallikrein catalytic activity between 45 min and 2 hr for cohort 6, between 20 min and 4 hr for cohort 7. A 600 mg dose (cohort 8) provided >90% inhibition of plasma kallikrein catalytic activity between 30 min and 6 hr post-dose and >50% inhibition for 10 hr (FIG. 5B).

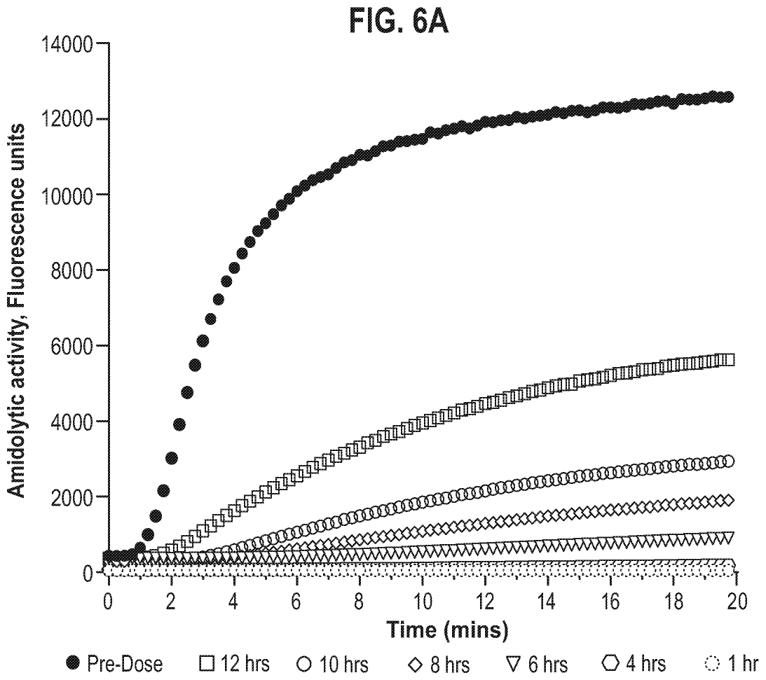

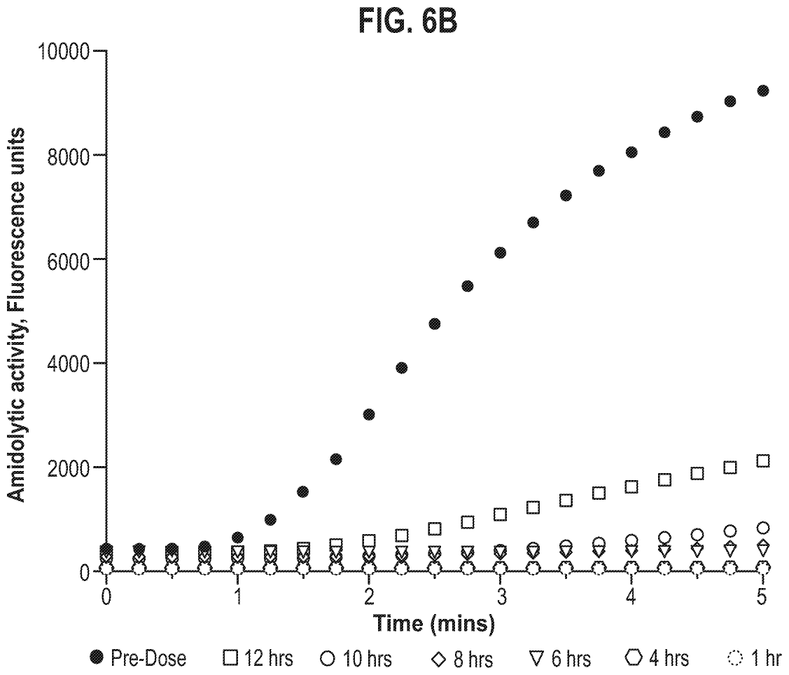

The kinetic fluorescent measurements from the undiluted plasma enzyme assay can be plotted as assay progression curves (FIGS. 6A and 6B). These curves highlight that the compound of Formula A not only has an inhibitory effect on enzyme activity but also increases the time until appearance of catalytic activity during contact system activation (lag time). At early time points post-dose administration, plasma samples did not display detectable catalytic activity even after prolonged activation with the potent activator DXS. In this test, the subject was administered with 600 mg dose in a tablet formulation.

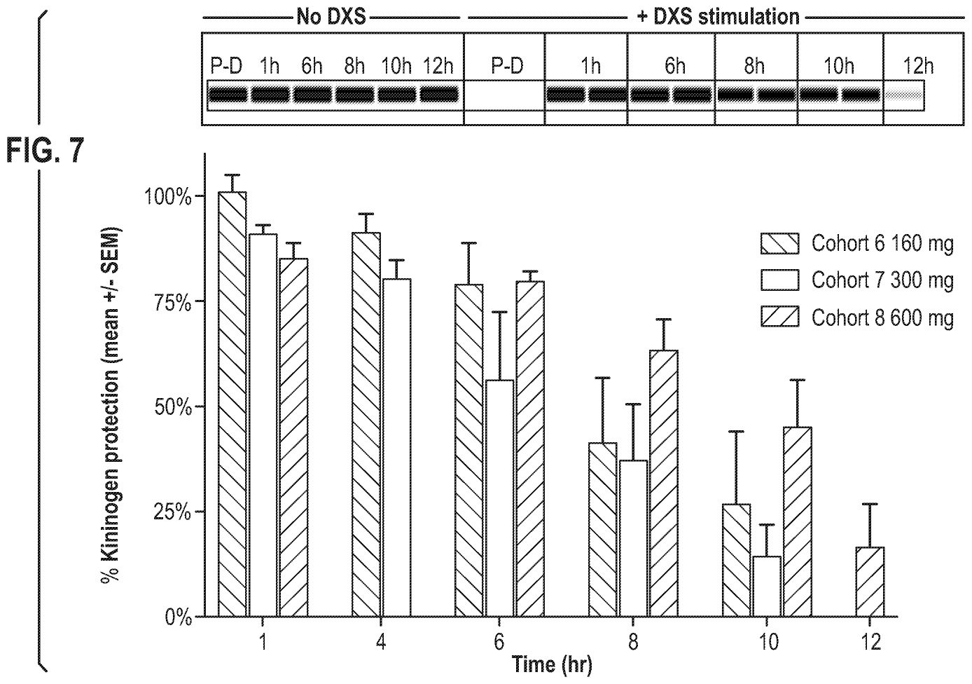

FIG. 7 shows the mean percent HK protection in DXS-activated undiluted plasma (SAD cohort 6 (160 mg), 7 (300 mg) and 8 (600 mg)). As shown, all three doses of the compound of Formula A were able to inhibit plasma kallikrein catalytic activity above 90% for a period of time. The duration of these PD effects was dose proportional. The compound of Formula A is shown to protect HK from DXS-activated cleavage in undiluted plasma for at least 10 hr following a single 600 mg dose.

In FIG. 7, the representative WES system gel image was generated in duplicate undiluted plasma samples+/−DXS activation from a single subject in cohort 8 who received 600 mg of the compound of Formula A compared with pre-dose (P-D).

In FIG. 7, HK cleavage was evaluated following DXS activation of undiluted plasma samples at selected time points from cohorts 6 to 8. Data are expressed as Mean+/−SEM, n=6.

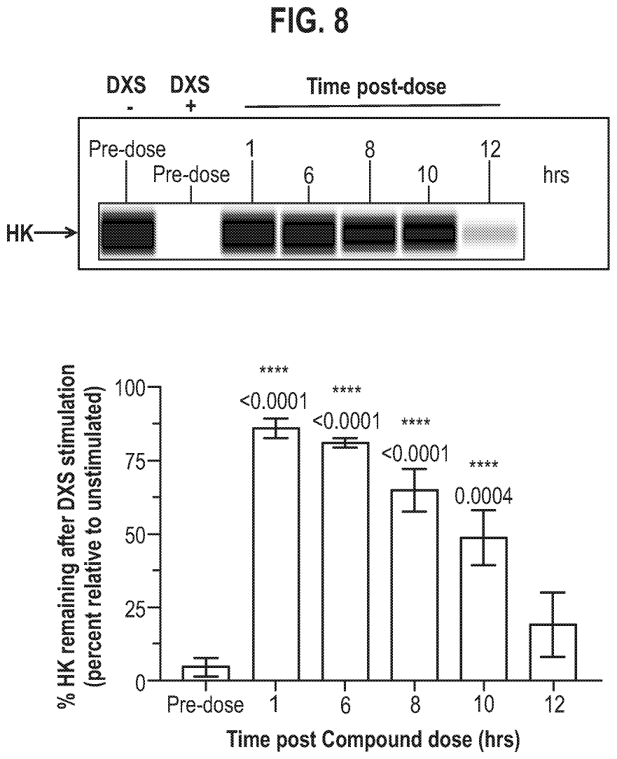

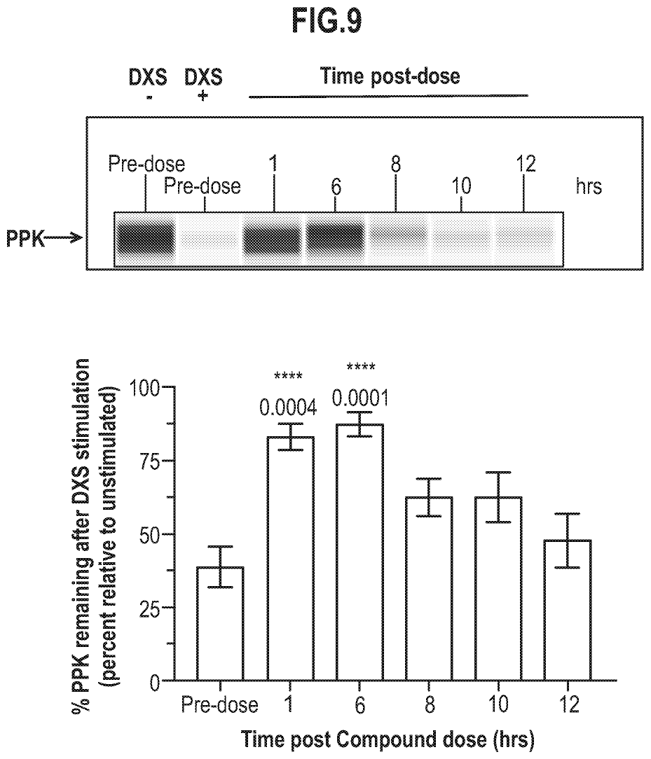

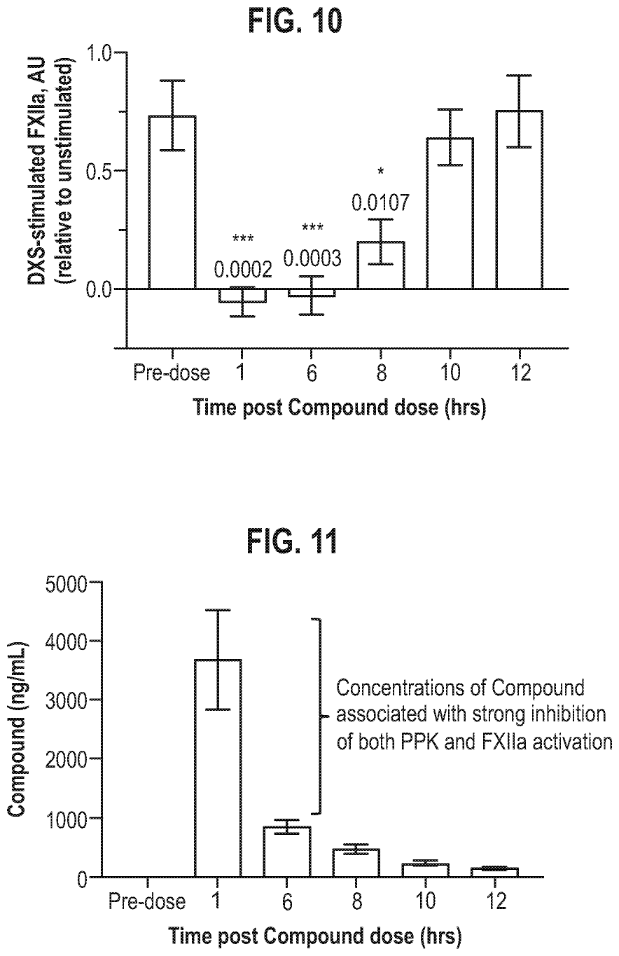

To assess whether the compound of Formula A also reduced the generation of plasma kallikrein and Factor XIIa, immunoassays were used to quantify levels of contact system proteins in DXS-activated plasma at pre-dose and up to 12 hrs post-dose of 600 mg orally administered in capsules. The results from these assays are shown in FIGS. 8 to 11 and shows that the compound of Formula A not only reduces HK cleavage but also reduces PPK cleavage and reduces the generation of FXIIa. These results suggest that the compound of Formula A inhibits the contact activation system via interruption of the positive feedback loop mediated by PKa stimulated activation of FXII.

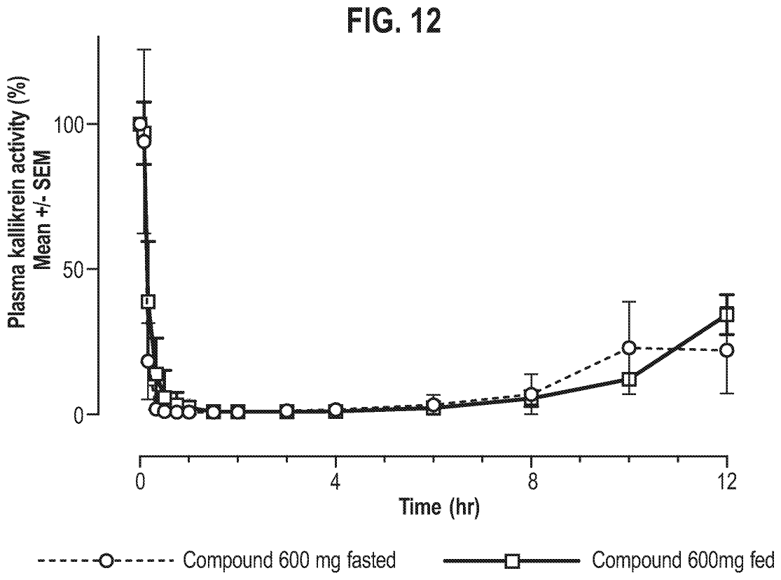

FIG. 12 shows that no significant food-effect was observed on the pharmacodynamic (PD) profile of a 600 mg tablet provided in fed and fasted state. As can be seen, the PD effects are rapidly observed in fed and fasted state with plasma kallikrein inhibition of >90% achieved by 30 minutes in both states.

No serious adverse events were reported in the phase I trial. There were also no tolerability signals. No subjects withdrew from the trial.

These data demonstrate that the compound of Formula A has an inhibitory effect on the bradykinin and contact activation systems. As discussed above, these pharmacodynamic effects are implicated in disorders such as HAE. These data also show that the compound of Formula A has a pharmacokinetic profile suitable for oral administration.

Example 5—Immunoassays Investigating the Compound of Formula a in the Protection of High Molecular Weight Kininogen (HK) from PKa-Mediated Cleavage in HAE and Control Plasma

Method:

High molecular weight kininogen (HK) cleavage in undiluted citrated human plasma was induced by contact system activation with dextran sulfate (DXS, Sigma #31395-10 G; 6.25 μg/ml) on wet ice. Pooled normal (CONTROL) human plasma (VisuCon-F Frozen Normal Control plasma) was purchased from Affinity Biologicals Inc. A working stock of 10 mM of the compound of formula A (“the compound”) in DMSO was prepared and diluted in 1× PBS to the respective final concentrations described. HAE plasma was obtained from HAE subjects (n=6) and C1-inhibitor deficiency was confirmed by western blotting. Protection of HK from PKa mediated cleavage in DXS-stimulated whole undiluted plasma was then determined by two methods; traditional western blotting and a semi-automated capillary-based immunoassay.

Western blotting: SDS-PAGE gel electrophoresis was done using 7.5% Criterion TGX Precast gels (Bio-rad). Transfer was made onto Immobilon-FL PVDF membrane. Image analysis was performed using the LICOR imaging system. Mouse monoclonal anti-human HK antibody (MAB15692, R&D systems) was used for traditional immunoblotting.

Capillary-Based Immunoassay on the WES System (ProteinSimple):

Preparation of samples: Combine one part 5× fluorescent master mix with four parts of the 1:200 plasma sample. Vortex to mix. Heat the samples+fluorescent master mix and the biotinylated ladder at 95° C. for 5 minutes, vortex, and load onto the WES plate. Monoclonal anti-human HK antibody was used for this chemiluminescence-based detection method using the Wes System (ProteinSimple).

Analysis: Collect the peak area measurement obtained in the Compass software (cbz file) for the full-length HK molecular weight of the respective time-point sample with DXS-induced activation. The peak area is defined as the area calculated for the spectral peak profile for HK. To measure the plasma kallikrein inhibition by the compound, the percent full-length HK detected was calculated.

Results:

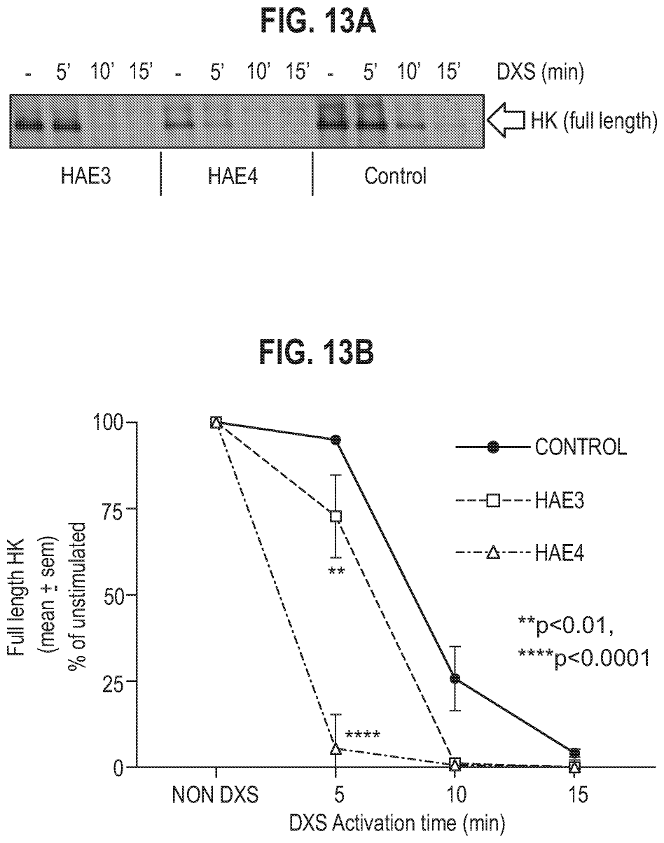

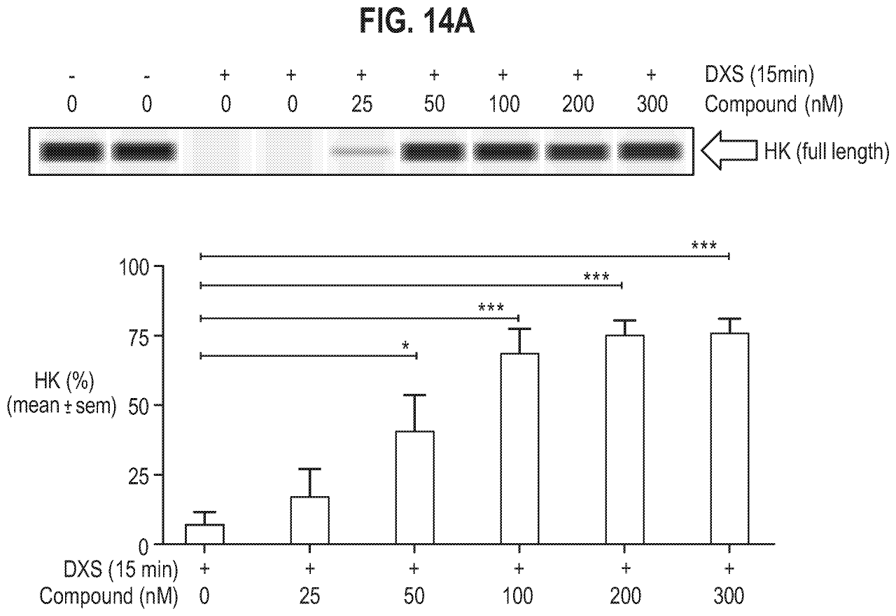

FIGS. 13A and 13B show the time course of dextran sulfate-activated cleavage of HK in HAE whole undiluted plasma determined using western blotting, and a representative blot.

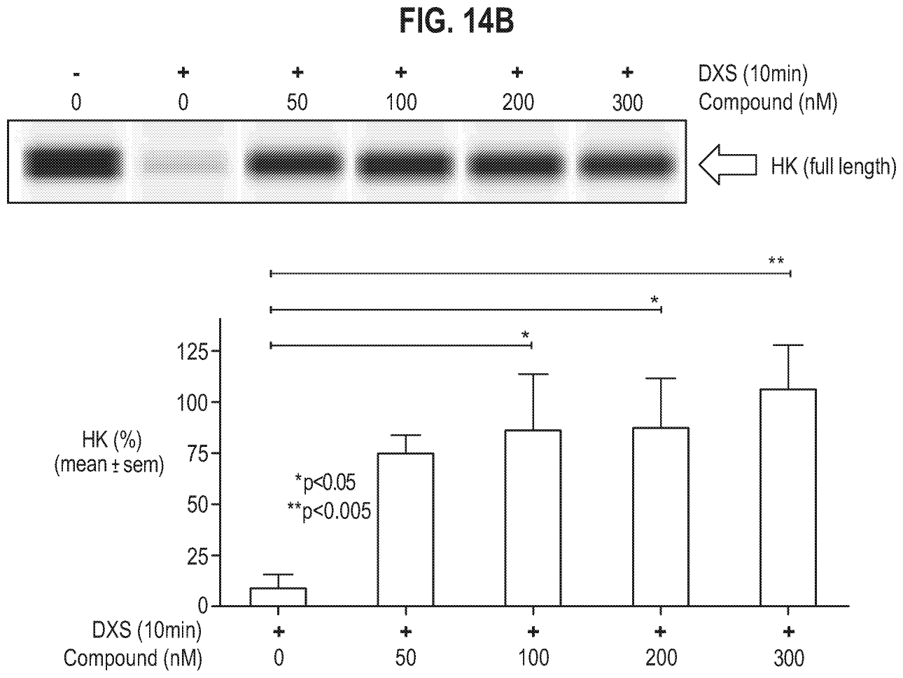

FIGS. 14A and 14B shows a representative WES system gel image and that the compound of Formula A provides dose dependent protection against HK cleavage in both HAE and healthy control plasma stimulated with dextran sulfate determined by capillary-based immunoassay using the WES system.

Example 6—Phase 2 Study of the Compound of Formula A

Aim: To evaluate the efficacy and safety of the compound of Formula A in the on-demand treatment of angioedema attacks in adult subjects with hereditary angioedema type I or II.

Methods:

The study was a randomized, double-blind, placebo-controlled, phase 2, cross-over clinical trial evaluating the efficacy and safety of the compound of formula A (“the compound”), an oral plasma kallikrein inhibitor, in the on-demand treatment of angioedema attacks in adult subjects with hereditary angioedema type I or II (EudraCT number: 2018-004489-32).

Objectives:

Primary Objective:
To investigate the efficacy of the compound compared to placebo in halting the progression of a peripheral or abdominal attack of hereditary angioedema (HAE).

Secondary Objectives:
To investigate the safety and tolerability of the compound.
To investigate the pharmacokinetic (PK) profile of the compound when taken during the intercritical period between HAE attacks.
To investigate the pharmacodynamic (PD) profile of the compound in reducing the concentration of residual cleaved high molecular weight kininogen (HK) during the intercritical period between HAE attacks.
To investigate the PD profile of the compound in reducing activated plasma enzyme activity during the intercritical period between HAE attacks.

Setup:

This was a phase 2, two-part, two-sequence, two-period (2×2) cross-over clinical trial. Subjects with HAE type I or II were recruited through HAE treatment centres in Europe and US.

In Part 1, subjects received a single oral dose of 600 mg of the compound to investigate the safety, PK and PD of the compound during the intercritical period between HAE attacks.

Eligible adult subjects ≥18 years old underwent a screening assessment for study inclusion and to receive study drug, followed by a 4 h, in-clinic, safety and PK/PD assessment.

In Part 2, the subjects were randomized 1:1 to 2 treatment sequences. This part of the study was conducted away from the clinic or hospital. In Sequence 1 (study arm 1) subjects received a single dose of 600 mg of the compound to treat the first eligible HAE attack. Following resolution of this attack, subjects received a single dose of placebo to treat the second eligible HAE attack.

In Sequence 2 (study arm 2) subjects received a single dose of placebo to treat the first eligible HAE attack. Following resolution of this attack, subjects received a single dose of 600 mg of the compound to treat the second eligible HAE attack.

A minimum of 48-hour washout period was required between each dose of study drug.

Laryngeal or facial attacks were not eligible for treatment. HAE attacks must have been treated within the first hour of onset and before reaching severe on the global attack severity scale. Subjects must also have been able to identify the start of a HAE attack. Upon onset of the eligible HAE attack, subjects notified the dedicated study physician or qualified designee with a description of the HAE attack. The dedicated study physician or qualified designee confirmed eligibility of the HAE attack and agreed to study drug being administered. HAE attacks required documentation, on the Subject Diary, of attack location, attack symptoms, time of onset, attack severity, and time of last substantial meal prior to dosing. Subjects took study drug, as instructed, and completed timed assessments of their HAE attack symptoms for a 48 h period as documented below in Table 3. The dedicated study physician or qualified designee contacted the subject within 24 h of the eligible HAE attack to confirm the subject's safety and wellbeing. Subjects were instructed to contact the dedicated study physician or qualified designee in case of any safety concerns. In the case of hypersensitivity, subjects contacted the dedicated study physician or qualified designee or contact the nearest emergency service. The dedicated study physician or qualified designee was available 24 h/day and 7 days/week to receive subject calls.

TABLE 3
Frequency of Subject Assessment

| Time Period | Frequency | Allowed Time |
|---|---|---|
| following Study | of Subject | Window for |
| Drug Administration | Assessment* | Assessment |
| 0 h-4 h | Every 30 min | None |
| 4 h-12 h | Every 1 h | +/−15 min |
| 12 h-24 h | Every 3 h | +/−30 min |
| 36 h | Once | +/−60 min |
| 48 h | Once | +/−60 min |

*In the event that conventional attack treatment was used, the subject performed assessments every 30 min for 4 h following first administration of conventional attack treatment. After this, the subject reverted back to original frequency of assessments based on time of study drug administration.

Subjects returned to the clinic following the first HAE attack, prior to the second HAE attack, to undergo safety checks including adverse event (AE) reporting, vital sign recording, and Subject Diary review.

Once two HAE attacks were treated in Part 2, the subject returned to the clinic to undergo final safety checks including AE reporting, vital sign recording and blood sampling for laboratory safety measurements.

Conventional attack treatment was permitted after 4 h, or earlier as warranted, following study drug intake, provided HAE attack symptoms were judged severe enough by the subject to require treatment as per the subject's usual treatment regimen, or are deemed ineligible for study drug treatment, or were associated with laryngeal or facial symptoms. Prior to use of conventional attack treatment, subjects notified the dedicated study physician or qualified designee who confirmed conventional treatment was appropriate per protocol and subject report of symptom severity. Subjects were permitted to treat their HAE attacks with their conventional attack treatment (pdC1INH or rhC1INH intravenous [iv] or icatibant).

Investigational Medicinal Product:

The compound of formula A-100 mg film-coated tablet. These contained the following excipients: microcrystalline cellulose, croscarmellose sodium, povidone, magnesium stearate; the aesthetic coating contains hypromellose, lactose monohydrate, titanium dioxide and triacetin.

Placebo to the compound 100 mg film-coated tablet. These contained microcrystalline cellulose, colloidal silicon dioxide, sodium starch glycolate, and sodium stearyl fumarate and are film-coated; the aesthetic coating contains hypromellose, lactose monohydrate, titanium dioxide and triacetin.

No study drug dose modifications were allowed in this study.

Number of Subjects:

68 subjects were enrolled into the study to ensure 50 subjects complete the study. 53 subjects completed Part 2.

Population:

The study population included male and female subjects 18 years of age or older with HAE type I or II.

Inclusion Criteria:
1. Male or female adult subjects 18 years of age and older.
2. Confirmed diagnosis of HAE type I or II at anytime in the medical history:
   a. Documented clinical history consistent with HAE (subcutaneous or mucosal, nonpruritic swelling episodes without accompanying urticaria) AND
   b. C1-esterase inhibitor (C1-INH) antigen or functional level <40% of the normal level. Subjects with antigen or functional C1-INH level 40-50% of the normal level were enrolled if they also have a C4 level below the normal range and a family history consistent with HAE type I or II.
3. At least 3 documented HAE attacks in the past 93 days, as supported by medical history.
4. Access to and ability to use conventional attack treatment for attacks of HAE.
5. Adequate organ functions as defined below:
   a. Hemoglobin within normal range;
   b. International normalized ratio (INR)<1.2;
   c. Activated partial thromboplastin time (aPTT)≤upper limit of normal (ULN);
   d. Creatinine <1× ULN;
   e. Creatinine clearance (CrCI)≥60 mL/min;
   f. Alanine aminotransferase (ALT)≥2× ULN;
   g. Aspartate aminotransferase (AST)≥2× ULN;
   h. Total bilirubin≥1.5× ULN;
   i. Leucocytes≥1.5× ULN;
   j. Thrombocytes≥1.5× ULN.
6. Female of childbearing potential must have agreed to use highly effective birth control from the Screening visit until the end of the trial follow-up procedures.
Highly effective methods of birth control include:
   a. Progestogen-only hormonal contraception associated with inhibition of ovulation:
   oral/injectable/implantable.
(Hormonal contraception that contains estrogen was excluded per exclusion criterion 3).
   b. Intrauterine device (IUD).
   c. Intrauterine hormone-releasing system (IUS).
   d. Bilateral tubal occlusion.
   e. Vasectomised partner (provided that the partner was the sole sexual partner of the female subject of childbearing potential and that the vasectomised partner received medical assessment of the surgical success).
   f. Sexual abstinence (this method is not acceptable in Switzerland).
   Note: Sexual abstinence was only be considered a highly effective method if it is defined as refraining from heterosexual intercourse. The reliability of sexual abstinence needed to be evaluated in relation to the duration of the clinical trial and the preferred and usual lifestyle of the subject.
7. Females of non-childbearing potential, defined as surgically sterile (status post hysterectomy, bilateral oophorectomy, or bilateral tubal ligation) or post-menopausal for at least 12 months, did not require contraception during the study.
8. Males with female partners of childbearing potential must have agreed to be abstinent or else use a highly effective method of birth control as defined in inclusion criterion 6 from the Screening visit until the end of the trial follow-up procedures.
9. Provide signed informed consent and were willing and capable of complying with study requirements and procedures.

Exclusion Criteria:
1. Any concomitant diagnosis of another form of chronic angioedema, such as acquired C1 inhibitor deficiency, HAE with normal C1-INH (also known as HAE type Ill), idiopathic angioedema, or angioedema associated with urticaria.
2. Current use of C11NH, androgens, lanadelumab or tranexamic acid for HAE prophylaxis.
3. Use of angiotensin-converting enzyme (ACE) inhibitors or any estrogen-containing medications with systemic absorption (such as oral contraceptives or hormonal replacement therapy) within 93 days prior to initial study treatment.
4. Use of androgens (e.g. stanozolol, danazol, oxandrolone, methyltestosterones, testosterone) or antifibrinolytics within 30 days prior to initial study treatment.
5. Use of lanadelumab within 10 weeks prior to initial study treatment.
6. Use of strong CYP3A4/CYP2C9 inhibitors and inducers during participation in the trial.
Note: These medications include but are not limited to the following: cobicistat, conivaptan, itraconazole, ketoconazole, posaconazole, voriconazole, ritonavir, boceprevir, telaprevir, troleandomycin, clarithromycin, carbamazepine, enzalutamide, mitotane, phenytoin, phenobarbital, fluconazole, isoniazid, metronidazole, paroxetine, sulfamethoxazole, rifampicin, St. John's Wort, diltiazem, idelalisib, nefazodone and nelfinavir.
7. Clinically significant abnormal electrocardiogram (ECG) at Visit 1 and pre-dose at Visit 2. This includes, but is not limited to, a QTcF >470 msec (for women) or >450 msec (for men), a PR >220 msec or ventricular and/or atrial premature contractions that are more frequent than occasional and/or occur as couplets or higher in grouping.
8. Any clinically significant history of angina, myocardial infarction, syncope, clinically significant cardiac arrhythmias, left ventricular hypertrophy, cardiomyopathy, or any other cardiovascular abnormality.
9. Any other systemic dysfunction (e.g., gastrointestinal, renal, respiratory, cardiovascular) or significant disease or disorder which, in the opinion of the Investigator, would have jeopardized the safety of the subject by taking part in the trial.
10. History of substance abuse or dependence that would interfere with the completion of the study, as determined by the Investigator.
11. Known lactose allergy or intolerance.
12. Known hypersensitivity to the compound or placebo or to any of the excipients.
13. Participation in an interventional investigational clinical study within 93 days or within 5 half-lives of the last dosing of investigational drug (whichever is longer) prior to initial study treatment.
14. Any pregnant or breast-feeding subject.

Assessments:

Part 1: Blood samples for PK and PD measurements were collected at the following timepoints: Pre-dose (oh), 15 min, 30 min, 45 min, 1 h, 1.5 h, 2 h, 3 h, and 4 h post-dose. Vital signs (systolic blood pressure [SBP], diastolic blood pressure [DBP], pulse rate [PR], respiratory rate [RR] and body temperature) were measured at pre-dose (0 h), 1 h, and 4 h post-dose. Samples for post-treatment safety laboratory assessments were taken with the 4 h PK/PD samples.

Part 2: Following study drug intake, subject assessments of overall HAE attack severity and change in HAE attack severity took place for a 48 h period as documented in Table 3 above.

Efficacy Variables:

Time to use of conventional attack treatment was assessed. The subject diary captured the efficacy endpoints including time to use of conventional attack treatment and HAE attack severity.

Overall HAE attack severity was assessed on a 5-point Likert scale (5LS) scored as none, mild, moderate, severe and very severe.

Change in HAE attack severity was assessed using a 7-point transition question (7TQ), scored as Much better/Better/A little better/No change/A little worse/Worse/Much worse.

The type of HAE attack symptoms (abdominal pain, skin pain and skin swelling) was each assessed on a 100 mm visual analogue scale (VAS) anchored at 0 (none) and 100 (very severe).

Safety Variables:
AEs, including serious adverse events (SAEs).
Laboratory test results (clinical chemistry, hematology, coagulation, and urinalysis).
Vital signs (SBP, DBP, PR, RR, body temperature).
Physical examination findings.
ECG results.
Pregnancy test (female subjects of child-bearing potential).

Criteria for Evaluation of Efficacy

Primary Efficacy Endpoints:
Time to use of conventional attack treatment.

Secondary Efficacy Endpoints:
Proportion of HAE attacks that progress by one level or more on the 5LS or that require conventional attack treatment within 12 h of study drug.
Time between treatment and (1) progression of global attack severity on the 5LS by one level or more, or (2) use of conventional attack treatment, whichever comes first within 12 h.

Exploratory Endpoints:
Cumulative global attack severity on the 5LS following study drug expressed as area under the curve (AUC) for the compound 600 mg vs. placebo.
Proportion of HAE attacks that require conventional attack treatment.
Proportion of HAE attacks that are rated “worse” or “much worse” on the 7TQ.
Proportion of HAE attacks that are rated “better” or “much better” on the 7TQ.
Time from study drug administration to complete HAE attack resolution (rating of none) on global attack severity scale (5LS).
Time to HAE attack being rated worse or much worse on the 7TQ.
Time to HAE attack being rated better or much better on the 7TQ.

General Statistical Methods and Types of Analyses

Analysis Sets:

Safety set (SAF): Subjects who have taken at least one dose of study drug (including the study drug dose in Part 1).
Full analysis set (for efficacy) (FAS): All randomized subjects who received both doses of study drug in Part 2.
Per protocol set (for efficacy) (PPS): Randomized subjects in Part 2 who received both doses of study drug (the compound) in Part 2 and have no major protocol deviations.
PK/PD analysis set: All subjects for whom PK/PD samples were taken in Part 1.

Sample Size:

A sample size of 50 subjects (25 per sequence) was proposed to provide 90% power for testing at the 5% alpha level (2-sided) for the primary endpoint of time to use of conventional attack treatment. This sample size was derived based upon an assumption that 40% of subjects will use conventional attack treatment while on the control arm while 10% will use conventional attack treatment on the experimental arm and that within subject data has minimal correlation. The assumption of minimal correlation should be a conservative assumption with respect to sample size. 68 subjects were enrolled to ensure that 50 subjects complete the study. 53 subjects completed part 2.

An oversampling by 20% (10 subjects) was proposed to account for subjects that may not complete both treatment periods due to infrequent or ineligible HAE attacks or for subjects who discontinue the trial early, for whatever reason. Thus, study enrolment was considered sufficient to address the primary efficacy hypothesis after 50 subjects have completed both treatment periods. Since further exposure was not required and could be considered unnecessary, ongoing subjects who have not completed both periods were asked to return to the study site and complete Visit 4 (Early Discontinuation visit). Data from all subjects, complete and incomplete, was analyzed in the safety set.

General Considerations:

Individual subject data was presented in subject data listings. Appropriate descriptive statistics was calculated for continuous and categorical data and summarized in tabular format.

Sample Analyses:

AEs were coded using the Medical Dictionary for Regulatory Activities (MedDRA) dictionary (v21.0 or higher) and classified by preferred term and system organ class (SOC). Listings of treatment-emergent adverse events (TEAEs), serious TEAEs, and TEAEs causing premature discontinuation will be provided by sequence group, and further classified by TEAE severity and relationship to study drug.

Efficacy Analyses:

Primary Endpoint

The primary endpoint, time to use of conventional attack treatment, was analyzed using a generalization of Gehan's test proposed by Feingold and Gillespie (1996) (Crossover trials with censored data. Statistics in Medicine 1996; 15(10): 953-967) to reflect the repeat measures on each subject. Subjects were treated as censored if no worsening occurs within 12 h of study drug.

Secondary Endpoints

The proportion of HAE attacks that worsen by one level or more on the 5LS or that require conventional attack treatment within 12 h of study drug was analyzed using Prescott's test (1981) (The comparison of success rates in cross-over trials in the presence of an order effect. Applied Statistics 1981; 30: 9-15) to compare the treatment arms.

A similar approach to that used for the primary endpoint was followed for the analysis of the time between study drug and HAE attack worsening by one level or more on the 5LS or use of conventional attack treatment, whichever comes first within 12 h. In addition to the tests described above, descriptive statistics were presented for the primary, secondary and exploratory endpoints, in each case comparing the compound to placebo, such as:
Cumulative global attack severity on the 5LS following study drug expressed as AUC for the compound 600 mg vs. placebo.
Proportion of HAE attacks that required conventional attack treatment.
Proportion of HAE attacks that were rated “worse” or “much worse” on the 7TQ.
Proportion of HAE attacks that were rated “better” or “much better” on the 7TQ.
Time from study drug administration to complete HAE attack resolution (rating of none) on global attack severity scale (5LS).
Time to HAE attack being rated worse or much worse on the 7TQ.
Time to HAE attack being rated better or much better on the 7TQ.

PK Analysis:

Non-compartmental PK parameters included maximum concentration in plasma (Cmax), time to reach Cmax in plasma (tmax), and area under the curve from time 0 to last sample (AUC0-t). Compartmental PK modelling described the PK of the compound and generate underlying Cmax, tmax, AUC, apparent clearance (CL/F), apparent volume of distribution (Vd/F) and estimated terminal elimination half-life (t %).

The PK parameters of the compound were determined from the individual concentration versus time data using Phoenix WinNonlin. In case of a deviation from the theoretical time, the actual time of blood sample was used in the calculation of the derived PK parameters. Individual concentrations and derived PK parameters of the compound in plasma were listed and summarized for each treatment. Individual and geometric mean concentration-time data were plotted on linear and semi-logarithmic scales.

PD Analysis:

The compound's effect on plasma kallikrein (PKa) activity was analyzed using two exploratory measures of PKa enzyme activity in plasma:
An assay to determine inhibition of exogenously activated plasma kallikrein enzyme activity from plasma samples obtained before and after receiving the compound.
An assay to measure the level of protection of cleavage of high molecular weight kininogen (HK) substrate (contained in whole plasma) from plasma kallikrein enzyme activity.

The PD were summarized for each treatment. Individual and mean data were provided as a report addendum located in the appendix of the final Clinical Study Report.

Preliminary PK data from Part 1 of the study:

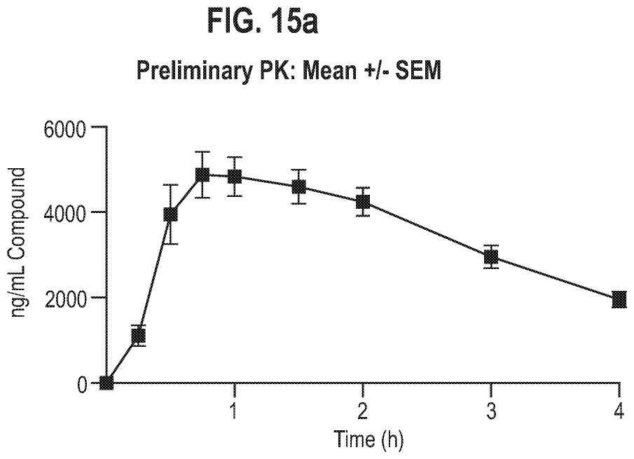

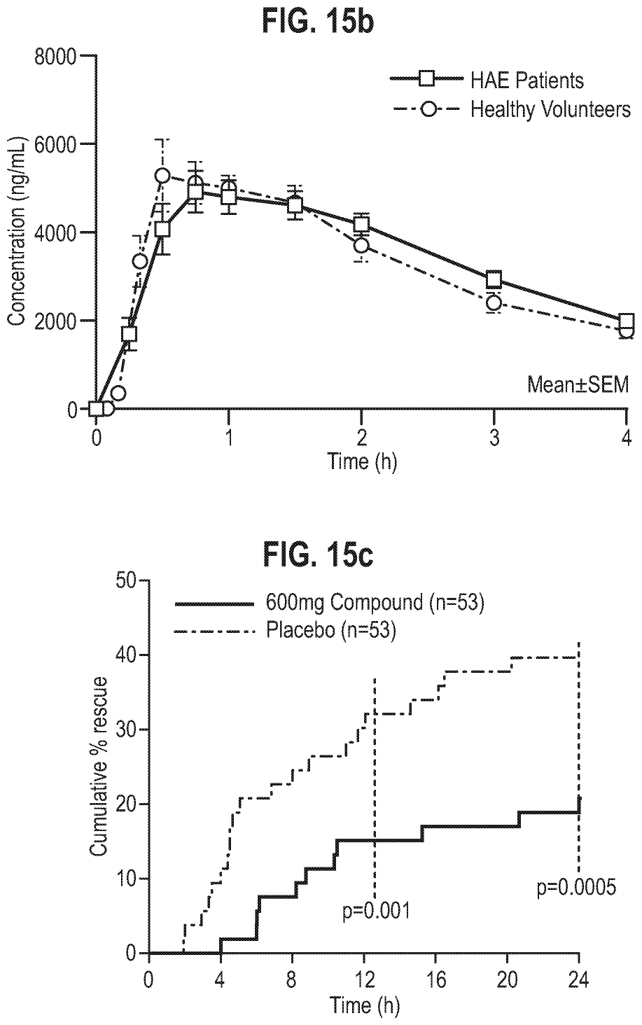

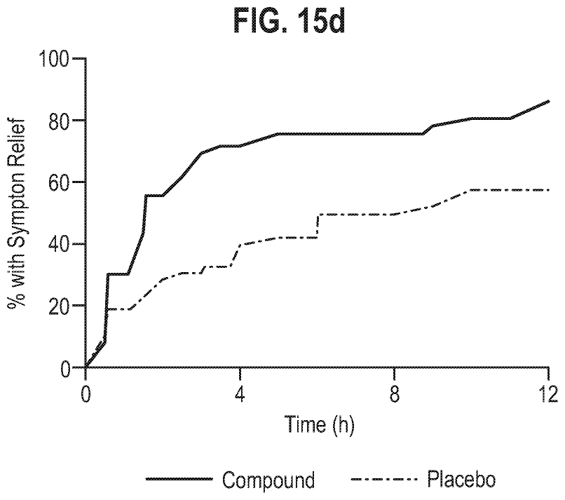

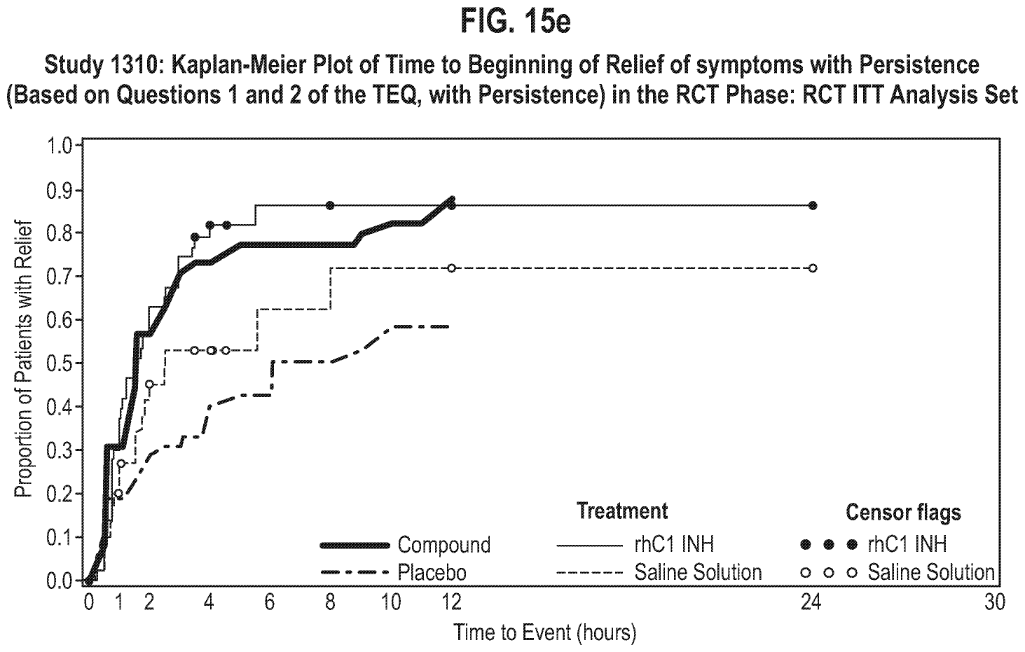

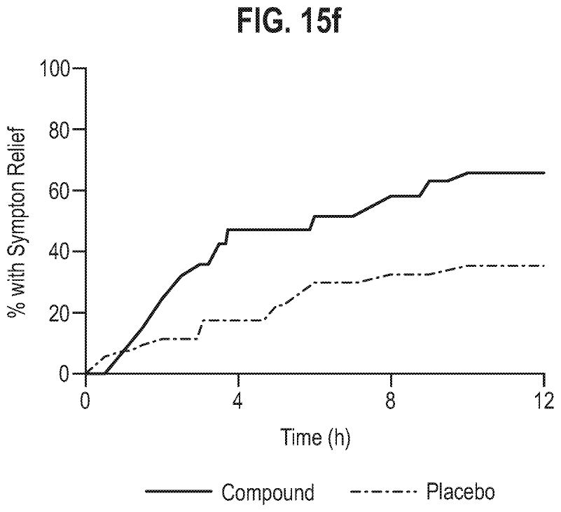

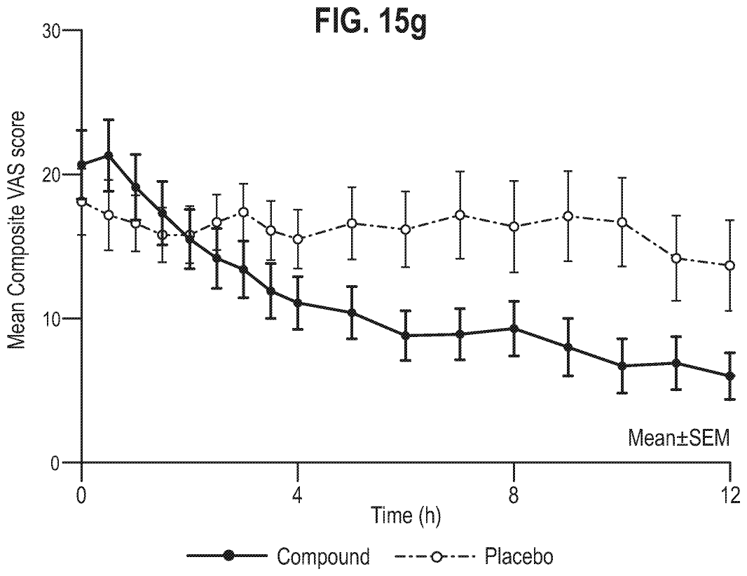

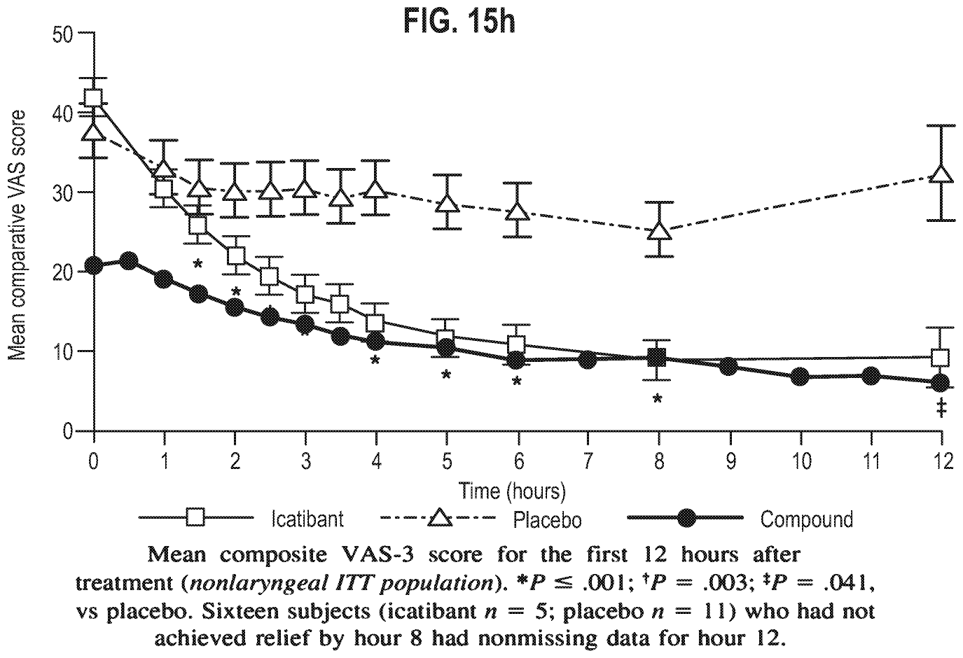

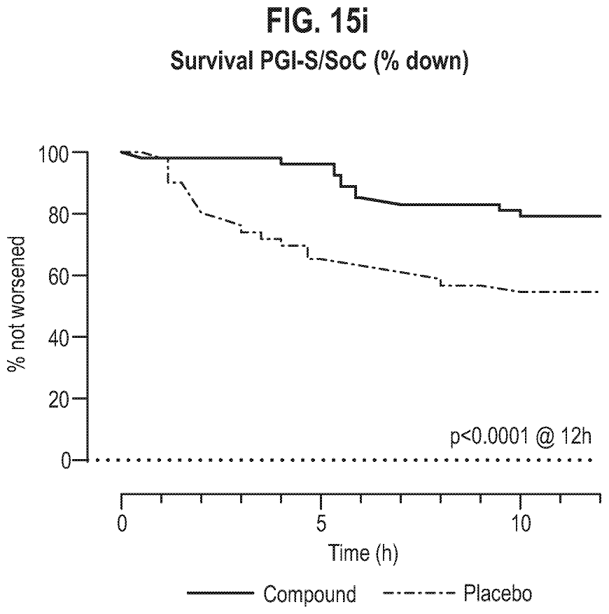

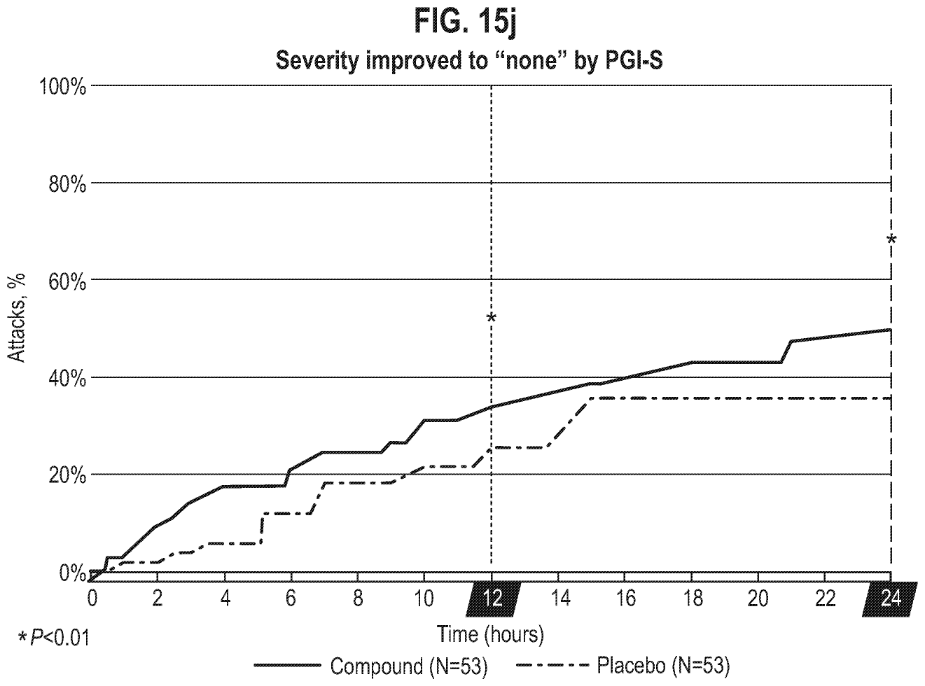

Preliminary PK data from 27 HAE patients was collated and analysed, and are shown in Table 4 and FIG. 15a.

TABLE 4

| | Cmax (ng/mL) |
|---|---|
| | Mean (95% CI) |
| Dose | n = 27 |
| 600 mg | 5907 (4913, 6901) |

Thus, these preliminary results show that the compound of Formula A demonstrates a pharmacokinetic profile that is suitable for on-demand oral administration in HAE patients.

FIG. 15b shows the complete PK profile of HAE patients from part 1, overlaid with PK data from healthy volunteers.

Efficacy results from Part 2 of the study

Primary Endpoint

Attacks treated with the compound significantly reduced use of rescue (p=0.0010) with 15.1% of compound treated attacks rescued compared to 30.2% on placebo at 12 hours. This efficacy benefit of the compound was maintained at 24 hours (p=0.0005). At 24 hours, use of rescue medication (p=0.0005) was 20.8% for the compound versus 39.6% on placebo.

FIG. 15c shows the cumulative % of patients that used rescue medication within 12 hours of being administered the compound.

Secondary Endpoints

The compound significantly reduced time to onset of symptom relief (p=<0.0001) on a Patient Global Impression of Change scale (PGI-C) (also known as the 7TQ) with a median time of 1.6 hours versus 9 hours for attacks treated with placebo. This demonstrates that treatment with the compound achieved symptom relief more quickly than placebo.

Within 12 hours of study drug, 83.0% of attacks treated with the compound were rated “a little better” or higher (when assessed according to the 7TQ), compared to 50.9% of attacks treated with Placebo (p<0.005). Within 24 hours of study drug, 84.9% of attacks treated with the compound were rated “a little better” or higher (when assessed according to the 7TQ), compared to 64.2% of attacks treated with Placebo (p<0.05).

Within 12 hours of study drug, 58.7% of attacks treated with the compound were rated “better” or higher (when assessed according to the 7TQ), compared to 35.8% of attacks treated with placebo (p=0.0319). Within 24 hours of study drug, 67.9% of attacks treated with the compound were rated “better” or higher (when assessed according to the 7TQ), compared to 47.2% of attacks treated with placebo (p=0.0593).

Treatment with the compound led to a statistically significant shortening of the median time for attacks to be rated “better” or higher (when assessed according to the 7TQ), with a median time of 5 hours for treatment with the compound compared to a median time of 15 hours for placebo (p=0.0036).

FIG. 15d shows the cumulative % of patients with symptom relief (as assessed using the 7TQ method) within 12 hours of being administered the compound. FIG. 15e compares time to symptom relief data for the compound with known clinical data for approved injectable product Ruconest® (see Charles M. Maplethorpe, MD, PhD. Clinical Reviewer. Summary Basis of Approval, Recombinant C1 Esterase Inhibitor, STN: 125495/0).

Attacks treated with the compound achieved symptom relief more quickly than placebo treated attacks (p<0.0001) when assessed using a composite Visual Analogue Scale (VAS) score (abdominal pain, skin pain, and skin swelling) with a median time to symptom relief of 6 hours versus >12 hours for placebo. Again, this demonstrated that treatment with the compound achieved symptom relief more quickly than placebo.

FIG. 15f shows the cumulative % of patients with symptom relief (as assessed using the composite VAS score) within 12 hours of being administered the compound.

Attacks treated with the compound reduced the severity of the HAE attack (as assessed using the composite VAS score) over 12 hours (p=0.0008) and 24 hours (p=0.0005). Within 12 hours of study drug, 79.2% of attacks treated with the compound do not worsen, compared to 54.7% of attacks treated with Placebo.

FIG. 15g shows the mean composite VAS score within 12 hours of being administered the compound. FIG. 15g is time- and baseline-adjusted AUC and excludes assessments post-rescue medication. FIG. 15h compares the mean composite VAS score for patients treated with the compound with known clinical data for approved injectable product Firazyr® (active ingredient, icatibant) (see Lumry et al., Ann Allergy Asthma Immunol. 2011; 107:529-537).

Within 12 hours of oral administration, the compound significantly increased the number of attacks stabilized or improved when assessed by a Patient Global Impression of Severity scale (PGI-S) (also known as the 5LS) or use of rescue (p<0.0001).

FIG. 15i shows the % of HAE attacks that did not worsen (as assessed using the 5LS method) within 12 hours of being administered the compound. As noted above, within 12 hours of study drug, 79.2% of attacks treated with the compound do not worsen, compared to 54.7% of attacks treated with Placebo. Within 24 hours of study drug, 69.8% of attacks treated with the compound do not worsen, compared to 50.9% of attacks treated with Placebo.

FIG. 15j shows the % of HAE attacks where severity improved to “none” when assessed using the PGI-S (also called the 5LS method). Within 12 hours of study drug, 56.6% of attacks treated with the compound improved to “none”, compared to 30.2% of attacks treated with Placebo (p<0.05). Within 24 hours of study drug, 66.0% of attacks treated with the compound improved to “none”, compared to 37.7% of attacks treated with Placebo (p<0.01).

Safety

There were no serious adverse events reported in the trial and no patients withdrew due to adverse events. In the open-label phase (Part 1), 8 on-treatment drug-related treatment emergent adverse events (TEAE) were experienced by 5 patients. In the crossover phase of the trial (Part 2), 3 on-treatment drug-related TEAEs were experienced by 3 patients (5.2%) following administration of the compound, and 2 on-treatment drug-related TEAEs were experienced by 2 patients (3.6%) following administration of placebo. These data are shown below.

| | Part 1 | Part 2 | Part 2 |
|---|---|---|---|
| | KVD900 | KVD900 | Placebo |
| | N = 68 | N = 58 | N = 55 |
| Total (events/patients) | 8/5 (7.4%) | 3/3 (5.2%) | 2/2 (3.6%) |
| Gastrointestinal Disorders | 1/1 (1.5%) | 1/1 (1.7%) | 1/1 (1.8%) |
| Abdominal Pain Upper | — | 1/1 (1.7%) | — |
| Anal Incontinence | — | — | 1/1 (1.8%) |
| Nausea | 1/1 (1.5%) | — | — |
| General Disorders | 1/1 (1.5%) | — | — |
| Malaise | 1/1 (1.5%) | — | — |
| Musculoskeletal Disorders | 1/1 (1.5%) | 1/1 (1.7%) | — |
| Back Pain | 1/1 (1.5%) | 1/1 (1.7%) | — |
| Nervous System Disorders | 3/3 (4.4%) | 1/1 (1.7%) | 1/1 (1.8%) |
| Dizziness | 1/1 (1.5%) | — | — |
| Headache | 2/2 (2.9%) | 1/1 (1.7%) | 1/1 (1.8%) |
| Vascular Disorders | 2/2 (2.9%) | — | — |
| Flushing | 2/2 (2.9%) | — | — |

Example 7—Phase 1 multiple dose study in healthy adult subjects

Aim: To evaluate the safety, tolerability, pharmacokinetics, and the change from baseline in QTc following administration of the compound formulated as 100 mg film coated tablets in healthy adult subjects.

Primary Objective:
To investigate the safety and tolerability of multiple doses of the compound.

Secondary Objectives:
To investigate the pharmacokinetics (PK) of multiple doses of the compound.
To evaluate the effects of the compound on ECG parameters, including concentration-QTc relationship, following administration of the compound 100 mg Film Coated Tablets (KalVista Pharmaceuticals) to healthy adult subjects.

Exploratory Objectives:
To investigate the pharmacodynamics (PD) of multiple doses of the compound.

Methods:

This is a phase 1, double-blind, placebo-controlled, multiple-dose, multiple-cohort study to evaluate safety and tolerability of the compound as well as of the ECG effects of the compound formulated as 100 mg Film Coated Tablets in healthy adult male and female subjects.

Four (4) cohorts are planned for evaluation. Cohorts 1, 2 and 3 will include 8 subjects each. Cohort 4 will include 18 subjects. Every attempt will be made to include an equal number of male and female subjects in each cohort.

During the study, oral doses of 600 mg of the compound as Film Coated Tablets (six 100 mg tablets) or 6 matching placebo tablets will be administered once every 8 hours (Cohort 1) every 4 hours (Cohort 2), or every 2 hours (Cohort 3 and 4) to healthy adult male and female subjects up to a total dose of 1800 mg. In Cohorts 1, 2 and 3, 6 subjects will receive the compound as 100 mg Film Coated Tablets and 2 subjects will receive the placebo for a total of 8 subjects per cohort. In Cohort 4, 12 subjects will receive the compound as 100 mg Film Coated Tablets and 6 subjects will receive the placebo for a total of 18 subjects.

Progression from Cohort 1 to Cohort 2 and Cohort 2 to Cohort 3 will occur after review of the safety data (labs, vital signs, safety ECGs, and adverse events) captured during the conduct of Cohort 1 and Cohort 2. Progression to Cohort 4 will occur after review of the safety data and pharmacokinetic data from Cohort 3. The pharmacokinetic data from Cohort 3 will be reviewed to ensure that the Cmax of the 3rd dose is high enough to support the evaluation of the change in the QTc interval from baseline.

A Holter monitor will be attached to each subject in order to continuously record ECGs. The monitor will be attached 1 hour before the first dose and will remain attached until after the final blood sample collection. The electrodes for the Holter monitor will be checked by a member of the clinic staff at appropriate intervals to ensure they are attached.

Blood samples will be collected at pre-dose, at intervals after the first dose, and at intervals over 24 hours after the final (third) dose (40 hours from the initial dose in Cohort 1, 32 hours from the initial dose in Cohort 2, 28 hours from the initial dose in Cohorts 3 and 4) in each cohort. Subjects will be confined to the clinical facility from at least 10 hours before dosing until after the final blood sample collection in each study cohort and will return to the clinic 5 to 7 days after the final dose for safety evaluations.

The pharmacokinetics of the compound will be measured by a fully validated analytical procedure and the pharmacodynamic effect on plasma kallikrein inhibition enzyme activity will be evaluated by an exploratory pharmacodynamic assessment.

Statistical analysis will be performed to evaluate the relationship between plasma drug concentrations and the change from baseline in ECG effects of the test formulation.

Treatment Administration

Cohort 1

The subjects will receive the test or placebo treatment every 8 hours over a 16-hour period (3 administrations of: 6×100 mg of the compound as 100 mg Film Coated Tablets or placebo dose administrations at 0, 8, and 16 hours, total dose of 1800 mg of the compound or placebo) according to a two-treatment randomization schedule under direct observation. Each dose will be administered with 240 mL of room temperature water. Subjects will be instructed to swallow the tablets whole without chewing or biting. Any subject who bites or chews the tablets will be dropped from the study. Immediately after dosing a mouth check will be performed

Cohort 2

The subjects will receive the test or placebo treatment every 4 hours over an 8-hour period (3 administrations of: 6×100 mg of the compound as 100 mg Film Coated Tablets or placebo dose administrations at 0, 4, and 8 hours, total dose of 1800 mg of the compound or placebo) according to a two-treatment randomization schedule under direct observation. Each dose will be administered with 240 mL of room temperature water. Subjects will be instructed to swallow the tablets whole without chewing or biting. Any subject who bites or chews the tablets will be dropped from the study. Immediately after dosing a mouth check will be performed to ensure that the tablets were swallowed whole without chewing or biting.

Cohort 3 and 4

The subjects will receive the test or placebo treatment every 2 hours over a 4-hour period (3 administrations of: 6×100 mg of the compound as 100 mg Film Coated Tablets or placebo dose administrations at 0, 2, and 4 hours, total dose of 1800 mg of the compound or placebo) according to a two-treatment randomization schedule under direct observation. Each dose will be administered with 240 mL of room temperature water. Subjects will be instructed to swallow the tablets whole without chewing or biting. Any subject who bites or chews the tablets will be dropped from the study. Immediately after dosing a mouth check will be performed to ensure that the tablets were swallowed whole without chewing or biting.

All subjects will fast (except water) for at least 8 hours before the first dosing. After initial dosing, subjects will continue to fast until at least 6 hours after the first dose.

Method of Assigning Subjects to Treatment Groups:

Cohort 1, 2 and 3

Subjects will be randomized such that 6 subjects will receive the test product and 2 subjects will receive the placebo. As a safety measure, a sentinel dosing scheme will be incorporated for each cohort, in which one subject will receive the test product and one subject will receive the placebo product followed by the remainder of the cohort.

Cohort 4

Subjects will be randomized such that 12 subjects receive the test product and 6 subjects receive the placebo.

The randomization schedule will be generated prior to the first dosing cohort using SAS®, Version 9.4 or higher.

Results:

No serious adverse events were reported during the study and no subjects were discontinued because of an AE. All reported adverse events were considered “mild” in severity and had an outcome of “recovered/resolved” at the end of the study.

No clinically relevant effects on the studied ECG parameters were identified.

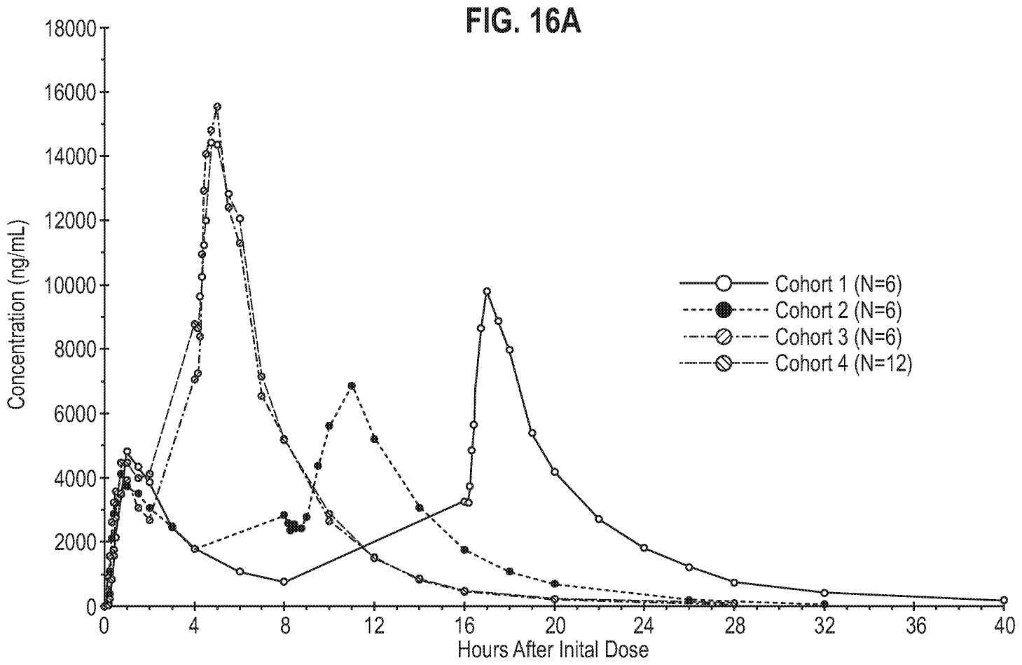

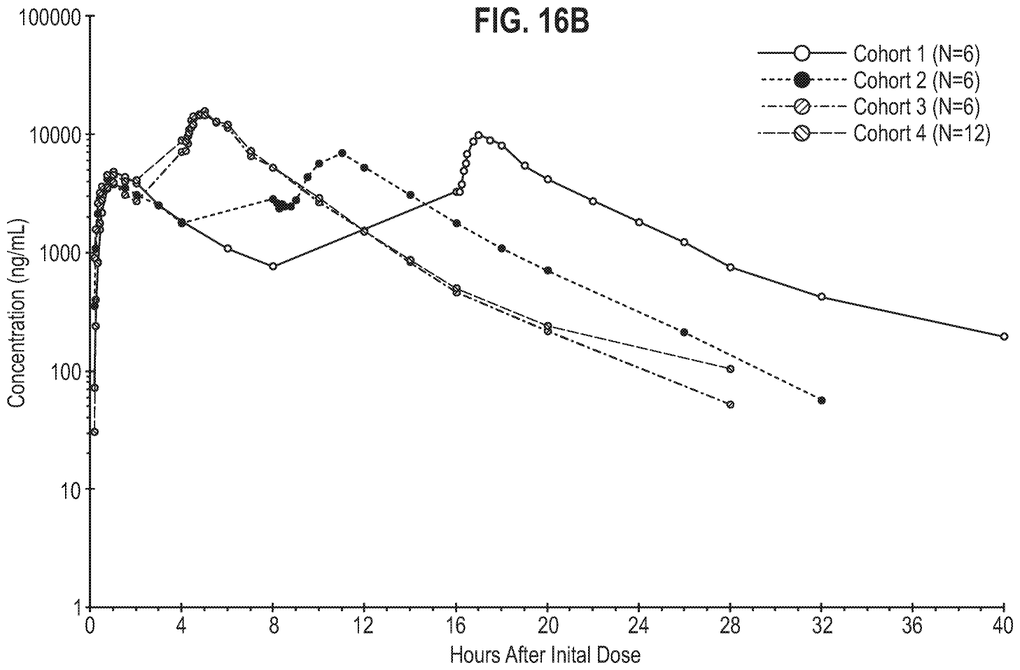

FIG. 16A shows the mean plasma concentrations of the compound of Formula A after the initial dose for each cohort.

FIG. 16B shows the mean plasma concentrations (semi-logarithmic scale) of the compound for formula A for each cohort.

These data demonstrate that the compound of Formula A has a pharmacokinetic profile suitable for oral administration when administered in multiple dosage amounts. The results further suggest that the compound of Formula A can be dosed safely at regular intervals.

Example 8—preparation of oral dosage forms

Orodispersible Tablets

Composition for 40,000×25 mg orodispersible tablets (4 kg)

| Ingredient | Mass (g) | wt % |
|---|---|---|
| Compound of Formula A | 1,000 | 25 |
| Pearlitol Flash (co-processed | 2,956 | 73.9 |
| Mannitol/Maize starch) | | |
| Sucralose | 4 | 0.1 |
| Sodium stearyl fumarate | 40 | 1 |
| Total | 4,000 | 100 |

The ingredients other than sodium stearyl fumarate are first forced through an 850 μm mesh before being intimately mixed by blending. Next, the sodium stearyl fumarate (pre-screened through same mesh) is added and briefly tumbled through into the powder mixture. The blend is then directly compressed into tablets.

Chewable Tablets

Composition for 12,000×100 mg chewable tablets (4 kg)

| Component | Mass (g) | wt % |
|---|---|---|
| Compound of Formula A | 1,200 | 30 |
| Mannitol | 1,832 | 45.8 |
| Microcrystalline | 600 | 15 |
| cellulose | | |
| Hydroxypropyl cellulose | 200 | 5 |
| Croscarmellose sodium | 120 | 3 |
| Strawberry Flavour | 4 | 0.1 |
| Sucralose | 4 | 0.1 |
| Magnesium stearate | 40 | 1 |
| Total | 4,000 | 100 |

The ingredients other than magnesium stearate are first forced through an 850 μm mesh before being intimately mixed by blending. Next, the magnesium stearate is added and briefly tumbled through into the powder mixture. The blend is then directly compressed into tablets.

Minitab Formulation

Composition for 223,600×10 mg mini-tablets (4 kg of tablet cores)

| Ingredient | Mass (g) | wt % |
|---|---|---|
| Compound of Formula A | 2,236 | 55.9 |
| Microcrystalline cellulose | 1,396 | 34.9 |
| Croscarmellose sodium | 188 | 4.7 |
| Povidone | 140 | 3.5 |
| Magnesium stearate | 40 | 1 |
| Total (before coating) | 4,000 | 100 |
| PVA/Titanium oxide, PEG, Talc based coating | 160 | |

The ingredients other than magnesium stearate (and the coating) are first forced through an 850 μm mesh before being intimately mixed by blending. Next, half of the magnesium stearate is added and briefly tumbled through into the powder mixture. The blend is then granulated by roller compaction followed by milling before the remainder of the magnesium stearate is added by further blending. The lubricated granules are then compressed into min tabs before being film-coated with a PVA based coating formulation until a further 4% weight gain had been achieved.

Coated granules

Composition for 4 kg of granules before coating

| Ingredient | Mass (g) | wt % |
|---|---|---|
| Compound of Formula A | 2,236 | 55.9 |
| Microcrystalline cellulose | 1,416 | 35.4 |
| Croscarmellose sodium | 188 | 4.7 |
| Povidone | 140 | 3.5 |
| Magnesium stearate | 20 | 0.5 |
| Total (before coating) | 4,000 | 100 |
| PVA/Titanium oxide, PEG, Talc based coating | 160 | |

The ingredients other than magnesium stearate are first forced through an 850 m mesh before being intimately mixed by blending. Next, the magnesium stearate is added and briefly tumbled through into the powder mixture. The blend is then granulated by roller compaction following by milling. The granules are then film-coated in a fluid-bed processor with a PVA based coating formulation until a further 4% weight gain had been achieved.

Alternative Orodispersible Tablet

Composition for 40,000×25 mg orodispersible tablets (4 kg before drying)

| Ingredient | Mass (g) | wt % |
|---|---|---|
| Compound of Formula A | 1,000 | 25 |
| Microcrystalline cellulose | 1,508 | 37.7 |
| Sodium starch glycolate | 480 | 12 |
| Croscarmellose sodium | 480 | 12 |
| Camphor | 400 | 10 |
| Sodium saccharin | 12 | 0.3 |
| Magnesium stearate | 120 | 3 |
| Total | 4,000 | 100 |

The ingredients other than magnesium stearate are first forced through an 850 μm mesh before being intimately mixed by blending. Next, the magnesium stearate is added and briefly tumbled through into the powder mixture. The blend is then directly compressed into tablets before being held in a vacuum dryer at 60° C. for 12 hours. This sublimates the camphor leaving a porous structure within the tablet.

Oral Suspension

Composition for 2,000×200 mg doses of oral suspension (10 kg)

| Ingredient | Mass (g) | wt % |
|---|---|---|
| Compound of Formula A | 400 | 4 |
| Sorbitol solution (70%) | 7,000 | 70 |
| Glycerol | 500 | 5 |
| Dispersible cellulose | 100 | 1 |
| Sodium carboxymethylcellulose | 300 | 3 |
| Polysorbate 80 | 50 | 0.5 |
| Methyl hydroxybenzoate | 2 | 0.02 |
| Sodium benzoate | 2 | 0.02 |
| Sodium saccharin | 10 | 0.1 |
| Ethanol | 200 | 2 |
| Vanilla Flavour | 10 | 0.1 |
| Strawberry Flavour | 10 | 0.1 |
| Purified water | q.s. to | |
| | 10,000 | |
| Total | 10,000 | 100 |

The dispersible cellulose, sodium carboxymethylcellulose and polysorbate are dissolved in approximately 800 mL of purified water before the sorbitol liquid, glycerol, ethanol, preservatives, sweetener and flavourings are added under constant stirring. The active ingredient, pre-screened through a 100 μm mesh, is added under constant stirring. Once homogenous, the suspension is diluted with purified water until the target weight is achieved. The suspension is then bottled and capped.

Numbered Embodiments
1. A method for treating bradykinin mediated angioedema on-demand comprising: orally administering the compound of Formula A (or a pharmaceutically acceptable salt and/or solvate thereof) to a patient in need thereof on-demand, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet, wherein the compound of Formula A is,

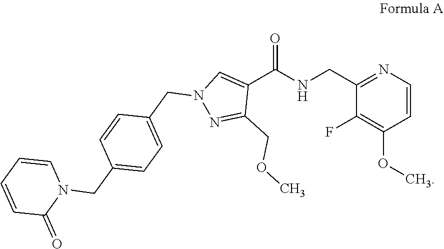 [SMILES: COCc1nn(Cc2ccc(Cn3ccccc3=O)cc2)cc1C(=O)NCc1nccc(OC)c1F]

2. The compound of Formula A (or a pharmaceutically acceptable salt and/or solvate thereof) for use in treating bradykinin mediated angioedema on-demand comprising: orally administering the compound of Formula A (or a pharmaceutically acceptable salt and/or solvate thereof) to a patient in need thereof on-demand, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet, wherein the compound of Formula A is,

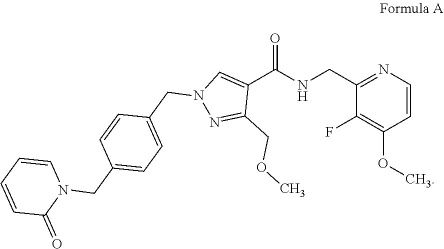 [SMILES: COCc1nn(Cc2ccc(Cn3ccccc3=O)cc2)cc1C(=O)NCc1nccc(OC)c1F]

3. The use of the compound of Formula A (or a pharmaceutically acceptable salt and/or solvate thereof) in the manufacture of a medicament for the treatment of bradykinin mediated angioedema on-demand comprising: orally administering the compound of Formula A (or a pharmaceutically acceptable salt and/or solvate thereof) to a patient in need thereof on-demand, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet, wherein the compound of Formula A is,

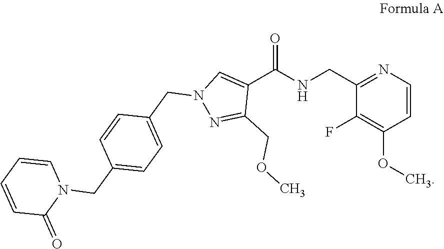 [SMILES: COCc1nn(Cc2ccc(Cn3ccccc3=O)cc2)cc1C(=O)NCc1nccc(OC)c1F]

4. The method according to numbered embodiment 1; the compound of Formula A (or a pharmaceutically acceptable salt and/or solvate thereof) for use according to numbered embodiment 2; or the use according to numbered embodiment 3;
   wherein the bradykinin mediated angioedema is hereditary angioedema (HAE); or wherein the bradykinin mediated angioedema is bradykinin-mediated angioedema non-hereditary (BK AEnH).
5. The method according to numbered embodiment 4; the compound of Formula A (or a pharmaceutically acceptable salt and/or solvate thereof) for use according to numbered embodiment 4; or the use according to numbered embodiment 4;
   wherein the bradykinin mediated angioedema is hereditary angioedema (HAE).
6. The method according to numbered embodiment 4; the compound of Formula A (or a pharmaceutically acceptable salt and/or solvate thereof) for use according to numbered embodiment 4; or the use according to numbered embodiment 4;
   wherein the bradykinin mediated angioedema is bradykinin-mediated angioedema non-hereditary (BK-AEnH).
7. The method according to numbered embodiment 6, the compound of Formula A (or a pharmaceutically acceptable salt and/or solvate thereof) for use according to numbered embodiment 6, or the use of a compound of Formula A (or a pharmaceutically acceptable salt and/or solvate thereof) according to numbered embodiment 6,
   wherein the bradykinin-mediated angioedema non-hereditary (BK-AEnH) is not caused by an inherited genetic dysfunction, fault, or mutation.
8. The method according to numbered embodiment 7, the compound of Formula A (or a pharmaceutically acceptable salt and/or solvate thereof) for use according to numbered embodiment 7, or the use of a compound of Formula A (or a pharmaceutically acceptable salt and/or solvate thereof) according to numbered embodiment 7,
   wherein the BK-AEnH is selected from: non-hereditary angioedema with normal C1 Inhibitor (AE-nC1 Inh), which can be environmental, hormonal, or drug-induced; acquired angioedema; anaphylaxis associated angioedema; angiotensin converting enzyme (ACE) inhibitor-induced angioedema; dipeptidyl peptidase-4 inhibitor-induced angioedema; and tPA-induced angioedema (tissue plasminogen activator-induced angioedema).
9. The method according to numbered embodiment 7, the compound of Formula A (or a pharmaceutically acceptable salt and/or solvate thereof) for use according to numbered embodiment 7, or the use of a compound of Formula A (or a pharmaceutically acceptable salt and/or solvate thereof) according to numbered embodiment 7,
   wherein, the AE-nC1 Inh is environmentally-induced by air pollution and/or silver nanoparticles.
10. The method according to any of numbered embodiments 1 or 4 to 9, the compound of Formula A (or a pharmaceutically acceptable salt and/or solvate thereof) for use according to any of numbered embodiments 2 or 4 to 9, or the use of the compound of Formula A (or a pharmaceutically acceptable salt and/or solvate thereof) according to any of numbered embodiments 3 or 4 to 9,
   wherein the patient is aged between 2 and less than 18.
11. The method according to numbered embodiment 10, the compound of Formula A (or a pharmaceutically acceptable salt and/or solvate thereof) for use according to numbered embodiment 10, or the use of the compound of Formula A (or a pharmaceutically acceptable salt and/or solvate thereof) according to numbered embodiment 10,
   wherein the patient is aged between 2 and less than 12.
12. The method according to any of numbered embodiments 1 or 4 to 9, the compound of Formula A (or a pharmaceutically acceptable salt and/or solvate thereof) for use according to any of numbered embodiments 2 or 4 to 9, or the use of the compound of Formula A (or a pharmaceutically acceptable salt and/or solvate thereof) according to any of numbered embodiments 3 or 4 to 9,
   wherein the patient is aged 70 years or older.
13. The method according to any of numbered embodiments 1 or 4 to 12, the compound of Formula A (or a pharmaceutically acceptable salt and/or solvate thereof) for use according to any of numbered embodiments 2 or 4 to 12, or the use of the compound of Formula A (or a pharmaceutically acceptable salt and/or solvate thereof) according to any of numbered embodiments 3 or 4 to 12,
   wherein the compound of Formula A (or a pharmaceutically acceptable salt and/or solvate thereof) is for use in treating an attack of bradykinin mediated angioedema on-demand and is orally administered on-demand upon recognition of a symptom of a bradykinin mediated angioedema attack.
14. The method according to numbered embodiment 13, the compound of Formula A (or a pharmaceutically acceptable salt and/or solvate thereof) for use according to numbered embodiment 13, or the use of the compound of Formula A (or a pharmaceutically acceptable salt and/or solvate thereof) according to numbered embodiment 13,
   wherein the patient is suffering from a laryngeal attack.
15. The method according to any of numbered embodiments 13 or 14, the compound of Formula A (or a pharmaceutically acceptable salt and/or solvate thereof) for use according to any of numbered embodiment 13 or 14, or the use of the compound of Formula A (or a pharmaceutically acceptable salt and/or solvate thereof) according to any of numbered embodiment 13 or 14,
   wherein the symptom of an acute HAE attack recognised is at least one of: swelling of tissues; fatigue; headache; muscle aches; skin tingling; abdominal pain; nausea; vomiting; diarrhoea; difficulty swallowing; hoarseness; shortness of breath; and/or mood changes.
16. The method according to any of numbered embodiments 13 to 15, the compound of Formula A (or a pharmaceutically acceptable salt and/or solvate thereof) for use according to any of numbered embodiments 13 to 15, or the use of the compound of Formula A (or a pharmaceutically acceptable salt and/or solvate thereof) according to any of numbered embodiments 13 to 15,
   wherein the compound of Formula A (or a pharmaceutically acceptable salt and/or solvate thereof) is orally administered on-demand within 1 hour of the symptom of a bradykinin mediated angioedema attack being recognised.
17. The method according to any of numbered embodiments 13 to 16, the compound of Formula A (or a pharmaceutically acceptable salt and/or solvate thereof) for use according to any of numbered embodiments 13 to 16, or the use of the compound of Formula A (or a pharmaceutically acceptable salt and/or solvate thereof) according to any of numbered embodiments 13 to 16,
   wherein the compound of Formula A (or a pharmaceutically acceptable salt and/or solvate thereof) is orally administered on-demand within 30 minutes, within 20 minutes, within 10 minutes, or within 5 minutes of the symptom of a bradykinin mediated angioedema attack being recognised.
18. The method according to any of numbered embodiments 13 to 17, the compound of Formula A (or a pharmaceutically acceptable salt and/or solvate thereof) for use according to any of numbered embodiments 13 to 17, or the use of the compound of Formula A (or a pharmaceutically acceptable salt and/or solvate thereof) according to any of numbered embodiments 13 to 17,
   wherein the compound of Formula A (or a pharmaceutically acceptable salt or solvate thereof) is orally administered on-demand in the prodromal phase of a bradykinin mediated angioedema attack.
19. The method according to numbered embodiment 18, the compound of Formula A (or a pharmaceutically acceptable salt and/or solvate thereof) for use according to numbered embodiment 18, or the use of the compound of Formula A (or a pharmaceutically acceptable salt and/or solvate thereof) according to numbered embodiment 18,
   wherein the symptom recognised is at least one of: a slight swelling, abdominal pain or reddening of the skin.
20. The method according to numbered embodiment 19, the compound of Formula A (or a pharmaceutically acceptable salt and/or solvate thereof) for use according to numbered embodiment 19, or the use of the compound of Formula A (or a pharmaceutically acceptable salt and/or solvate thereof) according to numbered embodiment 19,
   wherein the symptom recognised is erythema marginatum.
21. The method according to any of numbered embodiments 1 and 4 to 20, the compound of Formula A (or a pharmaceutically acceptable salt and/or solvate thereof) for use according to any of numbered embodiments 2 and 4 to 20, or the use of the compound of Formula A (or a pharmaceutically acceptable salt and/or solvate thereof) according to any of numbered embodiments 3 to 20,
   wherein the treatment shortens the duration of the bradykinin mediated angioedema attack.
22. The method according to numbered embodiment 18, the compound of Formula A (or a pharmaceutically acceptable salt and/or solvate thereof) for use according to numbered embodiment 18, or the use of the compound of Formula A (or a pharmaceutically acceptable salt and/or solvate thereof) according to numbered embodiment 18,
   wherein the treatment prevents the bradykinin mediated angioedema attack from progressing to the swelling stage of a bradykinin mediated angioedema attack.
23. The method according to any of numbered embodiments 1 or 4 to 12, the compound of Formula A (or a pharmaceutically acceptable salt and/or solvate thereof) for use according to any of numbered embodiments 2 or 4 to 12, or the use of the compound of Formula A (or a pharmaceutically acceptable salt and/or solvate thereof) according to any of numbered embodiments 3 or 4 to 12,
   wherein the compound of Formula A (or a pharmaceutically acceptable salt and/or solvate thereof) is orally administered on-demand to prophylactically reduce the likelihood of a bradykinin mediated angioedema attack.
24. The method according to numbered embodiment 23, the compound of Formula A (or a pharmaceutically acceptable salt and/or solvate thereof) for use according to numbered embodiment 23, or the use of the compound of Formula A (or a pharmaceutically acceptable salt and/or solvate thereof) according to numbered embodiment 23,
   wherein the compound of Formula A (or a pharmaceutically acceptable salt and/or solvate thereof) is orally administered on-demand when it is anticipated that a bradykinin mediated angioedema attack will be induced.
25. The method according to any of numbered embodiments 23 to 24, the compound of Formula A (or a pharmaceutically acceptable salt and/or solvate thereof) for use according to any of numbered embodiments 23 to 24, or the use of the compound of Formula A (or a pharmaceutically acceptable salt and/or solvate thereof) according to any of numbered embodiments 23 to 24,
   wherein the compound of Formula A (or a pharmaceutically acceptable salt and/or solvate thereof) is orally administered on-demand to prevent a bradykinin mediated angioedema attack.
26. The method according to any of numbered embodiments 23 to 25, the compound of Formula A (or a pharmaceutically acceptable salt and/or solvate thereof) for use according to any of numbered embodiments 23 to 25, or the use of a compound of Formula A (or a pharmaceutically acceptable salt and/or solvate thereof) according to any of numbered embodiments 23 to 25,
   wherein the BK-AEnH is tPA-induced angioedema,
   wherein the patient is also being administered a tissue plasminogen activator,
      wherein the compound of Formula A (or a pharmaceutically acceptable salt and/or solvate thereof) is administered prior to, during, or after administration of the tissue plasminogen activator to the patient.
27. The method according to any of numbered embodiments 24 to 25, the compound of Formula A (or a pharmaceutically acceptable salt and/or solvate thereof) for use according to any of numbered embodiments 24 to 25, or the use of the compound of Formula A (or a pharmaceutically acceptable salt and/or solvate thereof) according to any of numbered embodiments 24 to 25,
   wherein the compound of Formula A (or a pharmaceutically acceptable salt and/or solvate thereof) is orally administered on-demand when it is anticipated that a bradykinin mediated angioedema attack will be induced by physical traumata and/or stress.
28. The method according to numbered embodiment 27, the compound of Formula A (or a pharmaceutically acceptable salt and/or solvate thereof) for use according to numbered embodiment 27, or the use of the compound of Formula A (or a pharmaceutically acceptable salt and/or solvate thereof) according to numbered embodiment 27,
   wherein it is anticipated that a bradykinin mediated angioedema attack will be induced by the physical traumata of a dental procedure and/or the mental stress associated with a dental procedure.
29. A method for treating bradykinin mediated angioedema comprising: orally administering the compound of Formula A (or a pharmaceutically acceptable salt and/or solvate thereof) to a patient in need thereof, wherein the compound of Formula A (or a pharmaceutically acceptable salt and/or solvate thereof) is orally administered prophylactically to reduce the likelihood of a bradykinin mediated angioedema attack, wherein the compound of Formula A (or a pharmaceutically acceptable salt and/or solvate thereof) is administered regularly to the patient, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet, wherein the compound of Formula A is,

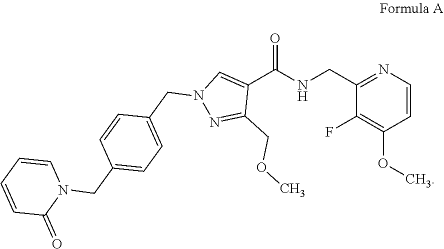 [SMILES: COCc1nn(Cc2ccc(Cn3ccccc3=O)cc2)cc1C(=O)NCc1nccc(OC)c1F]

30. The compound of Formula A (or a pharmaceutically acceptable salt and/or solvate thereof) for use in treating bradykinin mediated angioedema comprising: orally administering the compound of Formula A (or a pharmaceutically acceptable salt and/or solvate thereof) to a patient in need thereof, wherein the compound of Formula A (or a pharmaceutically acceptable salt and/or solvate thereof) is orally administered prophylactically to reduce the likelihood of a bradykinin mediated angioedema attack, wherein the compound of Formula A (or a pharmaceutically acceptable salt and/or solvate thereof) is administered regularly to the patient, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet, wherein the compound of Formula A is

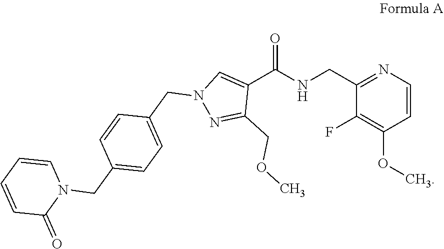 [SMILES: COCc1nn(Cc2ccc(Cn3ccccc3=O)cc2)cc1C(=O)NCc1nccc(OC)c1F]

31. The use of the compound of Formula A (or a pharmaceutically acceptable salt and/or solvate thereof) in the manufacture of a medicament for the treatment of bradykinin mediated angioedema comprising: orally administering the compound of Formula A (or a pharmaceutically acceptable salt and/or solvate thereof) to a patient in need thereof, wherein the compound of Formula A (or a pharmaceutically acceptable salt and/or solvate thereof) is orally administered prophylactically to reduce the likelihood of a bradykinin mediated angioedema attack, wherein the compound of Formula A (or a pharmaceutically acceptable salt and/or solvate thereof) is administered regularly to the patient, wherein the compound of Formula A (or a pharmaceutically acceptable salt and/or solvate thereof) is administered as an oral dosage form selected from a chewable tablet or soft gel, a mini-tablet, a powder, granules, a solution, an emulsion, a suspension, a syrup, a dispersible tablet, and an orodispersible tablet, wherein the compound of Formula A is

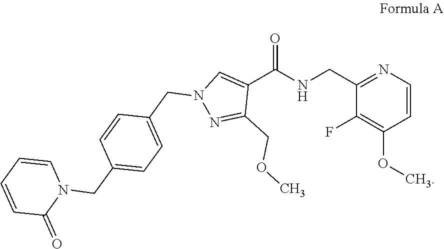 [SMILES: COCc1nn(Cc2ccc(Cn3ccccc3=O)cc2)cc1C(=O)NCc1nccc(OC)c1F]

32. The method according to numbered embodiment 29; the compound of Formula A (or a pharmaceutically acceptable salt and/or solvate thereof) for use according to numbered embodiment 30; or the use according to numbered embodiment 31;
   wherein the bradykinin mediated angioedema is hereditary angioedema (HAE); or wherein the bradykinin mediated angioedema is bradykinin-mediated angioedema non-hereditary (BK AEnH).
33. The method according to numbered embodiment 32; the compound of Formula A (or a pharmaceutically acceptable salt and/or solvate thereof) for use according to numbered embodiment 32; or the use according to numbered embodiment 32;
   wherein the bradykinin mediated angioedema is hereditary angioedema (HAE).
34. The method according to numbered embodiment 32; the compound of Formula A (or a pharmaceutically acceptable salt and/or solvate thereof) for use according to numbered embodiment 32; or the use according to numbered embodiment 32;
   wherein the bradykinin mediated angioedema is bradykinin-mediated angioedema non-hereditary (BK-AEnH).
35. The method according to numbered embodiment 34, the compound of Formula A (or a pharmaceutically acceptable salt and/or solvate thereof) for use according to numbered embodiment 34, or the use of a compound of Formula A (or a pharmaceutically acceptable salt and/or solvate thereof) according to numbered embodiment 34,
   wherein the bradykinin-mediated angioedema non-hereditary (BK-AEnH) is not caused by an inherited genetic dysfunction, fault, or mutation.
36. The method according to numbered embodiment 34, the compound of Formula A (or a pharmaceutically acceptable salt and/or solvate thereof) for use according to numbered embodiment 34, or the use of a compound of Formula A (or a pharmaceutically acceptable salt and/or solvate thereof) according to numbered embodiment 34,
   wherein the BK-AEnH is selected from: non-hereditary angioedema with normal C1 Inhibitor (AE-nC1 Inh), which can be environmental, hormonal, or drug-induced; acquired angioedema; anaphylaxis associated angioedema; angiotensin converting enzyme (ACE) inhibitor-induced angioedema; dipeptidyl peptidase-4 inhibitor-induced angioedema; and tPA-induced angioedema (tissue plasminogen activator-induced angioedema).
37. The method according to numbered embodiment 34, the compound of Formula A (or a pharmaceutically acceptable salt and/or solvate thereof) for use according to numbered embodiment 34, or the use of a compound of Formula A (or a pharmaceutically acceptable salt and/or solvate thereof) according to numbered embodiment 34,
   wherein, the AE-nC1 Inh is environmentally-induced by air pollution and/or silver nanoparticles.
38. The method according to any of numbered embodiments 29 or 32 to 37, the compound of Formula A (or a pharmaceutically acceptable salt and/or solvate thereof) for use according to any of numbered embodiment 30 or 32 to 37, or the use of a compound of Formula A (or a pharmaceutically acceptable salt and/or solvate thereof) according to any of numbered embodiment 31 or 32 to 37,
   wherein the compound of Formula A (or a pharmaceutically acceptable salt and/or solvate thereof) is administered to prevent a bradykinin mediated angioedema attack.
39. The method according to any of numbered embodiments 29 or 32 to 38, the compound of Formula A (or a pharmaceutically acceptable salt and/or solvate thereof) for use according to any of numbered embodiments 30 or 32 to 38, or the use of a compound of Formula A (or a pharmaceutically acceptable salt and/or solvate thereof) according to any of numbered embodiments 31 to 38,
   wherein the compound of Formula A (or a pharmaceutically acceptable salt and/or solvate thereof) is orally administered once daily.
40. The method according to any of numbered embodiments 29 or 32 to 38, the compound of Formula A (or a pharmaceutically acceptable salt and/or solvate thereof) for use according to any of numbered embodiments 30 or 32 to 38, or the use of a compound of Formula A (or a pharmaceutically acceptable salt and/or solvate thereof) according to any of numbered embodiments 31 to 38,
   wherein the compound of Formula A (or a pharmaceutically acceptable salt and/or solvate thereof) is orally administered twice daily.
41. The method according to any of numbered embodiments 29 or 32 to 38, the compound of Formula A (or a pharmaceutically acceptable salt and/or solvate thereof) for use according to any of numbered embodiments 30 or 32 to 38, or the use of a compound of Formula A (or a pharmaceutically acceptable salt and/or solvate thereof) according to any of numbered embodiments 31 to 38,
   wherein the compound of Formula A (or a pharmaceutically acceptable salt and/or solvate thereof) is orally administered three times daily.
42. The method according to any preceding numbered embodiment, the compound of Formula A (or a pharmaceutically acceptable salt and/or solvate thereof) for use according to any preceding numbered embodiment, or the use of a compound of Formula A (or a pharmaceutically acceptable salt and/or solvate thereof) according to any preceding numbered embodiment,
   wherein the compound (or a pharmaceutically acceptable salt and/or solvate thereof) (i) inhibits plasma kallikrein, (ii) reduces cleavage of plasma prekallikrein, and/or (iii) reduces the generation of Factor XIIa from Factor XII.
43. The method according to numbered embodiment 42, the compound of Formula A (or a pharmaceutically acceptable salt and/or solvate thereof) for use according to numbered embodiment 42, or the use of a compound of Formula A (or a pharmaceutically acceptable salt and/or solvate thereof) according to numbered embodiment 42,
   wherein the patient is administered a dose of the compound of Formula A (or a pharmaceutically acceptable salt and/or solvate thereof) such that the patient's plasma has a concentration of the compound of Formula A (or a pharmaceutically acceptable salt and/or solvate thereof) of at least 500 ng/mL.
44. The method according to numbered embodiment 43, the compound of Formula A (or a pharmaceutically acceptable salt and/or solvate thereof) for use according to numbered embodiment 43, or the use of a compound of Formula A (or a pharmaceutically acceptable salt and/or solvate thereof) according to numbered embodiment 43,
   wherein the patient is administered at least 60 mg of the compound of Formula A (or a pharmaceutically acceptable salt and/or solvate thereof).
45. The method according to any preceding numbered embodiment, the compound of Formula A (or a pharmaceutically acceptable salt and/or solvate thereof) for use according to any preceding numbered embodiment, or the use of a compound of Formula A (or a pharmaceutically acceptable salt and/or solvate thereof) according to any preceding numbered embodiment,
   wherein the compound of Formula A (or a pharmaceutically acceptable salt and/or solvate thereof) blocks contact system activation for up to six hours.
46. The method according to any preceding numbered embodiment, the compound of Formula A (or a pharmaceutically acceptable salt and/or solvate thereof) for use according to any preceding numbered embodiment, or the use of a compound of Formula A (or a pharmaceutically acceptable salt and/or solvate thereof) according to any preceding numbered embodiment,
   wherein the compound of Formula A (or a pharmaceutically acceptable salt and/or solvate thereof) is administered at a daily dosage amount of between 5 mg and 2000 mg.
47. The method according to any preceding numbered embodiment, the compound of Formula A (or a pharmaceutically acceptable salt and/or solvate thereof) for use according to any preceding numbered embodiment, or the use of a compound of Formula A (or a pharmaceutically acceptable salt and/or solvate thereof) according to any preceding numbered embodiment,
   wherein the compound of Formula A is administered at a daily dosage amount of between 100 mg and 1500 mg, between 300 mg to 1800 mg, between 100 mg and 1400 mg per day, between 200 mg and 1200 mg, between 300 mg and 1200 mg, between 600 mg and 1200 mg, between 450 mg and 900 mg, between 500 mg and 1000 mg, between 450 mg and 600 mg, between 500 mg and 700 mg, between 800 mg and 1000 mg per day, between 900 mg and 1400 mg, or between 900 mg and 1200 mg.
48. The method according to any preceding numbered embodiment, the compound of Formula A (or a pharmaceutically acceptable salt and/or solvate thereof) for use according to any preceding numbered embodiment, or the use of a compound of Formula A (or a pharmaceutically acceptable salt and/or solvate thereof) according to any preceding numbered embodiment,
   wherein the patient is administered the daily dosage amount in two dosage amounts within a 24 hour period starting from the time of taking the first dosage amount.
49. The method according to numbered embodiment 48, the compound of Formula A (or a pharmaceutically acceptable salt and/or solvate thereof) for use according to numbered embodiment 48, or the use of a compound of Formula A (or a pharmaceutically acceptable salt and/or solvate thereof) according to numbered embodiment 48,
   wherein the two dosage amounts are administered simultaneously, separately or sequentially.
50. The method according to any of numbered embodiments 48 or 49, the compound of Formula A (or a pharmaceutically acceptable salt and/or solvate thereof) for use according to any of numbered embodiments 48 or 49, or the use of a compound of Formula A (or a pharmaceutically acceptable salt and/or solvate thereof) according to any of numbered embodiments 48 or 49,
   wherein the second dosage amount is administered between 2 and 6 hours of the first, preferably between about 3 and 6 hours of the first dosage amount.
51. The method according to any of numbered embodiments 48 or 49, the compound of Formula A (or a pharmaceutically acceptable salt and/or solvate thereof) for use according to any of numbered embodiments 48 or 49, or the use of the compound of Formula A (or a pharmaceutically acceptable salt and/or solvate thereof) according to any of numbered embodiments 48 or 49,
   wherein the second dosage amount can be administered at least about 6 hours after the first dosage amount.
52. The method according to any of numbered embodiments 1, 4 to 29, or 32 to 47, the compound of Formula A (or a pharmaceutically acceptable salt and/or solvate thereof) for use according to any of numbered embodiments 2, 4 to 29, 30, or 32 to 47, or the use of a compound of Formula A (or a pharmaceutically acceptable salt and/or solvate thereof) according to any of numbered embodiments 3 to 29, or 31 to 47,
   wherein the patient is administered the compound of Formula A (or a pharmaceutically acceptable salt and/or solvate thereof) as three dosage amounts per day.
53. The method according to numbered embodiment 52, the compound of Formula A (or a pharmaceutically acceptable salt and/or solvate thereof) for use according to numbered embodiment 52, or the use of a compound of Formula A (or a pharmaceutically acceptable salt and/or solvate thereof) according to numbered embodiment 52,
   wherein the three dosage amounts are administered simultaneously, separately or sequentially.
54. The method according to any of numbered embodiments 52 or 53, the compound of Formula A (or a pharmaceutically acceptable salt and/or solvate thereof) for use according to any of numbered embodiments 52 or 53, or the use of the compound of Formula A (or a pharmaceutically acceptable salt and/or solvate thereof) according to any of numbered embodiments 52 or 53,
   wherein the second and third dosage amounts can be administered at least about 6 hours after the preceding dosage amount.
55. The method according to any of numbered embodiments 45 to 54, the compound of Formula A (or a pharmaceutically acceptable salt and/or solvate thereof) for use according to any of numbered embodiments 45 to 54, or the use of a compound of Formula A (or a pharmaceutically acceptable salt and/or solvate thereof) according to any of numbered embodiments 45 to 54,
   wherein each dosage amount comprises about 600 mg of the compound of formula A.
56. The method according to any preceding numbered embodiment, the compound of Formula A (or a pharmaceutically acceptable salt and/or solvate thereof) for use according to any preceding numbered embodiment, or the use of a compound of Formula A (or a pharmaceutically acceptable salt and/or solvate thereof) according to any preceding numbered embodiment,
   wherein the compound of Formula A (or a pharmaceutically acceptable salt and/or solvate thereof) is administered as a dispersible tablet or an orodispersible tablet.
57. The method according to numbered embodiment 56, the compound of Formula A (or a pharmaceutically acceptable salt and/or solvate thereof) for use according to numbered embodiment 56, or the use of a compound of Formula A (or a pharmaceutically acceptable salt and/or solvate thereof) according to numbered embodiment 56,
   wherein the compound of Formula A (or a pharmaceutically acceptable salt and/or solvate thereof) is administered as a dispersible tablet.
58. The method according to numbered embodiment 56, the compound of Formula A (or a pharmaceutically acceptable salt and/or solvate thereof) for use according to numbered embodiment 56, or the use of a compound of Formula A (or a pharmaceutically acceptable salt and/or solvate thereof) according to numbered embodiment 56,
   wherein the compound of Formula A (or a pharmaceutically acceptable salt and/or solvate thereof) is administered as an orodispersible tablet.
59. The method according to numbered embodiment 58, the compound of Formula A (or a pharmaceutically acceptable salt and/or solvate thereof) for use according to numbered embodiment 58, or the use of a compound of Formula A (or a pharmaceutically acceptable salt and/or solvate thereof) according to numbered embodiment 58,
   wherein the orodispersible dosage form comprises one or more of microcrystalline cellulose, sodium starch glycolate, croscarmellose sodium, camphor, sodium saccharin, and magnesium stearate.
60. The method according to numbered embodiment 58, the compound of Formula A (or a pharmaceutically acceptable salt and/or solvate thereof) for use according to numbered embodiment 58, or the use of a compound of Formula A (or a pharmaceutically acceptable salt and/or solvate thereof) according to numbered embodiment 58,
   wherein the orodispersible dosage form comprises one or more of pearlitol Flash (co-processed Mannitol/Maize starch), sucralose, and sodium stearyl fumarate.
61. The method according to numbered embodiment 58, the compound of Formula A (or a pharmaceutically acceptable salt and/or solvate thereof) for use according to numbered embodiment 58, or the use of a compound of Formula A (or a pharmaceutically acceptable salt and/or solvate thereof) according to numbered embodiment 58,
   wherein the orodispersible dosage form comprises polyvinyl polypyrolidone (e.g. Crospovidone).
62. The method according to any of numbered embodiments 1 to 62, the compound of Formula A (or a pharmaceutically acceptable salt and/or solvate thereof) for use according to any of numbered embodiments 1 to 62, or the use of a compound of Formula A (or a pharmaceutically acceptable salt and/or solvate thereof) according to any of numbered embodiments 1 to 62,
   wherein the compound of Formula A (or a pharmaceutically acceptable salt and/or solvate thereof) is administered as a mini-tablet.
63. The method according to numbered embodiment 62, the compound of Formula A (or a pharmaceutically acceptable salt and/or solvate thereof) for use according to numbered embodiment 62, or the use of a compound of Formula A (or a pharmaceutically acceptable salt and/or solvate thereof) according to numbered embodiment 62,
   wherein the mini-tablet comprises one or more of microcrystalline cellulose, croscarmellose sodium, polyvinylpyrrolidone (povidone), and magnesium stearate.
64. The method according to any of numbered embodiments 1 to 55, the compound of Formula A (or a pharmaceutically acceptable salt and/or solvate thereof) for use according to any of numbered embodiments 1 to 55, or the use of a compound of Formula A (or a pharmaceutically acceptable salt and/or solvate thereof) according to any of numbered embodiments 1 to 55,
   wherein the compound of Formula A (or a pharmaceutically acceptable salt and/or solvate thereof) is administered as a orodispersible film.
65. The method according to any of numbered embodiments 1 to 55, the compound of Formula A (or a pharmaceutically acceptable salt and/or solvate thereof) for use according to any of numbered embodiments 1 to 55, or the use of a compound of Formula A (or a pharmaceutically acceptable salt and/or solvate thereof) according to any of numbered embodiments 1 to 55,
   wherein the compound of Formula A (or a pharmaceutically acceptable salt and/or solvate thereof) is administered as a powder.
66. The method according to any of numbered embodiments 1 to 55, the compound of Formula A (or a pharmaceutically acceptable salt and/or solvate thereof) for use according to any of numbered embodiments 1 to 55, or the use of a compound of Formula A (or a pharmaceutically acceptable salt and/or solvate thereof) according to any of numbered embodiments 1 to 55,
   wherein the compound of Formula A (or a pharmaceutically acceptable salt and/or solvate thereof) is administered as granules.
67. The method according to numbered embodiment 66, the compound of Formula A (or a pharmaceutically acceptable salt and/or solvate thereof) for use according to numbered embodiment 66, or the use of a compound of Formula A (or a pharmaceutically acceptable salt and/or solvate thereof) according to numbered embodiment 66,
   wherein the granules comprises one or more of microcrystalline cellulose, croscarmellose sodium, polyvinylpyrrolidone (povidone), and magnesium stearate, optionally coated with one or more of PVA/Titanium oxide, PEG, or talc based coating.
68. The method according to any of numbered embodiments 56 to 67, the compound of Formula A (or a pharmaceutically acceptable salt and/or solvate thereof) for use according to any of numbered embodiments 56 to 67, or the use of a compound of Formula A (or a pharmaceutically acceptable salt and/or solvate thereof) according to any of numbered embodiments 56 to 67,
   wherein the disintegration specification can be >80% in less than about 10 minutes.
69. The method according to numbered embodiment 68, the compound of Formula A (or a pharmaceutically acceptable salt and/or solvate thereof) for use according to numbered embodiment 68, or the use of a compound of Formula A (or a pharmaceutically acceptable salt and/or solvate thereof) according to numbered embodiment 68,
   wherein the disintegration specification can be >80% in less than about 1 minute.
70. The method according to any of numbered embodiments 1 to 55, the compound of Formula A (or a pharmaceutically acceptable salt and/or solvate thereof) for use according to any of numbered embodiments 1 to 55, or the use of a compound of Formula A (or a pharmaceutically acceptable salt and/or solvate thereof) according to any of numbered embodiments 1 to 55,
   wherein the compound of Formula A (or a pharmaceutically acceptable salt and/or solvate thereof) is administered as a suspension.
71. The method according to numbered embodiment 70, the compound of Formula A (or a pharmaceutically acceptable salt and/or solvate thereof) for use according to numbered embodiment 70, or the use of a compound of Formula A (or a pharmaceutically acceptable salt and/or solvate thereof) according to numbered embodiment 70,
   wherein the oral dosage form comprises one or more of sorbitol solution (70%), glycerol, dispersible cellulose, sodium carboxymethylcellulose, polysorbate 80, methyl hydroxybenzoate, sodium benzoate, sodium saccharin, ethanol, and vanilla flavour.
72. The method according to any of numbered embodiments 1 to 55, the compound of Formula A (or a pharmaceutically acceptable salt and/or solvate thereof) for use according to any of numbered embodiments 1 to 55, or the use of a compound of Formula A (or a pharmaceutically acceptable salt and/or solvate thereof) according to any of numbered embodiments 1 to 55,
   wherein the compound of Formula A (or a pharmaceutically acceptable salt and/or solvate thereof) is administered as a chewable tablet or soft gel.
73. The method according to numbered embodiment 72, the compound of Formula A (or a pharmaceutically acceptable salt and/or solvate thereof) for use according to numbered embodiment 72, or the use of a compound of Formula A (or a pharmaceutically acceptable salt and/or solvate thereof) according to any of numbered embodiment 72,
   wherein the compound of Formula A (or a pharmaceutically acceptable salt and/or solvate thereof) is administered as a chewable tablet comprising one or more of mannitol, microcrystalline cellulose, hydroxypropyl cellulose, croscarmellose sodium, strawberry flavour, sucralose, and magnesium stearate.
74. The method according to any of numbered embodiments 56 to 73, the compound of Formula A (or a pharmaceutically acceptable salt and/or solvate thereof) for use according to any of numbered embodiments 56 to 73, or the use of a compound of Formula A (or a pharmaceutically acceptable salt and/or solvate thereof) according to any of numbered embodiments 56 to 73,
   wherein the dissolution specification can be ≥80% in less than about 45 minutes.
75. The method according to numbered embodiment 74, the compound of Formula A (or a pharmaceutically acceptable salt and/or solvate thereof) for use according to numbered embodiment 74, or the use of a compound of Formula A (or a pharmaceutically acceptable salt and/or solvate thereof) according to numbered embodiment 74,
   wherein the dissolution specification can be >80% in less than about 5 minutes.
76. The method according to any of numbered embodiments 1 to 55, the compound of Formula A (or a pharmaceutically acceptable salt and/or solvate thereof) for use according to any of numbered embodiments 1 to 55, or the use of a compound of Formula A (or a pharmaceutically acceptable salt and/or solvate thereof) according to any of numbered embodiments 1 to 55,
   wherein the compound of Formula A (or a pharmaceutically acceptable salt and/or solvate thereof) is administered as a solution.
77. The method according to any of numbered embodiments 1 to 55, the compound of Formula A (or a pharmaceutically acceptable salt and/or solvate thereof) for use according to any of numbered embodiments 1 to 55, or the use of a compound of Formula A (or a pharmaceutically acceptable salt and/or solvate thereof) according to any of numbered embodiments 1 to 55,
   wherein the compound of Formula A (or a pharmaceutically acceptable salt and/or solvate thereof) is administered as an emulsion.
78. The method according to any of numbered embodiments 1 to 55, the compound of Formula A (or a pharmaceutically acceptable salt and/or solvate thereof) for use according to any of numbered embodiments 1 to 55, or the use of a compound of Formula A (or a pharmaceutically acceptable salt and/or solvate thereof) according to any of numbered embodiments 1 to 55,
   wherein the compound of Formula A (or a pharmaceutically acceptable salt and/or solvate thereof) is administered as a syrup.

1-42. (canceled)

43. A method for treating bradykinin mediated angioedema on-demand comprising: orally administering the compound of Formula A (or a pharmaceutically acceptable salt and/or solvate thereof) to a patient in need thereof on-demand, wherein the compound of Formula A (or a pharmaceutically acceptable salt and/or solvate thereof) is administered as an oral dosage form that is a chewable tablet or soft gel, a mini-tablet, a powder, granules, a solution, an emulsion, a suspension, a syrup, a dispersible tablet, or an orodispersible tablet, wherein the compound of Formula A is:

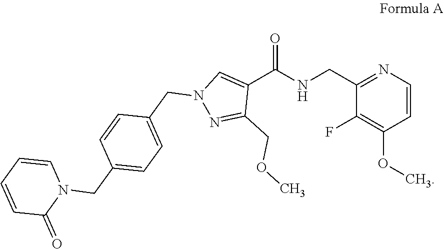 [SMILES: COCc1nn(Cc2ccc(Cn3ccccc3=O)cc2)cc1C(=O)NCc1nccc(OC)c1F]

44. (canceled)

45. (canceled)

46. The method according to claim 43, wherein the bradykinin mediated angioedema is hereditary angioedema (HAE); or wherein the bradykinin mediated angioedema is bradykinin-mediated angioedema non-hereditary (BK-AEnH).

47. The method according to claim 46, wherein the bradykinin mediated angioedema is hereditary angioedema (HAE).

48. The method according to claim 46, wherein the bradykinin mediated angioedema is bradykinin-mediated angioedema non-hereditary (BK-AEnH).

49. The method according to claim 48, wherein the bradykinin-mediated angioedema non-hereditary (BK-AEnH) is not caused by an inherited genetic dysfunction, fault, or mutation.

50. The method according to claim 49, wherein the BK-AEnH is: non-hereditary angioedema with normal C1 Inhibitor (AE-nC1 Inh), optionally environmental, hormonal, or drug-induced; acquired angioedema; anaphylaxis associated angioedema; angiotensin converting enzyme (ACE) inhibitor-induced angioedema; dipeptidyl peptidase-4 inhibitor-induced angioedema; and tPA-induced angioedema (tissue plasminogen activator-induced angioedema).

51. The method according to claim 49, wherein the AE-nC1 Inh is environmentally-induced by air pollution and/or silver nanoparticles.

52. The method according to claim 43, wherein the patient is aged between 2 and less than 18.

53. The method according to claim 52, wherein the patient is aged between 2 and less than 12.

54. The method according to claim 43, wherein the patient is aged 70 years or older.

55. The method according to claim 43, wherein the compound of Formula A (or a pharmaceutically acceptable salt and/or solvate thereof) is for use in treating an attack of bradykinin mediated angioedema on-demand and is orally administered on-demand upon recognition of a symptom of a bradykinin mediated angioedema attack.

56. The method according to claim 55, wherein the patient is suffering from a laryngeal attack.

57. The method according to claim 55, wherein the symptom of an acute HAE attack recognised is at least one of: swelling of tissues; fatigue; headache; muscle aches; skin tingling; abdominal pain; nausea; vomiting; diarrhoea; difficulty swallowing; hoarseness; shortness of breath; and/or mood changes.

58. The method according to claim 55, wherein the compound of Formula A (or a pharmaceutically acceptable salt and/or solvate thereof) is orally administered on-demand within 1 hour of the symptom of a bradykinin mediated angioedema attack being recognised.

59. The method according to claim 55, wherein the compound of Formula A (or a pharmaceutically acceptable salt and/or solvate thereof) is orally administered on-demand within 30 minutes, within 20 minutes, within 10 minutes, or within 5 minutes of the symptom of a bradykinin mediated angioedema attack being recognised.

60. The method according to claim 55, wherein the compound of Formula A (or a pharmaceutically acceptable salt or solvate thereof) is orally administered on-demand in the prodromal phase of a bradykinin mediated angioedema attack.

61. The method according to claim 60, wherein the symptom recognised is at least one of: a slight swelling, abdominal pain or reddening of the skin.

62. The method according to claim 61, wherein the symptom recognised is erythema marginatum.

63. The method according to claim 43, wherein the treatment shortens the duration of the bradykinin mediated angioedema attack.

64. The method according to claim 60, wherein the treatment prevents the bradykinin mediated angioedema attack from progressing to the swelling stage of a bradykinin mediated angioedema attack.

65. The method according to claim 43, wherein the compound of Formula A (or a pharmaceutically acceptable salt and/or solvate thereof) is orally administered on-demand to prophylactically reduce the likelihood of a bradykinin mediated angioedema attack.

66. The method according to claim 65, wherein the compound of Formula A (or a pharmaceutically acceptable salt and/or solvate thereof) is orally administered on-demand when it is anticipated that a bradykinin mediated angioedema attack will be induced.

67. The method according to claim 65, wherein the compound of Formula A (or a pharmaceutically acceptable salt and/or solvate thereof) is orally administered on-demand to prevent a bradykinin mediated angioedema attack.

68. The method according to claim 65, wherein the BK-AEnH is tPA-induced angioedema, wherein the patient is also being administered a tissue plasminogen activator, wherein the compound of Formula A (or a pharmaceutically acceptable salt and/or solvate thereof) is administered prior to, during, or after administration of the tissue plasminogen activator to the patient.

69. The method according to claim 66, wherein the compound of Formula A (or a pharmaceutically acceptable salt and/or solvate thereof) is orally administered on-demand when it is anticipated that a bradykinin mediated angioedema attack will be induced by physical traumata and/or stress.

70. The method according to claim 69, wherein it is anticipated that a bradykinin mediated angioedema attack will be induced by the physical traumata of a dental procedure and/or the mental stress associated with a dental procedure.

71. The method according to claim 43, wherein the compound of Formula A (or a pharmaceutically acceptable salt and/or solvate thereof) is administered as a dispersible tablet or an orodispersible tablet.

72. The method according to claim 71, wherein the compound of Formula A (or a pharmaceutically acceptable salt and/or solvate thereof) is administered as a dispersible tablet.

73. The method according to claim 72, wherein the compound of Formula A (or a pharmaceutically acceptable salt and/or solvate thereof) is administered as an orodispersible tablet.

74. The method according to claim 73, wherein the orodispersible tablet comprises one or more of microcrystalline cellulose, sodium starch glycolate, croscarmellose sodium, camphor, sodium saccharin, and magnesium stearate.

75. The method according to claim 73, wherein the orodispersible tablet comprises polyvinyl polypyrrolidone.

76. The method according to claim 73, wherein the orodispersible tablet comprises one or more of pearlitol Flash (co-processed Mannitol/Maize starch), sucralose, and sodium stearyl fumarate.

77. The method according to claim 43, wherein the compound of Formula A (or a pharmaceutically acceptable salt and/or solvate thereof) is administered as a mini-tablet.

78. The method according to claim 77, wherein the mini-tablet comprises one or more of microcrystalline cellulose, croscarmellose sodium, polyvinylpyrrolidone (povidone), and magnesium stearate.

79. The method according to claim 43, wherein the compound of Formula A (or a pharmaceutically acceptable salt and/or solvate thereof) is administered as a orodispersible film.

80. The method according to claim 71, wherein the disintegration specification is >80% in less than about 10 minutes.

81. The method according to claim 80, wherein the disintegration specification is >80% in less than about 1 minute.

82. The method according to claim 71, wherein the dissolution specification is ≥80% in less than about 45 minutes.

83. The method according to claim 82, wherein the dissolution specification is >80% in less than about 5 minutes.

84. The method according to claim 75, wherein the polyvinyl polypyrrolidone is crospovidone.
